SEQUENCE LISTING (240 sequences)

SEQ ID NO: 1 - DNA, 33 bp, Artificial sequence
  Synthetic oligonucleotide
cataacacatctcacaatccatctcaccaccca

SEQ ID NO: 2 - DNA, 23 bp, Artificial sequence
  Synthetic oligonucleotide
gatggattgtgagatgtgttatg

SEQ ID NO: 3 - DNA, 21 bp, Artificial sequence
  Synthetic oligonucleotide
cataacacatctcacaatcca

SEQ ID NO: 4 - DNA, 24 bp, Artificial sequence
  Synthetic oligonucleotide
caatccatctcaccacccatctca

SEQ ID NO: 5 - DNA, 29 bp, Artificial sequence
  Synthetic oligonucleotide
tgagatgggtggtgagatggattgtgaga

SEQ ID NO: 6 - DNA, 29 bp, Artificial sequence
  Synthetic oligonucleotide
tctcacaatccatctcaccacccatctca

SEQ ID NO: 7 - DNA, 23 bp, Artificial sequence
  Synthetic oligonucleotide
cacaatccatctcaccacccact

SEQ ID NO: 8 - DNA, 30 bp, Artificial sequence
  Synthetic oligonucleotide
agtgggtggtgagatggattgtgagatgtg

SEQ ID NO: 9 - DNA, 33 bp, Artificial sequence
  Synthetic oligonucleotide
caatctacatctcaacactcatctcattcctca

SEQ ID NO: 10 - DNA, 23 bp, Artificial sequence
  Synthetic oligonucleotide
gatgagtgttgagatgtagattg

SEQ ID NO: 11 - DNA, 21 bp, Artificial sequence
  Synthetic oligonucleotide
caatctacatctcaacactca

SEQ ID NO: 12 - DNA, 26 bp, Artificial sequence
  Synthetic oligonucleotide
caacactcatctcattcctcatctca

SEQ ID NO: 13 - DNA, 31 bp, Artificial sequence
  Synthetic oligonucleotide
tgagatgaggaatgagatgagtgttgagatg

SEQ ID NO: 14 - DNA, 31 bp, Artificial sequence
  Synthetic oligonucleotide
catctcaacactcatctcattcctcatctca

SEQ ID NO: 15 - DNA, 20 bp, Artificial sequence
  Synthetic oligonucleotide
ccataacacatctcacaatc

SEQ ID NO: 16 - DNA, 25 bp, Artificial sequence
  Synthetic oligonucleotide
gatggattgtgagatgtgttatggt

SEQ ID NO: 17 - DNA, 38 bp, Artificial sequence
  Synthetic oligonucleotide
cgtattggacatttccgtagaccgactggacatcttcg

SEQ ID NO: 18 - DNA, 24 bp, Artificial sequence
  Synthetic oligonucleotide
tggtccttcacaccaatacggcat

SEQ ID NO: 19 - DNA, 42 bp, Artificial sequence
  Synthetic oligonucleotide
tctacggaaatgtggcagaatcaatcataagacaccagtcgg

SEQ ID NO: 20 - DNA, 26 bp, Artificial sequence
  Synthetic oligonucleotide
cagacgaagatgtggtagtggaatgc

SEQ ID NO: 21 - DNA, 38 bp, Artificial sequence
  Synthetic oligonucleotide
tccactacctgtcttatgattgattctgcctgtgaagg

SEQ ID NO: 22 - DNA, 38 bp, Artificial sequence
  Synthetic oligonucleotide
ctcagtggacagccgttctggagcgttggacgaaactc

SEQ ID NO: 23 - DNA, 26 bp, Artificial sequence
  Synthetic oligonucleotide
tttctggtagagcaccactgagaggt

SEQ ID NO: 24 - DNA, 42 bp, Artificial sequence
  Synthetic oligonucleotide
ccagaacggctgtggctaaacagtaaccgaagcaccaacgct

SEQ ID NO: 25 - DNA, 26 bp, Artificial sequence
  Synthetic oligonucleotide
accagagtttcgtggtcatcgtacct

SEQ ID NO: 26 - DNA, 38 bp, Artificial sequence
  Synthetic oligonucleotide
acgatgacctgcttcggttactgtttagcctgctctac

SEQ ID NO: 27 - DNA, 18 bp, Artificial sequence
  Synthetic oligonucleotide
tattggtgtgaaggacca

SEQ ID NO: 28 - DNA, 18 bp, Artificial sequence
  Synthetic oligonucleotide
cagtggtgctctaccaga

SEQ ID NO: 29 - DNA, 54 bp, Artificial sequence
  Synthetic oligonucleotide
tgagtttcaaaggaacgtccaccgttttcggtggacttaactaaagatctccaa

SEQ ID NO: 30 - DNA, 54 bp, Artificial sequence
  Synthetic oligonucleotide
aaaaaaggcttttgcggtggtccgttttcggaccacttggatcgtcgggtagca

SEQ ID NO: 31 - DNA, 54 bp, Artificial sequence
  Synthetic oligonucleotide
acggctacaagtacaactcggcacttttgtgccgagttcggagattcgcgacct

SEQ ID NO: 32 - DNA, 54 bp, Artificial sequence
  Synthetic oligonucleotide
gctccatgacgtaacacggatcgcttttgcgatccgttaagctgctacaccaga

SEQ ID NO: 33 - DNA, 54 bp, Artificial sequence
  Synthetic oligonucleotide
acgagtagatcagttgcaccgctgttttcagcggtgttagatttagcgccaaaa

SEQ ID NO: 34 - DNA, 54 bp, Artificial sequence
  Synthetic oligonucleotide
ggaattaccaccacccgtgaggcgttttcgcctcactttcattttccgtaacac

SEQ ID NO: 35 - DNA, 54 bp, Artificial sequence
  Synthetic oligonucleotide
gagaataggtcaccagcggaaccgttttcggttccgtttacaaactccgccacc

SEQ ID NO: 36 - DNA, 54 bp, Artificial sequence
  Synthetic oligonucleotide
aaaggccgctccaaaaccgtggcgttttcgccacggttggagccttagcggagt

SEQ ID NO: 37 - DNA, 54 bp, Artificial sequence
  Synthetic oligonucleotide
gcgaaacaagaggcttgtgctgcgttttcgcagcactttgaggactagggagtt

SEQ ID NO: 38 - DNA, 54 bp, Artificial sequence
  Synthetic oligonucleotide
ccaaatcattacttagacgctggcttttgccagcgtttccggaacgtaccaagc

SEQ ID NO: 39 - DNA, 54 bp, Artificial sequence
  Synthetic oligonucleotide
aaagattctaaattggcgacggacttttgtccgtcgttgcttgagattcattac

SEQ ID NO: 40 - DNA, 54 bp, Artificial sequence
  Synthetic oligonucleotide
ctcagagcgaggcataggctccgcttttgcggagccttgtaagagcacaggtag

SEQ ID NO: 41 - DNA, 54 bp, Artificial sequence
  Synthetic oligonucleotide
tgtagcataactttcaggcatccgttttcggatgccttacagtttctaattgta

SEQ ID NO: 42 - DNA, 54 bp, Artificial sequence
  Synthetic oligonucleotide
tcggtttaggtcgctggctgacgcttttgcgtcagcttaggcttgcaaagactt

SEQ ID NO: 43 - DNA, 54 bp, Artificial sequence
  Synthetic oligonucleotide
tttcatgatgacccccaccagccgttttcggctggtttagcgattaaggcgcag

SEQ ID NO: 44 - DNA, 54 bp, Artificial sequence
  Synthetic oligonucleotide
acggtcaatgacaagacggaggcgttttcgcctccgttaccggatatggtttaa

SEQ ID NO: 45 - DNA, 54 bp, Artificial sequence
  Synthetic oligonucleotide
tttcaactacggaacactcgctgcttttgcagcgagttacattattaacactat

SEQ ID NO: 46 - DNA, 54 bp, Artificial sequence
  Synthetic oligonucleotide
cataacccaccgccacctggctcgttttcgagccagttcctcagaaacaacgcc

SEQ ID NO: 47 - DNA, 54 bp, Artificial sequence
  Synthetic oligonucleotide
tgctaaactccacagagccagtgcttttgcactggcttcagccctctaccgcca

SEQ ID NO: 48 - DNA, 54 bp, Artificial sequence
  Synthetic oligonucleotide
atatattctcagcttgccgtccgcttttgcggacggttctttcgagtgggattt

SEQ ID NO: 49 - DNA, 54 bp, Artificial sequence
  Synthetic oligonucleotide
ctcatcttggaagtttcggatggcttttgccatccgttccattaaacataaccg

SEQ ID NO: 50 - DNA, 54 bp, Artificial sequence
  Synthetic oligonucleotide
agtaatcttcataaggtctggtcgttttcgaccagattgaaccgaactaaaaca

SEQ ID NO: 51 - DNA, 54 bp, Artificial sequence
  Synthetic oligonucleotide
acgaactattaatcatggcacctgttttcaggtgcctttgtgaatttcatcaag

SEQ ID NO: 52 - DNA, 54 bp, Artificial sequence
  Synthetic oligonucleotide
ccctcagatcgtttaccgcttgcgttttcgcaagcgttcagacgacttaataaa

SEQ ID NO: 53 - DNA, 54 bp, Artificial sequence
  Synthetic oligonucleotide
cgtaacgaaaatgaatcctgcctgttttcaggcaggtttttctgtagtgaattt

SEQ ID NO: 54 - DNA, 54 bp, Artificial sequence
  Synthetic oligonucleotide
cttaaacaacaaccatcggtgccgttttcggcaccgttcgcccacgcgggtaaa

SEQ ID NO: 55 - DNA, 54 bp, Artificial sequence
  Synthetic oligonucleotide
atacgtaagaggcaaactcggtcgttttcgaccgagttagaatacactgaccaa

SEQ ID NO: 56 - DNA, 54 bp, Artificial sequence
  Synthetic oligonucleotide
ctttgaaaataggctgccgaggacttttgtcctcggttgctgacctaccttatg

SEQ ID NO: 57 - DNA, 54 bp, Artificial sequence
  Synthetic oligonucleotide
cgattttaggaagaaacggcaggcttttgcctgccgttaatctacggataaaaa

SEQ ID NO: 58 - DNA, 54 bp, Artificial sequence
  Synthetic oligonucleotide
ccaaaatatactcaggtgcggtcgttttcgaccgcattaggtttagatagttag

SEQ ID NO: 59 - DNA, 54 bp, Artificial sequence
  Synthetic oligonucleotide
acgttagttctaaagtcgcttggcttttgccaagcgtttttgtcgtgatacagg

SEQ ID NO: 60 - DNA, 54 bp, Artificial sequence
  Synthetic oligonucleotide
caatgacagcttgatatggcgagcttttgctcgccattccgatagtctccctca

SEQ ID NO: 61 - DNA, 54 bp, Artificial sequence
  Synthetic oligonucleotide
aaacgaaatgccactaccacctcgttttcgaggtggttcgaaggcagccagcaa

SEQ ID NO: 62 - DNA, 54 bp, Artificial sequence
  Synthetic oligonucleotide
ccaggcgcgaggacagctctggacttttgtccagagttatgaacgggtagaaaa

SEQ ID NO: 63 - DNA, 54 bp, Artificial sequence
  Synthetic oligonucleotide
ggacgttgagaactggcgaggcacttttgtgcctcgttctcattatgcgctaat

SEQ ID NO: 64 - DNA, 54 bp, Artificial sequence
  Synthetic oligonucleotide
tatcaccggcgagaggctgcgtcgttttcgacgcagttcttttgcaatcctgaa

SEQ ID NO: 65 - DNA, 54 bp, Artificial sequence
  Synthetic oligonucleotide
agtgtactatacatggctcctgcgttttcgcaggagttcttttgatctttccag

SEQ ID NO: 66 - DNA, 54 bp, Artificial sequence
  Synthetic oligonucleotide
gagccgccccaccaccgtcaggcgttttcgcctgacttggaaccgctgcgccga

SEQ ID NO: 67 - DNA, 54 bp, Artificial sequence
  Synthetic oligonucleotide
aatcaccaccatttggcgtcctgcttttgcaggacgttgaattagaccaaccta

SEQ ID NO: 68 - DNA, 54 bp, Artificial sequence
  Synthetic oligonucleotide
tacatacacagtatgtcggacctgttttcaggtccgtttagcaaactgtacaga

SEQ ID NO: 69 - DNA, 54 bp, Artificial sequence
  Synthetic oligonucleotide
atcagagagtcagaggcgaggtcgttttcgacctcgttgtaattgaaccagtca

SEQ ID NO: 70 - DNA, 54 bp, Artificial sequence
  Synthetic oligonucleotide
tcttaccatataagtaccgaggcgttttcgcctcggtttagcccggaataggtg

SEQ ID NO: 71 - DNA, 54 bp, Artificial sequence
  Synthetic oligonucleotide
taagcgtcggtaataacaggagcgttttcgctcctgttgttttaacccgtcgag

SEQ ID NO: 72 - DNA, 54 bp, Artificial sequence
  Synthetic oligonucleotide
aaccagagaccctcaggcagtcgcttttgcgactgcttaaccgccacgttccag

SEQ ID NO: 73 - DNA, 54 bp, Artificial sequence
  Synthetic oligonucleotide
gacttgaggtagcaccgtctggcgttttcgccagacttattaccatatcaccgg

SEQ ID NO: 74 - DNA, 54 bp, Artificial sequence
  Synthetic oligonucleotide
ttattacgtaaaggtgtggctgcgttttcgcagccattgcaacataccgtcacc

SEQ ID NO: 75 - DNA, 54 bp, Artificial sequence
  Synthetic oligonucleotide
tgaacaaagataacccagtgcctgttttcaggcactttacaagaataagactcc

SEQ ID NO: 76 - DNA, 54 bp, Artificial sequence
  Synthetic oligonucleotide
agggttgaacgctaacgccaggacttttgtcctggcttgagcgtctgaacaccc

SEQ ID NO: 77 - DNA, 54 bp, Artificial sequence
  Synthetic oligonucleotide
tgccttgacagtctctgtcggtgcttttgcaccgacttgaatttacccctcaga

SEQ ID NO: 78 - DNA, 54 bp, Artificial sequence
  Synthetic oligonucleotide
gccaccactcttttcacggtcggcttttgccgaccgtttaatcaaatagcaagg

SEQ ID NO: 79 - DNA, 54 bp, Artificial sequence
  Synthetic oligonucleotide
ccggaaactaaaggtggacctggcttttgccaggtcttaattatcataaaagaa

SEQ ID NO: 80 - DNA, 54 bp, Artificial sequence
  Synthetic oligonucleotide
acgcaaagaagaactgtcggctcgttttcgagccgattgcatgatttgagttaa

SEQ ID NO: 81 - DNA, 54 bp, Artificial sequence
  Synthetic oligonucleotide
gcccaatagacgggagcacaggcgttttcgcctgtgttaattaactttccagag

SEQ ID NO: 82 - DNA, 54 bp, Artificial sequence
  Synthetic oligonucleotide
cctaatttaccaggcgtcggagcgttttcgctccgattgataagtgggggtcag

SEQ ID NO: 83 - DNA, 54 bp, Artificial sequence
  Synthetic oligonucleotide
ggaaagcggtaacagtgtggcagcttttgctgccacttgcccgtatcggggttt

SEQ ID NO: 84 - DNA, 54 bp, Artificial sequence
  Synthetic oligonucleotide
gtttgccacctcagagaccaggcgttttcgcctggtttccgccaccgccagaat

SEQ ID NO: 85 - DNA, 54 bp, Artificial sequence
  Synthetic oligonucleotide
ttattcatgtcaccaagctcgctgttttcagcgagctttgaaaccattattagc

SEQ ID NO: 86 - DNA, 54 bp, Artificial sequence
  Synthetic oligonucleotide
atacccaaacaccacgcctaccgcttttgcggtaggttgaataagtgacggaaa

SEQ ID NO: 87 - DNA, 54 bp, Artificial sequence
  Synthetic oligonucleotide
gcgcattaataagagcctggacgcttttgcgtccagttaagaaacaataacgga

SEQ ID NO: 88 - DNA, 54 bp, Artificial sequence
  Synthetic oligonucleotide
tgctcagtgccagttaggtggtcgttttcgaccaccttcaaaataaacagggaa

SEQ ID NO: 89 - DNA, 54 bp, Artificial sequence
  Synthetic oligonucleotide
aatgccccataaatccgctcggacttttgtccgagctttcattaaaagaaccac

SEQ ID NO: 90 - DNA, 54 bp, Artificial sequence
  Synthetic oligonucleotide
caccagagttcggtcagccgagcgttttcgctcggctttagccccctcgatagc

SEQ ID NO: 91 - DNA, 54 bp, Artificial sequence
  Synthetic oligonucleotide
agcaccgtagggaaggtcggaggcttttgcctccgatttaaatattttattttg

SEQ ID NO: 92 - DNA, 54 bp, Artificial sequence
  Synthetic oligonucleotide
tcacaatcccgaggaactggtggcttttgccaccagttacgcaataatgaaata

SEQ ID NO: 93 - DNA, 54 bp, Artificial sequence
  Synthetic oligonucleotide
gcaatagcagagaataccgcaggcttttgcctgcggttacataaaaacagccat

SEQ ID NO: 94 - DNA, 54 bp, Artificial sequence
  Synthetic oligonucleotide
attatttagaaggattgccatcgcttttgcgatggcttaggattagaaacagtt

SEQ ID NO: 95 - DNA, 54 bp, Artificial sequence
  Synthetic oligonucleotide
acaaacaactgcctatcacgacgcttttgcgtcgtgttttcggaacctgagact

SEQ ID NO: 96 - DNA, 54 bp, Artificial sequence
  Synthetic oligonucleotide
tcggcattccgccgccgtcgctgcttttgcagcgacttagcattgatgatattc

SEQ ID NO: 97 - DNA, 54 bp, Artificial sequence
  Synthetic oligonucleotide
attgagggaatcagtacggagcacttttgtgctccgttgcgacagacgttttca

SEQ ID NO: 98 - DNA, 54 bp, Artificial sequence
  Synthetic oligonucleotide
gaaggaaaaatagaaagcctagcgttttcgctaggcttattcatatttcaaccg

SEQ ID NO: 99 - DNA, 54 bp, Artificial sequence
  Synthetic oligonucleotide
ctttacagtatcttaccgctcgtgttttcacgagcgttcgaagcccagttacca

SEQ ID NO: 100 - DNA, 54 bp, Artificial sequence
  Synthetic oligonucleotide
cctcaagatcccaatccgtggagcttttgctccacgttcaaataagatagcagc

SEQ ID NO: 101 - DNA, 46 bp, Artificial sequence
  Synthetic oligonucleotide
agggatagcaagcccacaacgtgaggacacttggaggctgcactcg

SEQ ID NO: 102 - DNA, 42 bp, Artificial sequence
  Synthetic oligonucleotide
tgtcctcacgttgctggatgccgatcctacgacacctccaag

SEQ ID NO: 103 - DNA, 46 bp, Artificial sequence
  Synthetic oligonucleotide
tcgctgacttgtcgtaggatcggcatccagataggaacccatgtac

SEQ ID NO: 104 - DNA, 26 bp, Artificial sequence
  Synthetic oligonucleotide
cagacgagtgcagagtcagcgaatgc

SEQ ID NO: 105 - DNA, 46 bp, Artificial sequence
  Synthetic oligonucleotide
gaattgcgaataataagtgaccttgctgtaccgtcgagatggagtc

SEQ ID NO: 106 - DNA, 42 bp, Artificial sequence
  Synthetic oligonucleotide
acagcaaggtcaccgcagttggcactaggcgacatcgacggt

SEQ ID NO: 107 - DNA, 46 bp, Artificial sequence
  Synthetic oligonucleotide
accacaacctgtcgcctagtgccaactgcgttttttcacgttgaaa

SEQ ID NO: 108 - DNA, 26 bp, Artificial sequence
  Synthetic oligonucleotide
accagactccatcggttgtggtacct

SEQ ID NO: 109 - DNA, 46 bp, Artificial sequence
  Synthetic oligonucleotide
accctcagcagcgaaacgagtacggcaacacggtgagagcctacgg

SEQ ID NO: 110 - DNA, 42 bp, Artificial sequence
  Synthetic oligonucleotide
gttgccgtactcgactggtcacgaacgtctccaactcaccgt

SEQ ID NO: 111 - DNA, 46 bp, Artificial sequence
  Synthetic oligonucleotide
tgctctgccttggagacgttcgtgaccagtgacagcatcggaacga

SEQ ID NO: 112 - DNA, 26 bp, Artificial sequence
  Synthetic oligonucleotide
cagaccgtaggctggcagagcaatgc

SEQ ID NO: 113 - DNA, 46 bp, Artificial sequence
  Synthetic oligonucleotide
tgtatcatcgcctgatcaacggtacgagatgcgaagcacagagtgc

SEQ ID NO: 114 - DNA, 42 bp, Artificial sequence
  Synthetic oligonucleotide
tctcgtaccgttgccagtagacctagccgacgtggcttcgca

SEQ ID NO: 115 - DNA, 46 bp, Artificial sequence
  Synthetic oligonucleotide
agtcacgctcacgtcggctaggtctactggaaattgtgtcgaaatc

SEQ ID NO: 116 - DNA, 26 bp, Artificial sequence
  Synthetic oligonucleotide
accagcactctgtagcgtgactacct

SEQ ID NO: 117 - DNA, 46 bp, Artificial sequence
  Synthetic oligonucleotide
cattcagtgaataaggacgctatgcctatcgctctaggacctctgg

SEQ ID NO: 118 - DNA, 42 bp, Artificial sequence
  Synthetic oligonucleotide
ataggcatagcgttgctccagtctgctgctcaggctagagcg

SEQ ID NO: 119 - DNA, 46 bp, Artificial sequence
  Synthetic oligonucleotide
tccacgactcctgagcagcagactggagcacttgccctgacgagaa

SEQ ID NO: 120 - DNA, 26 bp, Artificial sequence
  Synthetic oligonucleotide
cagaccagaggtcagtcgtggaatgc

SEQ ID NO: 121 - DNA, 46 bp, Artificial sequence
  Synthetic oligonucleotide
gaataccacattcaacaccgatgaggatcacggcactcgacactgc

SEQ ID NO: 122 - DNA, 42 bp, Artificial sequence
  Synthetic oligonucleotide
gatcctcatcggtcaagcgaaggtgcgagcctgtagtgccgt

SEQ ID NO: 123 - DNA, 46 bp, Artificial sequence
  Synthetic oligonucleotide
agcggactgacaggctcgcaccttcgcttgtaatgcagatacataa

SEQ ID NO: 124 - DNA, 26 bp, Artificial sequence
  Synthetic oligonucleotide
accagcagtgtcgcagtccgctacct

SEQ ID NO: 125 - DNA, 17 bp, Artificial sequence
  Synthetic oligonucleotide
aagcgtagtcggatctc

SEQ ID NO: 126 - DNA, 17 bp, Artificial sequence
  Synthetic oligonucleotide
aagcgtagtcggatctc

SEQ ID NO: 127 - DNA, 44 bp, Artificial sequence
  Synthetic oligonucleotide
aaattcttaccagtataaagccaacttttgagatccgactacgc

SEQ ID NO: 128 - DNA, 44 bp, Artificial sequence
  Synthetic oligonucleotide
gcctgtttagtatcatatgcgttatttttgagatccgactacgc

SEQ ID NO: 129 - DNA, 44 bp, Artificial sequence
  Synthetic oligonucleotide
acaccggaatcataattactagaaattttgagatccgactacgc

SEQ ID NO: 130 - DNA, 44 bp, Artificial sequence
  Synthetic oligonucleotide
gataaataaggcgttaaataagaatttttgagatccgactacgc

SEQ ID NO: 131 - DNA, 44 bp, Artificial sequence
  Synthetic oligonucleotide
tttaatggtttgaaataccgaccgtttttgagatccgactacgc

SEQ ID NO: 132 - DNA, 44 bp, Artificial sequence
  Synthetic oligonucleotide
ttagttaatttcatcttctgacctattttgagatccgactacgc

SEQ ID NO: 133 - DNA, 44 bp, Artificial sequence
  Synthetic oligonucleotide
acgcgagaaaactttttcaaatatattttgagatccgactacgc

SEQ ID NO: 134 - DNA, 44 bp, Artificial sequence
  Synthetic oligonucleotide
gatgcaaatccaatcgcaagacaaattttgagatccgactacgc

SEQ ID NO: 135 - DNA, 44 bp, Artificial sequence
  Synthetic oligonucleotide
tgggttatataactatatgtaaatgttttgagatccgactacgc

SEQ ID NO: 136 - DNA, 44 bp, Artificial sequence
  Synthetic oligonucleotide
actacctttttaacctccggcttagttttgagatccgactacgc

SEQ ID NO: 137 - DNA, 44 bp, Artificial sequence
  Synthetic oligonucleotide
aatttatcaaaatcataggtctgagttttgagatccgactacgc

SEQ ID NO: 138 - DNA, 44 bp, Artificial sequence
  Synthetic oligonucleotide
ttaagacgctgagaagagtcaatagttttgagatccgactacgc

SEQ ID NO: 139 - DNA, 44 bp, Artificial sequence
  Synthetic oligonucleotide
tccttgaaaacatagcgatagcttattttgagatccgactacgc

SEQ ID NO: 140 - DNA, 44 bp, Artificial sequence
  Synthetic oligonucleotide
tcgctattaattaattttcccttagttttgagatccgactacgc

SEQ ID NO: 141 - DNA, 44 bp, Artificial sequence
  Synthetic oligonucleotide
agtgaataaccttgcttctgtaaatttttgagatccgactacgc

SEQ ID NO: 142 - DNA, 44 bp, Artificial sequence
  Synthetic oligonucleotide
gaaacagtacataaatcaatatatgttttgagatccgactacgc

SEQ ID NO: 143 - DNA, 44 bp, Artificial sequence
  Synthetic oligonucleotide
atttcatttgaattaccttttttaattttgagatccgactacgc

SEQ ID NO: 144 - DNA, 44 bp, Artificial sequence
  Synthetic oligonucleotide
agaaaacaaaattaattacatttaattttgagatccgactacgc

SEQ ID NO: 145 - DNA, 44 bp, Artificial sequence
  Synthetic oligonucleotide
caaaagaagatgatgaaacaaacatttttgagatccgactacgc

SEQ ID NO: 146 - DNA, 44 bp, Artificial sequence
  Synthetic oligonucleotide
gcgaattattcatttcaattacctgttttgagatccgactacgc

SEQ ID NO: 147 - DNA, 44 bp, Artificial sequence
  Synthetic oligonucleotide
aataccaagttacaaaatcgcgcagttttgagatccgactacgc

SEQ ID NO: 148 - DNA, 44 bp, Artificial sequence
  Synthetic oligonucleotide
caataacggattcgcctgattgcttttttgagatccgactacgc

SEQ ID NO: 149 - DNA, 44 bp, Artificial sequence
  Synthetic oligonucleotide
taacagtaccttttacatcgggagattttgagatccgactacgc

SEQ ID NO: 150 - DNA, 44 bp, Artificial sequence
  Synthetic oligonucleotide
caggtttaacgtcagatgaatatacttttgagatccgactacgc

SEQ ID NO: 151 - DNA, 44 bp, Artificial sequence
  Synthetic oligonucleotide
cagaaataaagaaattgcgtagattttttgagatccgactacgc

SEQ ID NO: 152 - DNA, 44 bp, Artificial sequence
  Synthetic oligonucleotide
ccatatcaaaattatttgcacgtaattttgagatccgactacgc

SEQ ID NO: 153 - DNA, 44 bp, Artificial sequence
  Synthetic oligonucleotide
tctgaataatggaagggttagaaccttttgagatccgactacgc

SEQ ID NO: 154 - DNA, 44 bp, Artificial sequence
  Synthetic oligonucleotide
tataatcctgattgtttggattatattttgagatccgactacgc

SEQ ID NO: 155 - DNA, 44 bp, Artificial sequence
  Synthetic oligonucleotide
gattatcagatgatggcaattcatcttttgagatccgactacgc

SEQ ID NO: 156 - DNA, 44 bp, Artificial sequence
  Synthetic oligonucleotide
aaggagcggaattatcatcatattcttttgagatccgactacgc

SEQ ID NO: 157 - DNA, 44 bp, Artificial sequence
  Synthetic oligonucleotide
cattttgcggaacaaagaaaccaccttttgagatccgactacgc

SEQ ID NO: 158 - DNA, 44 bp, Artificial sequence
  Synthetic oligonucleotide
taattttaaaagtttgagtaacattttttgagatccgactacgc

SEQ ID NO: 159 - DNA, 44 bp, Artificial sequence
  Synthetic oligonucleotide
gtattaaatcctttgcccgaacgttttttgagatccgactacgc

SEQ ID NO: 160 - DNA, 44 bp, Artificial sequence
  Synthetic oligonucleotide
tagactttacaaacaattcgacaacttttgagatccgactacgc

SEQ ID NO: 161 - DNA, 44 bp, Artificial sequence
  Synthetic oligonucleotide
ataatacatttgaggatttagaagtttttgagatccgactacgc

SEQ ID NO: 162 - DNA, 44 bp, Artificial sequence
  Synthetic oligonucleotide
caactaatagattagagccgtcaatttttgagatccgactacgc

SEQ ID NO: 163 - DNA, 44 bp, Artificial sequence
  Synthetic oligonucleotide
tatctaaaatatctttaggagcactttttgagatccgactacgc

SEQ ID NO: 164 - DNA, 44 bp, Artificial sequence
  Synthetic oligonucleotide
actgatagccctaaaacatcgccatttttgagatccgactacgc

SEQ ID NO: 165 - DNA, 44 bp, Artificial sequence
  Synthetic oligonucleotide
gaatggctattagtctttaatgcgcttttgagatccgactacgc

SEQ ID NO: 166 - DNA, 44 bp, Artificial sequence
  Synthetic oligonucleotide
agaatacgtggcacagacaatatttttttgagatccgactacgc

SEQ ID NO: 167 - DNA, 44 bp, Artificial sequence
  Synthetic oligonucleotide
atagaacccttctgacctgaaagcgttttgagatccgactacgc

SEQ ID NO: 168 - DNA, 44 bp, Artificial sequence
  Synthetic oligonucleotide
ataaaagggacattctggccaacagttttgagatccgactacgc

SEQ ID NO: 169 - DNA, 44 bp, Artificial sequence
  Synthetic oligonucleotide
gcagattcaccagtcacacgaccagttttgagatccgactacgc

SEQ ID NO: 170 - DNA, 44 bp, Artificial sequence
  Synthetic oligonucleotide
atcgtctgaaatggattatttacatttttgagatccgactacgc

SEQ ID NO: 171 - DNA, 44 bp, Artificial sequence
  Synthetic oligonucleotide
atggaaatacctacattttgacgctttttgagatccgactacgc

SEQ ID NO: 172 - DNA, 44 bp, Artificial sequence
  Synthetic oligonucleotide
ccagccattgcaacaggaaaaacgcttttgagatccgactacgc

SEQ ID NO: 173 - DNA, 44 bp, Artificial sequence
  Synthetic oligonucleotide
ctggtaatatccagaacaatattacttttgagatccgactacgc

SEQ ID NO: 174 - DNA, 44 bp, Artificial sequence
  Synthetic oligonucleotide
gtagaagaactcaaactatcggcctttttgagatccgactacgc

SEQ ID NO: 175 - DNA, 44 bp, Artificial sequence
  Synthetic oligonucleotide
tgattagtaataacatcacttgcctttttgagatccgactacgc

SEQ ID NO: 176 - DNA, 44 bp, Artificial sequence
  Synthetic oligonucleotide
aaattaaccgttgtagcaatacttcttttgagatccgactacgc

SEQ ID NO: 177 - DNA, 44 bp, Artificial sequence
  Synthetic oligonucleotide
ccgagtaaaagagtctgtccatcacttttgagatccgactacgc

SEQ ID NO: 178 - DNA, 44 bp, Artificial sequence
  Synthetic oligonucleotide
gaagtgtttttataatcagtgaggcttttgagatccgactacgc

SEQ ID NO: 179 - DNA, 44 bp, Artificial sequence
  Synthetic oligonucleotide
gacaggaacggtacgccagaatcctttttgagatccgactacgc

SEQ ID NO: 180 - DNA, 44 bp, Artificial sequence
  Synthetic oligonucleotide
aacaggaggccgattaaagggatttttttgagatccgactacgc

SEQ ID NO: 181 - DNA, 44 bp, Artificial sequence
  Synthetic oligonucleotide
tcctcgttagaatcagagcgggagcttttgagatccgactacgc

SEQ ID NO: 182 - DNA, 44 bp, Artificial sequence
  Synthetic oligonucleotide
gctttgacgagcacgtataacgtgcttttgagatccgactacgc

SEQ ID NO: 183 - DNA, 44 bp, Artificial sequence
  Synthetic oligonucleotide
cgccgctacagggcgcgtactatggttttgagatccgactacgc

SEQ ID NO: 184 - DNA, 44 bp, Artificial sequence
  Synthetic oligonucleotide
taaccaccacacccgccgcgcttaattttgagatccgactacgc

SEQ ID NO: 185 - DNA, 44 bp, Artificial sequence
  Synthetic oligonucleotide
tggcaagtgtagcggtcacgctgcgttttgagatccgactacgc

SEQ ID NO: 186 - DNA, 44 bp, Artificial sequence
  Synthetic oligonucleotide
aagcgaaaggagcgggcgctagggcttttgagatccgactacgc

SEQ ID NO: 187 - DNA, 44 bp, Artificial sequence
  Synthetic oligonucleotide
cgaacgtggcgagaaaggaagggaattttgagatccgactacgc

SEQ ID NO: 188 - DNA, 44 bp, Artificial sequence
  Synthetic oligonucleotide
gatttagagcttgacggggaaagccttttgagatccgactacgc

SEQ ID NO: 189 - DNA, 44 bp, Artificial sequence
  Synthetic oligonucleotide
taaatcggaaccctaaagggagcccttttgagatccgactacgc

SEQ ID NO: 190 - DNA, 44 bp, Artificial sequence
  Synthetic oligonucleotide
ttttggggtcgaggtgccgtaaagcttttgagatccgactacgc

SEQ ID NO: 191 - DNA, 44 bp, Artificial sequence
  Synthetic oligonucleotide
tacgtgaaccatcacccaaatcaagttttgagatccgactacgc

SEQ ID NO: 192 - DNA, 44 bp, Artificial sequence
  Synthetic oligonucleotide
aaaccgtctatcagggcgatggcccttttgagatccgactacgc

SEQ ID NO: 193 - DNA, 44 bp, Artificial sequence
  Synthetic oligonucleotide
acgtggactccaacgtcaaagggcgttttgagatccgactacgc

SEQ ID NO: 194 - DNA, 44 bp, Artificial sequence
  Synthetic oligonucleotide
tttggaacaagagtccactattaaattttgagatccgactacgc

SEQ ID NO: 195 - DNA, 44 bp, Artificial sequence
  Synthetic oligonucleotide
ccgagatagggttgagtgttgttccttttgagatccgactacgc

SEQ ID NO: 196 - DNA, 44 bp, Artificial sequence
  Synthetic oligonucleotide
aaatcccttataaatcaaaagaatattttgagatccgactacgc

SEQ ID NO: 197 - DNA, 44 bp, Artificial sequence
  Synthetic oligonucleotide
tgtttgatggtggttccgaaatcggttttgagatccgactacgc

SEQ ID NO: 198 - DNA, 44 bp, Artificial sequence
  Synthetic oligonucleotide
ctggtttgccccagcaggcgaaaatttttgagatccgactacgc

SEQ ID NO: 199 - DNA, 44 bp, Artificial sequence
  Synthetic oligonucleotide
tgagagagttgcagcaagcggtccattttgagatccgactacgc

SEQ ID NO: 200 - DNA, 44 bp, Artificial sequence
  Synthetic oligonucleotide
agctgattgcccttcaccgcctggcttttgagatccgactacgc

SEQ ID NO: 201 - DNA, 44 bp, Artificial sequence
  Synthetic oligonucleotide
tttcttttcaccagtgagacgggcattttgagatccgactacgc

SEQ ID NO: 202 - DNA, 44 bp, Artificial sequence
  Synthetic oligonucleotide
gtttgcgtattgggcgccagggtggttttgagatccgactacgc

SEQ ID NO: 203 - DNA, 44 bp, Artificial sequence
  Synthetic oligonucleotide
gaatcggccaacgcgcggggagaggttttgagatccgactacgc

SEQ ID NO: 204 - DNA, 44 bp, Artificial sequence
  Synthetic oligonucleotide
gaaacctgtcgtgccagctgcattattttgagatccgactacgc

SEQ ID NO: 205 - DNA, 44 bp, Artificial sequence
  Synthetic oligonucleotide
tgcgctcactgcccgctttccagtcttttgagatccgactacgc

SEQ ID NO: 206 - DNA, 44 bp, Artificial sequence
  Synthetic oligonucleotide
gagtgagctaactcacattaattgcttttgagatccgactacgc

SEQ ID NO: 207 - DNA, 44 bp, Artificial sequence
  Synthetic oligonucleotide
taaagtgtaaagcctggggtgcctattttgagatccgactacgc

SEQ ID NO: 208 - DNA, 44 bp, Artificial sequence
  Synthetic oligonucleotide
ttccacacaacatacgagccggaagttttgagatccgactacgc

SEQ ID NO: 209 - DNA, 44 bp, Artificial sequence
  Synthetic oligonucleotide
ctgtgtgaaattgttatccgctcacttttgagatccgactacgc

SEQ ID NO: 210 - DNA, 44 bp, Artificial sequence
  Synthetic oligonucleotide
attcgtaatcatggtcatagctgttttttgagatccgactacgc

SEQ ID NO: 211 - DNA, 44 bp, Artificial sequence
  Synthetic oligonucleotide
tagaggatccccgggtaccgagctcttttgagatccgactacgc

SEQ ID NO: 212 - DNA, 44 bp, Artificial sequence
  Synthetic oligonucleotide
caagcttgcatgcctgcaggtcgacttttgagatccgactacgc

SEQ ID NO: 213 - DNA, 44 bp, Artificial sequence
  Synthetic oligonucleotide
acgacgttgtaaaacgacggccagtttttgagatccgactacgc

SEQ ID NO: 214 - DNA, 44 bp, Artificial sequence
  Synthetic oligonucleotide
ttgggtaacgccagggttttcccagttttgagatccgactacgc

SEQ ID NO: 215 - DNA, 44 bp, Artificial sequence
  Synthetic oligonucleotide
agggggatgtgctgcaaggcgattattttgagatccgactacgc

SEQ ID NO: 216 - DNA, 44 bp, Artificial sequence
  Synthetic oligonucleotide
ctcttcgctattacgccagctggcgttttgagatccgactacgc

SEQ ID NO: 217 - DNA, 44 bp, Artificial sequence
  Synthetic oligonucleotide
ctgttgggaagggcgatcggtgcggttttgagatccgactacgc

SEQ ID NO: 218 - DNA, 44 bp, Artificial sequence
  Synthetic oligonucleotide
gcgccattcgccattcaggctgcgcttttgagatccgactacgc

SEQ ID NO: 219 - DNA, 44 bp, Artificial sequence
  Synthetic oligonucleotide
cgcttctggtgccggaaaccaggcattttgagatccgactacgc

SEQ ID NO: 220 - DNA, 44 bp, Artificial sequence
  Synthetic oligonucleotide
atcgcactccagccagctttccggcttttgagatccgactacgc

SEQ ID NO: 221 - DNA, 44 bp, Artificial sequence
  Synthetic oligonucleotide
gacgacgacagtatcggcctcaggattttgagatccgactacgc

SEQ ID NO: 222 - DNA, 44 bp, Artificial sequence
  Synthetic oligonucleotide
gtaaccgtgcatctgccagtttgagttttgagatccgactacgc

SEQ ID NO: 223 - DNA, 44 bp, Artificial sequence
  Synthetic oligonucleotide
ggtcacgttggtgtagatgggcgcattttgagatccgactacgc

SEQ ID NO: 224 - DNA, 44 bp, Artificial sequence
  Synthetic oligonucleotide
aaacggcggattgaccgtaatgggattttgagatccgactacgc

SEQ ID NO: 225 - DNA, 44 bp, Artificial sequence
  Synthetic oligonucleotide
acaacccgtcggattctccgtgggattttgagatccgactacgc

SEQ ID NO: 226 - DNA, 44 bp, Artificial sequence
  Synthetic oligonucleotide
ttcatcaacattaaatgtgagcgagttttgagatccgactacgc

SEQ ID NO: 227 - DNA, 21 bp, Artificial sequence
  Synthetic oligonucleotide
cacaatccatctcaccaccca

SEQ ID NO: 228 - DNA, 28 bp, Artificial sequence
  Synthetic oligonucleotide
tgggtggtgagatggattgtgagatgtg

SEQ ID NO: 229 - DNA, 33 bp, Artificial sequence
  Synthetic oligonucleotide
cacaatccatctcaccacccatctcaaaactca

SEQ ID NO: 230 - DNA, 25 bp, Artificial sequence
  Synthetic oligonucleotide
tgggtggtgagatggattgtgagat

SEQ ID NO: 231 - DNA, 21 bp, Artificial sequence
  Synthetic oligonucleotide
caccacccatctcaaaactca

SEQ ID NO: 232 - DNA, 28 bp, Artificial sequence
  Synthetic oligonucleotide
tgagttttgagatgggtggtgagatgga

SEQ ID NO: 233 - DNA, 33 bp, Artificial sequence
  Synthetic oligonucleotide
caccacccatctcaaaactcatctcatccaaca

SEQ ID NO: 234 - DNA, 24 bp, Artificial sequence
  Synthetic oligonucleotide
tgagttttagatgggtggtgagat

SEQ ID NO: 235 - DNA, 21 bp, Artificial sequence
  Synthetic oligonucleotide
caaaactcatctcatccaaca

SEQ ID NO: 236 - DNA, 28 bp, Artificial sequence
  Synthetic oligonucleotide
tgttggatgagatgagttttgagatggg

SEQ ID NO: 237 - DNA, 21 bp, Artificial sequence
  Synthetic oligonucleotide
tggattgtgagatgtgttatg

SEQ ID NO: 238 - DNA, 16 bp, Artificial sequence
  Synthetic oligonucleotide
cctaccttcacaacta

SEQ ID NO: 239 - DNA, 15 bp, Artificial sequence
  Synthetic oligonucleotide
cttcacaactaatct

SEQ ID NO: 240 - DNA, 41 bp, Artificial sequence
  Synthetic oligonucleotide
tgggtggtgagatggattgtgagattagttgtgaaggtagg

(19) United States
(12) Patent Application Publication
(10) Pub. No.: US 2021/0254070 A1
(43) Pub. Date: Aug. 19, 2021

(54) SYSTEMS AND METHODS RELATED TO OLIGONUCLEOTIDE BUFFERS

(71) Applicant: THE JOHNS HOPKINS UNIVERSITY, BALTIMORE, MD (US)

(72) Inventors: REBECCA B. SCHULMAN, BALTIMORE, MD (US); DOMINIC SCALISE, BALTIMORE, MD (US)

(21) Appl. No.: 17/271,549

(22) PCT Filed: Aug. 30, 2019
(86) PCT No.: PCT/US2019/049105

§ 371 Date: Feb. 25, 2021

Related U.S. Application Data
(60) Provisional application No. 62/725,596, filed on Aug. 31, 2018.

(51) Int. Cl. C12N 15/113; C07K 14/415

(52) U.S. Cl. CPC C12N 15/113; C12N 2310/16; C07K 14/415

(57) ABSTRACT

The present disclosure provides systems, compositions, and methods related to oligonucleotide buffers for use in generating molecular circuits and their application to DNA nanostructure formation and growth. In particular, the present disclosure provides materials and methods for modulating polynucleotide concentrations using DNA strand-displacement to control molecular reactions for various applications, such as drug delivery, RNA-based therapeutics, chemical synthesis, and nanostructure assembly.

### RELATED APPLICATIONS

This application claims priority to and the benefit of U.S. Provisional Patent Application No. 62/725,596 filed Aug. 31, 2018, which is incorporated herein by reference in its entirety for all purposes.

### GOVERNMENT FUNDING

The invention was made with government support under DE-SC0010595 awarded by the Department of Energy, and NSF-CCF-1161941 and NSF-SHF-1527377 awarded by the National Science Foundation. The government has certain rights to the invention.

### FIELD

The present disclosure provides systems, compositions, and methods related to oligonucleotide buffers for use in generating molecular circuits and their application to DNA nanostructure formation and growth. In particular, the present disclosure provides materials and methods for modulating polynucleotide concentrations using DNA strand-displacement to control molecular reactions for various applications, such as drug delivery, RNA-based therapeutics, chemical synthesis, and nanostructure assembly.

### BACKGROUND

In recent years, a diverse set of mechanisms have been developed that enable DNA strands with specific sequences to sense information in their environment, and to act as directives to control material assembly, disassembly and reconfiguration. Information encoded in sequences of DNA can serve as the inputs and outputs for DNA computing circuits, enabling DNA circuits to act as chemical information processors to program complex behavior in chemical and material systems. Interfaces exist that can release strands of DNA in response to chemical signals, specific wavelengths of light, pH or electrical signals, and DNA strands can direct the self-assembly and dynamic reconfiguration of DNA nanostructures, regulate particle assemblies, control encapsulation, and manipulate materials including DNA crystals, hydrogels, and vesicles. These classes of interface have the potential to enable chemical circuits to exert algorithmic control over responsive materials.

In addition, molecular self-assembly based on DNA displacement is a powerful technology that could enable the bottom-up construction of complex structures such as those seen in biology. Self-assembly often proceeds via crystallization where monomers nucleate and grow above a critical monomer concentration. The chemical potential of the crystallization process is determined by how far the monomer concentration is above the critical concentration for growth. At monomer concentrations well above the critical concentration, nucleation and growth will occur simultaneously. It is often desired to grow crystals from nucleating seeds to control the identity and purity, quantity, and location of the final products. However, there is a narrow range of monomer concentrations in which isothermal growth from nucleating seeds is favored without additional homogenous nucleation, especially in the case of simple 1D crystals. As crystals grow, monomer depletion reduces the chemical potential for growth and in batch reactions growth will eventually stop when the monomer concentration reaches the critical concentration. The higher the seed concentration used, the faster monomer depletion occurs. Thus, monomer depletion limits the lifetime of active growth and the range of conditions over which growth can occur. Monomer depletion can be circumvented with continuous flow reactors that continually replenish monomers to maintain a constant chemical potential for crystal growth. However, physical addition of monomers is not always practical, such as in reactions occurring inside vesicles or artificial cells so other mechanisms of regulation may be desired.

Self-assembly in biology is sustained through chemical regulation of monomer concentrations. For example, the concentrations of self-assembling tubulin monomers are tightly regulated by the cell during microtubule growth. Cells control the flow of chemical energy to produce and degrade tubulin monomers in order to maintain homeostasis. Regulation of tubulin concentrations allows cells to continuously sustain microtubule growth and respond to changes in growth demand, such as an increased number of active microtubule organizing centers during cell division or migration. Since active growth is continuously sustained, cells can build complex hierarchical cytoskeletal structures by sequentially activating branch points that serve as new growth sites. These dynamic growth capabilities would be difficult to implement in a batch crystallization process where monomer depletion limits the active growth time. Such behaviors could be obtained in a continuous flow reactor, but some sort of feedback from the growth process would be required to adjust flow rates or monomer concentrations in the inlet stream to meet changes in growth demand.

### SUMMARY

Embodiments of the present disclosure includes composition for modulating concentration of a polynucleotide. In accordance with these embodiments, the composition includes a source complex comprising a single-stranded target polynucleotide, and a single-stranded initiator polynucleotide capable of associating with the source complex to displace the target polynucleotide from the source complex, wherein the concentration of the target polynucleotide is modulated by altering the concentrations of at least one of the source complex or the initiator polynucleotide.

In some embodiments, the composition further includes a sink complex, wherein the sink complex includes a double-stranded polynucleotide comprising the single-stranded initiator polynucleotide and a complementary single-stranded polynucleotide, wherein the complementary single-stranded polynucleotide is at least partially complementary to both the target polynucleotide and the initiator polynucleotide.

In some embodiments, the source complex includes a double-stranded polynucleotide comprising the single-stranded target polynucleotide and a complementary single-stranded polynucleotide, wherein the complementary single-stranded polynucleotide is at least partially complementary to both the target polynucleotide and the initiator polynucleotide.

In some embodiments, the initiator polynucleotide is at a concentration ranging from about 100 nM to about 1 mM.

In some embodiments, the double-stranded polynucleotide comprising the single-stranded initiator polynucleotide and a complementary single-stranded polynucleotide is at a concentration ranging from about 100 nM to about 1 mM.

In some embodiments, the double-stranded polynucleotide comprising the single-stranded target polynucleotide and a complementary single-stranded polynucleotide is at a concentration ranging from about 100 nM to about 1 mM.

In some embodiments, the concentration of the initiator polynucleotide, the concentration of the double-stranded polynucleotide comprising the single-stranded initiator polynucleotide and a complementary single-stranded polynucleotide, and the concentration of the double-stranded polynucleotide comprising the single-stranded target polynucleotide and a complementary single-stranded polynucleotide are higher than the concentration of the single-stranded target polynucleotide.

In some embodiments, the target polynucleotide comprises from about 10 to about 100 nucleotides.

In some embodiments, the initiator polynucleotide comprises from about 10 to about 100 nucleotides.

In some embodiments, the target polynucleotide and the initiator polynucleotide include at least one toehold domain.

In some embodiments, the toehold domain comprises from about 0 to about 7 nucleotides.

In some embodiments, the composition further includes a reporter complex comprising a reporter molecule.

In some embodiments, the reporter complex includes a double-stranded polynucleotide comprising a single-stranded reporter polynucleotide and a complementary single-stranded quencher polynucleotide, wherein the reporter polynucleotide is at least partially complementary to both the quencher polynucleotide and the target polynucleotide.

In some embodiments, the reporter molecule is selected from the group consisting of a bioluminescent agent, a chemiluminescent agent, a chromogenic agent, a fluorogenic agent, an enzymatic agent and combinations or derivatives thereof.

In some embodiments, the reporter polynucleotide comprises from about 10 to about 100 nucleotides.

In some embodiments, the composition further includes a competitor complex.

In some embodiments, the competitor complex includes a double-stranded polynucleotide comprising a first single-stranded competitor polynucleotide and a second complementary single-stranded competitor polynucleotide, wherein the first competitor polynucleotide is at least partially complementary to both the second competitor polynucleotide and the target polynucleotide.

In some embodiments, the competitor polynucleotide comprises from about 10 to about 100 nucleotides.

In some embodiments, the target polynucleotide and the initiator polynucleotide include at least one of a DNA molecule, an RNA molecule, a modified nucleic acid, or a combination thereof.

Embodiments of the present disclosure also include a method of modulating concentration of a polynucleotide. In accordance with these embodiments, the method includes formulating a composition comprising a source complex comprising a single-stranded target polynucleotide and a single-stranded initiator polynucleotide capable of associating with the source complex to displace the target polynucleotide from the source complex, and increasing or decreasing the concentration of the initiator polynucleotide in the composition to modulate the concentration of the target polynucleotide.

In some embodiments, the method further includes a sink complex, wherein the sink complex comprises a double-stranded polynucleotide comprising the single-stranded initiator polynucleotide and a complementary single-stranded polynucleotide, wherein the complementary single-stranded polynucleotide is at least partially complementary to both the target polynucleotide and the initiator polynucleotide.

In some embodiments, the source complex comprises a double-stranded polynucleotide comprising the single-stranded target polynucleotide and a complementary single-stranded polynucleotide, wherein the complementary single-stranded polynucleotide is at least partially complementary to both the target polynucleotide and the initiator polynucleotide.

In some embodiments, the method further includes a reporter complex comprising a reporter molecule, wherein the reporter complex comprises a double-stranded polynucleotide comprising a single-stranded reporter polynucleotide and a complementary single-stranded quencher polynucleotide, wherein the reporter polynucleotide is at least partially complementary to both the quencher polynucleotide and the target polynucleotide.

In some embodiments, the method further includes a competitor complex, wherein the competitor complex comprises a double-stranded polynucleotide comprising a first single-stranded competitor polynucleotide and a second complementary single-stranded competitor polynucleotide, wherein the first competitor polynucleotide is at least partially complementary to both the second competitor polynucleotide and the target polynucleotide.

In some embodiments, the modulation of the target polynucleotide comprises increasing the concentration of the target polynucleotide, wherein the target polynucleotide displaces a small molecule target bound to an aptamer by binding to at least a portion of the aptamer.

In some embodiments, the modulation of the target polynucleotide comprises increasing the concentration of the target polynucleotide, wherein the target polynucleotide alters one or more conformation properties of a nucleic acid-based hydrogel.

Embodiments of the present disclosure also include a system for modulating concentration of two or more polynucleotides. In accordance with these embodiments, the system includes a first composition comprising a first source complex comprising a first single-stranded target polynucleotide and a first single-stranded initiator polynucleotide capable of associating with the first source complex to displace the first target polynucleotide from the first source complex, and at least a second composition comprising a second source complex comprising a second single-stranded target polynucleotide and a second single-stranded initiator polynucleotide capable of associating with the second source complex to displace the second target polynucleotide from the second source complex, wherein the concentrations of the first and second target polynucleotides are modulated independently within the system by altering the concentrations of at least one of the first and second source complexes or the first and second initiator polynucleotides.

Embodiments of the present disclosure also include a composition for modulating DNA nanostructure formation and growth. In accordance with these embodiments, the composition includes a first source complex comprising two or more single-stranded polynucleotides, a second source complex comprising two or more single-stranded polynucleotides, and an initiator complex comprising two or more single-stranded polynucleotides. In some embodiments, the initiator complex is capable of associating with the first and/or second source complex to displace a polynucleotide from the first or the second source complex. In some embodiments, the growth of the nanotube is modulated by altering a concentration of a polynucleotide of at least one of the first and/or second source complex or the initiator complex.

Embodiments of the present disclosure also include a system for modulating DNA nanostructure formation and growth by modulating the concentration of one or more nanostructure monomers. In accordance with these embodiments, the system includes a first composition comprising a first source complex that is a first inactive nanostructure monomer comprising two or more single-stranded polynucleotides, a first sink complex comprising one or more single-stranded polynucleotides, and a first initiator complex comprising two or more single-stranded polynucleotides that is capable of associating with the first source complex to produce a first active nanostructure monomer. The system also includes at least a second composition comprising a second source complex that is a second inactive nanostructure monomer comprising two or more single-stranded polynucleotides, a second sink complex comprising one or more single-stranded polynucleotides, and a second initiator complex comprising two or more single-stranded polynucleotides that is capable of associating with the second source complex to produce a second active nanostructure monomer. In some embodiments, the growth of the nanostructure is modulated by independently altering the concentration the first and second active monomers by changing the concentrations of at least one of the first and second source complexes the first and second initiator complexes or the first and second sink complexes.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1. A representative schematic diagram of DNA oligonucleotide buffers that can be used to regulate the concentration of a target sequence (X) of DNA (blue) that is initially sequestered within a source complex. Source dissociates in the presence of an initiator strand to release the target sequence and a conjugate sink complex.

FIGS. 2A-2C. Resisting disturbances. When X is added to or removed from an unbuffered solution, its change in concentration is exactly equal to the amount of X added or removed (yellow, slope=1). In contrast, a buffer solution with the form in Eqn 3 (Appendix A) can absorb some of the disturbance to prevent [X] from changing as steeply (cyan, with K_{eq}=0.026). For large enough disturbances the buffer is overwhelmed, at which point the slope again approaches 1 (FIG. 2A). For small disturbances, the slope is approximately linear and is much less than 1 (FIG. 2B). The buffer capacity is the amount of disturbance that can be absorbed while maintaining [X] within a target range (e.g., a range of ±10% of [X]_{eq}). The capacity is proportional to the total concentration of reactants, and its proportionality coefficients c⁺ and c⁻ are functions of the relative reactant concentrations (Eqns 10-11 in Appendix A) (FIG. 2C).

FIGS. 3A-3C. A DNA strand-displacement buffer circuit that regulates the concentration of a target DNA strand X. X is initially bound within a source complex. Source reacts reversibly with initiator to release X, also creating a sink molecule, which drives the reverse reaction (FIG. 3A). The concentration of X is monitored by a reporter complex. X reacts reversibly with reporter to separate a quencher-fluorophore pair, increasing the intensity of fluorescence. When sequestered in the source complex, the first toehold domain (black) on X is not available to initiate reactions with the reporter (FIG. 3B). A competitor complex (commonly used as a “threshold” in other strand-displacement literature²⁵) can irreversibly bind and sequester X via a fast 7 nt toehold, reducing its free concentration in solution. A “leakless” architecture was used to suppress reactions between species not designed to react (FIG. 3C).

FIGS. 4A-4C. Concentration parameter space showing equilibration of [X] with varied concentrations of [S]₀, [I]₀, and [N]₀. Experimental data (solid lines) showing approach to equilibrium. Exponential fits shown as dashed lines (FIG. 4A). Equilibrium concentration vs. [S]₀=[I]₀. Cyan points correspond to the [S]₀=[I]₀=8 μM trajectories from panel (a), yellow points to yellow trajectories, and red to red (FIG. 4B). Relaxation time constants vs. [N]₀. Error bars here and elsewhere depict 95% confidence intervals (1.96σ/\sqrt{n}) (FIG. 4C).

FIGS. 5A-5E. Response of the oligonucleotide buffer to disturbances. An 8 μM uniform buffer for target species X disturbed with additions of 50 nM excess X every three hours. Differences in the amount of time required to add and mix the disturbances into the wells (during which no measurements were made) of a 96-well plate cause differences between the peak amplitudes of the disturbances. (FIG. 5A) Since the fastest changes in concentration occur immediately after the disturbance is added, the first measured value of [X] is highly dependent on this delay time. Addition of 50 nM X disturbances to a solution containing no buffering reaction, showing cumulative increase in concentration (FIG. 5B). The change in equilibrium concentration of X vs. the total concentration of X added as disturbance for the buffered data in panel (a) (FIG. 5C). An 8 μM buffer disturbed with an addition of 100 nM competitor, C, which consumes X (FIG. 5D). An 8 μM buffer disturbed with large pulses of 250 nM X, followed by 250 nM competitor (FIG. 5E).

FIGS. 6A-6C. Buffers for different oligonucleotides operate in tandem without crosstalk. Uniform 8 μM buffer for a second sequence X₂ before and after a 50 nM addition of X₂. The reporter for X₂ uses a HEX fluorophore (FIG. 6A). Uniform 8 μM buffer for X before and after a 50 nM addition of X (FIG. 6B). (Using the reporter in FIG. 2c with FAM fluorophore). The concentrations of X and X₂ in a solution containing 8 μM uniform buffers (and associated reporters) for both sequences, with 50 nM disturbances added at the same times as (a) and (b) (FIG. 6C).

FIG. 7. Faster responses to disturbances by a buffer with longer toeholds. A 1 nt source toehold and a 4 nt sink toehold were used to increase the rate of response of a uniform 8 μM fast buffer. X was added to disturb the system at times noted.

FIG. 8. Detailed diagram for 0-nucleotide reaction between the slow Source and slow Initiator, with a 0 nucleotide toehold (no toehold) on the forward reaction. The reaction is initiated when the end base pair on the Source complex frays open, effectively creating a transient 1-nucleotide “toehold” for the Initiator to bind. Branch migration and displacement of the signal strand X then proceeds as usual for an ordinary toehold-mediated strand-displacement reaction. Signal X and sink N are produced, and the reaction is reversible.

FIG. 9. Fast buffer reaction diagram showing the 1-nucleotide toehold on Source (FAST) that drives the forward reaction shown here faster than the forward reaction (FIG. 3A), and the 4-nucleotide Sink (FAST) toehold that drives the reverse reaction shown here faster than the comparable reverse reaction (FIG. 3A). Making kₛᵢₙₖ faster decreases the response time of the buffer.

FIGS. 10A-10B. Reporter Calibrations. Full complement calibration to convert from raw fluorescence in counts per second to the concentration of unquenched fluorophore [R_{F}] in solution (FIG. 10A). Reverse calibration to convert from [R_{F}] to [X] (FIG. 10B).

FIGS. 11A-11C. Comparison between reporters with different toehold lengths, using a simple bimolecular model to simulate the kinetics. For both simulations an 8 μM uniform buffer was used with a 0 nt source toehold and a 2 nt sink toehold, with [Reporter]₀=[R_{Q}]₀=100 nM. A reporter with 5 nt toeholds correctly recapitulates the kinetics of the buffering reaction. This is the reporter selected for various experiments described herein (FIG. 11A). A slower reporter with 2 nt toeholds is not fast enough to recapitulate the kinetics of the buffering reaction (FIG. 11B). The fast buffer (with 0 nt source toehold, and 2 nt sink toehold) equilibrates faster than the 5 nt reporter indicates it does (FIG. 11C).

FIGS. 12A-12D. Three-step model^{S1-R4} of a DNA strand-displacement reaction, in which an input or invader strand displaces an incumbent or output strand from a complex. Invader is single-stranded, while the incumbent strand is initially hybridized to the complex (FIG. 12A). Invader binds to the complex via a short unstable “toehold” domain (black, t), which initiates the displacement reaction (FIG. 12B). In a random walk process, the invader strand competes with the incumbent strand to occupy the longer “recognition” domain (cyan, 1) (FIG. 12C). The incumbent strand, now bound only by the short unstable toehold domain, dissociates from the complex (FIG. 12D). All steps in the reaction are reversible. The lengths of the toehold domains determine the rate constants with which strands dissociate from the complex when bound only by the toehold. These rate constants determine the effective rate constant of the entire strand-displacement reaction.

FIG. 13. Three-step model predictions of buffer kinetics with different concentrations of Source, Initiator, and Sink. Blue lines correspond to buffers with [S]₀=[I]₀=8 uM, yellow to [S]₀=[I]₀=4 uM, and red to [S]₀=[I]₀=2 uM. [Reporter]=100 nM, with [R_{Q}]=100 nM.

FIGS. 14A-14B. Three-Step Model Predictions of the relaxation time constant with and without the reporter present. Time constants with no reporter, showing no significant dependence of the time constant on the concentrations of Source and Initiator (FIG. 14A). Time constants with reporter included ([Reporter]₀=100 nM, with [R_{Q}]₀=100 nM), showing dependence of time constant on the initial concentrations of Source and Initiator (FIG. 14B). Note: toehold occlusion effects are not included in these simulations.

FIGS. 15A-15D. The effect of toehold occlusion on buffers with negative disturbances. DNA strand-displacement diagram illustrating the Initiator occluding the Competitor's toehold (FIG. 15A). Three-step model simulation of a buffer responding to a negative disturbance of [C]=100 nM without any occlusion (initial concentrations at [S]=[I]=[N]=8 uM, [Reporter]=100 nM, [R_{Q}]=100 nM) (FIG. 15B). The same simulation as in b, with occlusion effects included between I+Reporter, I+R_{F}, and I+N. The equilibrium concentration is reduced (FIG. 15C). The same simulation as in c, with the occlusion of I+C included (FIG. 15D). The negative disturbance is damped, as observed experimentally in FIG. 5A. This occlusion effectively reduces the reaction rate constant between X and the competitor C, which prevents the disturbance species from reacting quickly to consume X.

FIG. 16. Time constants as a function of total disturbance of X added to an 8 uM uniform buffer fit to data from FIG. 5A, in which a 50 nM disturbance is repeatedly added to the system and then allowed to equilibrate. The time constant to relax to equilibrium does not vary significantly for the range of disturbances tested.

FIGS. 17A-17B. Negative disturbances to the 8 μM uniform fast buffer with additions of competitor as noted in the figure. A zoomed in plot showing the rapid response to the disturbances (FIG. 17A), and a zoomed out plot showing the gradual consumption of competitor, after which the system recovers to equilibrium (FIG. 17B).

FIG. 18. A representative sequential release circuit. At each stage, an output molecule is released, and then the next stage is triggered. The red stage triggers the cyan stage, which triggers the green stage, etc.

FIGS. 19A-19B. Asynchronous Sequential Release. The reaction cascade consists of stages of Payload and Convert complexes (see FIG. 10). At each stage, a Trigger molecule first reacts quickly with the Payload to release a fluorescent Output into solution. Any remaining Trigger then reacts slowly with the Convert complex, which converts it into the Trigger molecule for the next stage (FIG. 19A (SEQ ID NOs: 1, 227-236)). Experimental data showing the fluorescent Outputs being released in order, with 25 nM Payloads and 37.5·(4−i)nM Convert_{i,i+1}. Leakless architecture was used to prevent some unintended leak reactions between Convert_{i-,i+1} and Payloadᵢ complexes (FIG. 19B).

FIGS. 20A-20C. Clocking. Clock production DSD circuit (FIG. 20A (SEQ ID NOs: 1, 3, and 237)). Experimental data showing the production circuit releasing Trigger₁, with 1 μM Source and Initiator, without a downstream sequential release cascade (FIG. 20B). The sequential release cascade connected to the production circuit with 1 μM Source and Initiator, 25 nM Payloads, and 37.5·(4−i)nM Convert_{i,i+1} (FIG. 20C). The timing of release events is now rate limited by the production circuit, making the delay times between stages roughly linear.

FIGS. 21A-21C. Branching. DSD diagram for a conditional Convert_{1,2A} complex, which is active in the presence of an associated Deprotect strand (see sequences for parallel Convert_{1,2B} system in FIG. 9). Experimental results for a branched two-stage sequential release program, with branch 2A deprotected (FIG. 21B). Experimental results for a branched two-stage sequential release program, with branch Bb activated (FIG. 21C). For both FIGS. 21A-21B (SEQ ID NOs: 229 and 238-240), 1 μM Source and Initiator was used, 25 nM Payloads, 37.5 nM Convert complexes, and 50 nM of the stated Deprotect strands.

FIG. 22. Hydrogel-based drug delivery. Representative schematic diagrams of the use of the oligonucleotide buffer compositions and systems disclosed herein within a drug deliver matrix (e.g., hydrogel).

FIGS. 23A-23D. Seeded DNA nanotube design and growth. FIG. 23A, DNA tile design. Left: DNA nanotubes are composed of monomers, termed DNA tiles that are composed of five strands of DNA that fold into a double crossover structure with single stranded sticky ends. Right: complementarity between tile sticky ends program the tiles to self-assemble into a specific lattice that cyclizes to become a nanotube. FIG. 23B, DNA origami seed design. The seed is a cylindrical DNA origami folded from single-stranded M13 bacteriophage DNA. One face of the seed is bound to DNA tile adapter strands that present one set of sticky ends. There are six tile adapters around the circumference of the seed providing a stable nucleus for nanotube growth (inset). FIG. 23C, DNA nanotube growth in different tile concentration regimes. Left: plot of the free tile concentrations during nanotube growth initiated in the unseeded (light blue), seeded (blue), or no (dark blue) growth regimes. The DNA origami seed has a slight nucleation barrier, so the minimum tile concentration required to nucleate nanotubes from seeds is slightly higher than the critical tile concentration for nanotube growth (dashed line). Right: schematics of nanotube growth in the different growth regimes. Fluorescence micrographs depict nanotubes (green) and seeds (red) after 24 hours of growth at different tile concentrations. The 1000 nM tiles sample did not contain seeds. Scale bars: 10 μm. d, Schematic of an ideal nanotube growth system in which feedback control maintains a specific free tile concentration by replenishing tiles as they are depleted from growth.

FIGS. 24A-24D. Characterization of DNA nanotube growth at low and high fixed tile concentrations. FIG. 24A, Fluorescence micrographs of nanotubes and seeds after growth with the specified concentrations of tiles and seeds. Scale bars: 10 μm. FIG. 24B, Mean seeded nanotube lengths during growth with different seed concentrations. Error bars represent 95% confidence intervals from bootstrapping. FIG. 24C, Fractions of seeds with nanotubes (left) and nanotubes with seeds (right) after 72 hours of growth with different seed concentrations. Error bars represent 95% confidence intervals of proportions. FIG. 24D, Histograms of seeded nanotube lengths for the samples in (FIG. 24A).

FIGS. 25A-25C. Stochastic kinetic simulations of seeded nanotube growth with and without tile depletion. FIG. 25A, Reactions of the kinetic model. Tiles can reversibly bind to a growing nanotube (left) or to an open seed face (right). The presence of a nucleation barrier for the seed⁷ was modeled as a higher off rate for tiles bound to a seed than tiles bound to a nanotube. FIGS. 25B, 25C, Simulation results for nanotube growth with (FIG. 25B) or without (FIG. 25C) tile depletion with an initial tile concentration of 150 nM. Only the concentration of the REd tiles is shown for clarity as the SEd tiles follow the same trajectories. For the simulations, k_{ON}=2×10⁵ M⁻¹s⁻¹, Δ_{GT-NT}=−9.3 kcal/mol, k_{OFF, T-S}=6*k_{OFF, T-NT}.

FIGS. 26A-26D. Tile concentration buffering is predicted to resist changes in tile concentrations during nanotube growth. FIG. 26A, Reaction network for tile concentration buffering. Inactive Source complexes (Sᵢ) react with Initiator complexes (Iᵢ) via a strand displacement reaction initiated by a single-stranded toehold domain (TH_{f, i}) to produce active tiles (Tᵢ) and Sink strands (Nᵢ). The active tiles and Sink strands can react via a strand displacement reaction initiated by a single-stranded toehold domain (TH_{r, i}) to reverse the tile production reactions. FIG. 26B, Inactive Source tiles cannot bind to seeds or a growing nanotube face. Incorporation of the active tiles into nanotubes sequesters the toehold domains for the reverse reactions of the reaction network in (FIG. 26A). FIG. 26C, Tile incorporation into nanotubes causes the reaction network to produce more active tiles to resist a change in the equilibrium tile concentration. FIG. 26D, Stochastic kinetic simulation results for nanotube growth with tile concentration buffering. The same parameters from the simulations in FIG. 25 were used here. The forward and reverse buffering reaction rate constants were assumed to be 1×10² M⁻¹s⁻¹ and 1×10⁴ M⁻¹s⁻¹, respectively. The Source and Initiator concentrations were 5.5 μM and Sink strand concentrations were 1.69 μM to set the equilibrium tile concentration to roughly 150 nM. No active tiles were present at the beginning of the simulations.

FIGS. 27A-27E. Nanotube growth is significantly improved with tile concentration buffering compared to growth with 150 nM tiles. FIG. 27A, Fluorescence micrographs of seeded nanotubes during growth with tile concentration buffering (Buffering) or with 150 nM tiles. Scale bars: 10 μm. FIG. 27B, mean seeded nanotube lengths during growth with and without tile concentration buffering at different seed concentrations. Error bars represent 95% confidence intervals from bootstrapping. FIG. 27C, Histograms of seeded nanotube lengths during growth with 0.33 nM seeds. FIG. 27D, Fractions of nanotubes with seeds (top) and seeds with nanotubes (bottom) after growth with different seed concentrations. Error bars represent 95% confidence intervals of proportions. FIG. 27E, mean concentration of tiles incorporated in nanotubes during growth. The 150 nM tiles samples (gray) all converge to 50-70 nM. Tile concentration buffering reactions were conducted as described in the methods.

FIGS. 28A-28D. The tile concentration buffer adapts to changes in growth demand. FIG. 28A, Fluorescence micrographs of seeded nanotubes during growth with 150 nM tiles or with tile concentration buffering (Buffering). S1 seeds (0.1 nM) were present at the beginning of the experiment and after 24 hours S2 seeds (0.1 nM) were added. The S1 and S2 seeds were identical other than being labeled with different fluorescent tags. Scale bars: 10 μm. FIG. 28B, Histograms of seeded nanotube lengths during nanotube growth with tile concentration buffering. FIG. 28C, mean seeded nanotube lengths during growth with and without tile concentration buffering for nanotubes grown from the S seeds (left) or the S2 seeds (right). Error bars represent 95% confidence intervals from bootstrapping. FIG. 28D, Fractions of each seed with nanotubes during nanotube growth. Error bars represent 95% confidence intervals of proportions. Tile concentration buffering reactions were conducted as described further herein.

FIGS. 29A-29G. A stochastic kinetic model recapitulates the experimental results for nanotube growth with tile concentration buffering. FIGS. 29A-29C, Comparison of experimental results to stochastic simulations for mean seeded nanotube lengths (FIG. 29A), fraction of seeds with nanotubes (FIG. 29B), mean concentration of tiles incorporated into nanotubes (FIG. 29C), and nanotube length distributions (FIG. 29D) during nanotube growth with tile concentration buffering (dashed lines represent simulation results). FIGS. 29E-29G, Simulations of tile concentration buffering species during nanotube growth. Only the REd buffering species are shown as the SEd species follow the same trajectories. Solid lines represent the theoretical equilibrium values of the buffering species during the course of the reaction. Stochastic simulations were conducted using a k_{f} of 1×10 M⁻¹s⁻¹ and a kᵣ of 1×10² M⁻¹s⁻¹ for the buffering reaction rate constants and the concentrations of the buffering species described in the methods.

FIGS. 30A-30D. With a fixed 1000 nM of tiles, nanotube growth produces similar results with and without seeds. FIGS. 30A-30C, Fluorescence micrographs of 1000 nM tiles grown without and with 0.1 nM (FIG. 30A), 0.33 nM (FIG. 30B), 1 nM (FIG. 30C) seeds. Scale bars: 10 μm. Samples without seeds were imaged at the same dilutions as corresponding samples with seeds. Histograms of nanotube lengths are shown below the fluorescence micrographs. The same number of nanotubes nucleate, the mean nanotube lengths are the similar, and similar broad length distributions are observed for nanotubes growth both with and without seeds.

FIGS. 31A-31D. A stochastic kinetic model recapitulates the experimental results for nanotube growth with 150 nM tiles (see FIG. 24). FIGS. 31A-31C, Comparison of experimental results to stochastic simulations for mean seeded nanotube lengths (FIG. 31A), fraction of seeds with nanotubes (FIG. 31B), mean concentration of tiles incorporated into nanotubes (FIG. 31C), and nanotube length distributions (FIG. 31D) during nanotube growth with 150 nM tiles (dashed lines represent simulation results). Stochastic simulations conducted with 250 seeds using: k_{ON}=2×10⁵ M⁻¹s⁻¹ k_{OFF, T-S}=6*k_{OFF, T-NT}, ΔG=−9.3 kcal/mol.

FIGS. 32A-32E. Nanotube growth with buffering for different tile concentration setpoints. All reactions were conducted with an initial concentration of 5.5 μM for the Source and Initiator complexes and the concentrations of the Sink strands were varied (FIG. 32A). FIG. 32A, Table of the initial concentrations of the Sink strands (N) and the theoretical equilibrium tile concentration for the initial conditions (calculated as described herein). FIGS. 32B-32E, Quantification of mean seeded nanotube lengths (FIG. 32B), mean concentration of tiles incorporated into nanotubes (FIG. 32C), fraction of nanotubes with seeds (FIG. 32D), and fraction of seeds with nanotubes (FIG. 32E) for nanotube growth with the tile concentration buffering conditions described in (FIG. 32A) at three different seed concentrations. Sink concentrations of 1 μM and 1.25 μM produce similar growth results and increasing the Sink concentration further results in less growth.

FIG. 33. Fluorescence micrographs of nanotube growth with buffering in the absence of seeds. These experiments were conducted alongside the experiments in FIG. 32. Other than the 1 μM Sink sample there is not much nanotube growth without seeds. All images taken after a 100× dilution of the sample (the same dilution used to image the 0.1 nM seed samples in FIG. 32). Scale bars: 10 μm.

FIGS. 34A-34F. Mean nanotube length quantification can be skewed for samples with long nanotubes as longer nanotubes are prone to breaking during imaging. FIGS. 34A-34C, Quantification of mean seeded nanotube lengths (FIG. 34A), fraction of nanotubes with seeds (FIG. 34B), and nanotube length distributions (FIG. 34C) during nanotube growth with buffering using 5.5 μM of Source and Initiator complexes and 1.25 μM Sink strands. In the 0.1 nM sample, after 48 hrs when the nanotubes are roughly 10 μm long, there is a significant drop in the fraction of nanotubes with seeds (FIG. 34B) and shorter nanotubes start to arise in the length distributions (FIG. 34C). It was observed that the long nanotubes in these samples were prone to breaking as they attached to coverslip surface during imaging. Nanotubes breaking results in an increase in the number of nanotubes without seeds and a broadening in the length distributions. Given hardly any growth is observed with tile concentration in the absence of seeds (FIG. 33) these results are not consistent with unseeded nanotube growth over the course of the experiments. Nanotubes breaking during imaging skews the quantification of mean seeded nanotube length, resulting in shorter average lengths for samples with long nanotubes. For example, looking at the length distributions of the sample with 0.1 nM seeds after 72 hrs (FIG. 34C, top panel), there appears to be two distributions of seeded nanotube lengths, one centered around 18 μm and another between 5-10 μm (where nanotubes are short enough to that they do not break). These results suggest, in the absence of nanotubes breaking, that the actual mean seeded nanotube length after 72 hrs is around 18-20 μm rather than the 11 μm. FIGS. 34D-34F show similar results to (FIGS. 34A-34C) but for nanotubes grown with buffering using 1.5 μM of each Sink strand.

FIGS. 35A-35D. Despite producing similar final results, nanotube growth with buffering proceeds much differently than growth with a fixed 1000 nM tiles and results in much more monodispersed nanotube lengths. FIG. 35A, Histograms of seeded nanotube lengths during growth. FIG. 35B, mean seeded nanotube lengths during growth with and without tile concentration buffering at different seed concentrations. Error bars represent 95% confidence intervals from bootstrapping. FIG. 35C, mean concentration of tiles incorporated in nanotubes during growth. d, Fractions of seeds with nanotubes (left) and nanotubes with seeds (right) after 72 hours of growth with different seed concentrations. Error bars represent 95% confidence intervals of proportions.

FIGS. 36A-36E. Nanotube growth with a fixed 1000 nM tiles does not sustain active growth like buffering. FIG. 36A, Fluorescence micrographs of seeded nanotubes during growth with a fixed 1000 nM tiles or with tile concentration buffering (Buffering). S1 seeds (0.1 nM) were present at the beginning of the experiment and after 24 hours S2 seeds (0.1 nM) were added. The S1 and S2 seeds were identical other than being labeled with different fluorescent tags. Scale bars: 10 μm. FIG. 36B, Histograms of seeded nanotube lengths during nanotube growth with a fixed 1000 nM tiles. The distributions for the S1 and S2 seeds are very similar indicating the S2 seeds attached to nanotubes are likely the results of seeds joining to preexisting tubes rather than seeds nucleating growth. FIG. 36C, mean seeded nanotube lengths during growth with and without tile concentration buffering for nanotubes grown from the S5 seeds (left) or the S2 seeds (right). Growth stops after 8 hours for the 1000 nM tiles samples on both seeds, further supporting that the nanotubes attached to S2 seeds are the result of joining rather than active growth. Error bars represent 95% confidence intervals from bootstrapping. FIG. 36D, Fraction of seeds with nanotubes during nanotube growth. Error bars represent 95% confidence intervals of proportions.

FIG. 36E, Fraction of nanotubes with seeds during nanotube growth, showing significant unseeded growth for the 1000 nM tiles sample. These fractions were computed based on the total number of seeded nanotubes for both seeds so there is an increase in the number of nanotubes with seeds after 24 hours due to the S2 seeds joining to unseeded nanotubes. Tile concentration buffering reactions were conducted as described in the methods provided herein.

FIGS. 37A-37E. FIGS. 37A-37C, Comparison of stochastic simulations to experimental results of nanotube growth with tile concentration buffering for mean seeded nanotube lengths, fraction of seeds with nanotubes, mean concentration of tiles incorporated into nanotubes. Simulations in (FIG. 37A), (FIG. 37B), and (FIG. 37C) were conducted with different values of k_{ON}. FIG. 37D-37F, Tile concentrations during nanotube growth in the simulations in (FIG. 37A), (FIG. 37B), and (FIG. 37C), respectively. Only the R tiles are shown as the S tiles follow the same trajectories. Solid lines represent the theoretical equilibrium values of the buffering species during the course of the reaction. Stochastic simulations were conducted with 250 seeds using a k_{f} of 1×10² M⁻¹s⁻¹ and a kᵣ of 1×10⁴ M⁻¹s⁻¹ for the buffering reaction rate constants and the buffering species concentrations described in the methods provided herein.

FIGS. 38A-38F. FIGS. 38A-38C, Comparison of stochastic simulations to experimental results of nanotube growth with tile concentration buffering for mean seeded nanotube lengths, fraction of seeds with nanotubes, mean concentration of tiles incorporated into nanotubes. Simulations in (FIG. 38A), (FIG. 38B), and (FIG. 38C) were conducted with different values of k_{ON}. FIGS. 38A-38C, Tile concentrations during nanotube growth in the simulations in (FIG. 38A), (FIG. 38B), and (FIG. 38C), respectively. Only the R tiles are shown as the S tiles follow the same trajectories. Solid lines represent the theoretical equilibrium values of the buffering species during the course of the reaction. Stochastic simulations were conducted with 250 seeds using a k_{f} of 1×10⁰ M⁻¹s⁻¹ and a kᵣ of 1×10² M⁻¹s⁻¹ for the buffering reaction rate constants and the buffering species concentrations described in the methods provided herein. The results of (FIG. 38B) are presented in the FIG. 29.

FIGS. 39A-39H. FIGS. 39A-39D, Comparison of stochastic simulations to experimental results of nanotube growth with tile concentration buffering for mean seeded nanotube lengths, fraction of seeds with nanotubes, mean concentration of tiles incorporated into nanotubes. Simulations in (FIG. 39A), (FIG. 39B), (FIG. 39C), and (FIG. 39D) were conducted with different concentrations of the Sink strands. FIGS. 39D-39F, Tile concentrations during nanotube growth in the simulations in (FIG. 39A), (FIG. 39B), (FIG. 39C), and (FIG. 39D), respectively. Only the R tiles are shown as the S tiles follow the same trajectories. Solid lines represent the theoretical equilibrium values of the buffering species during the course of the reaction. Stochastic simulations were conducted with 250 seeds using a k_{f} of 1×10⁰ M⁻¹s⁻¹ and a kᵣ of 1×10² M⁻¹s⁻¹ for the buffering reaction rate constants and a k_{ON} of 2×10⁵ M⁻¹s⁻¹ for tile attachment. The Source and Initiator complexes were at 5.5 μM in all the simulations.

FIGS. 40A-40C. Stochastic simulations of nanotube growth with buffering show increasing the concentrations of buffering components should increase nanotube growth capacity. Compared to the experimental conditions used in FIG. 27 (FIG. 40A), the Source and Initiator complex concentrations were increased 2-fold (FIG. 40B) and 5-fold (FIG. 40C) and the Sink concentration was changed to obtain the same predicted initial active tile equilibrium value as in (FIG. 40A). Increasing the concentrations of the tile buffering components results in increased growth capacity. As the concentration of Source, Initiator, and Sink are increased, the rate at which the active tile concentration drops over the course of the experiments decreases and the total amount of tiles incorporated into nanotubes increases, particularly for the 1 nM seed samples. Stochastic simulations were conducted with 250 seeds for (FIG. 40A) and 100 seeds for (FIGS. 40B, 40C).

FIGS. 41A-41C. Source complexes negatively affect nanotube growth. FIG. 41A, Fluorescence micrographs of nanotubes grown with a fixed 50 nM of tiles and increasing concentrations of the Source complexes. Nanotubes were grown with 10 μM seeds and images were taken after 24 hours of growth. Scale bars: 10 μm. In these experiments, T_{R}-3 and T_{S}-3 were modified with Cy3 on their 5′ ends and an unmodified T_{S}-2 strand was used: 5′TCTGGTAGAGCACCACTGAGAGGT. FIG. 41B, 41C, Reversible interactions between the Source complexes and the active tiles. These reactions could lower the effective active tile concentration by blocking one of the sticky end strands that binds to a nanotube growth face. The reactions shown in (FIG. 41B) should not influence the buffering reactions as they do not block any of the toeholds used for buffering. The reactions shown in (FIG. 41C) could influence the buffering reactions because they block the toehold for the Tile-Sink reaction. These interactions effectively remove active tiles from the buffering reaction network, so the buffer may compensate for these interactions by shifting the equilibrium to produce more tiles.

FIGS. 42A-42C. Initiator complexes negatively affect nanotube growth. FIG. 42A, Fluorescence micrographs of nanotubes grown with a fixed 50 nM of tiles and increasing concentrations of the Initiator complexes. Nanotubes were grown with 10 μM seeds and images were taken after 24 hours of growth. Scale bars: 10 μm. In these experiments the Initiator complexes were composed of slightly different sequences, denoted with a d prefix: dN_{R}: 5′GTATGCATCTGTCCCTAG, dT_{R}-2: 5′CTAGGGACAGATGCATACCGGCAT, dN_{S}: 5′TTGATCCTTAAGCGGTTG, dT_{S}-2: 5′TTCAACCGCTTAAGGATCAAAGAGGT. Further, T_{R}-3 and T_{S}-3 were modified with Cy3 on their 5′ ends and an unmodified T_{S}-2 strand was used: 5′ TCTGGTAGAGCACCACTGAGAGGT. FIG. 42B, Reversible interactions between the Initiator complexes and the growth face of a DNA nanotube or a seed face. The Initiator can reversibly block growth sites, potentially slowing down growth or possibly preventing it if present at high enough concentrations.

FIGS. 43A-43D. Representative images from the MATLAB image analysis algorithm. FIG. 43A: the overlayed fluorescence micrographs of the image to be processed. Nanotubes are green and seeds are red. FIG. 43B: the overlayed binary image output that was analyzed by MATLAB to quantify the fraction of seeds with nanotubes. The blue squares indicate the seeds that the algorithm identified as being attached to a nanotube. FIG. 43: the overlayed binary image output that was analyzed by MATLAB to quantify the fraction of nanotubes with seeds. Note that nanotubes at the edge of the image for (FIG. 43B) have been removed. The blue squares indicate the nanotube endpoints that the algorithm identified as being attached to a seed. FIG. 43D: the overlayed binary image output that was analyzed by MATLAB to quantify nanotube length. Note the branched objects and unseeded nanotubes from the image in (FIG. 43C) have been removed.

FIG. 44. DNA tile and tile buffering components. T_{R} and S_{R} show SEQ ID NOs: 17-21. T_{S} and S_{S} show SEQ ID NOs: 22-26.

FIG. 45. Representative of a seed composed of a scaffold strand (M13mp18 DNA (7,240 bases)), 72 staple strands, and 24 adapter strands (strands on the adapters that possess the tile sticky end sequences). The dark staples direct the structure to cyclize into a cylinder.

FIG. 46. Representative depiction of six tile adapters around the circumference of a seed face (AS1 (SEQ ID NOs: 101-104, AS2 (SEQ ID NOs: 105-108), AS3 (SEQ ID Nos: 109-112), AS4 (SEQ ID NOs: 113-116), AS5 (SEQ ID NOs: 117-120), and AS6 (SEQ ID NOs: 121-124)), three adapters that present the T_{R} sticky ends and three adapters that present the T_{S} sticky ends. The adapters resemble DNA tiles composed but are bound to M13 DNA (gray) where strand 2 would normally be on the tiles.

### DETAILED DESCRIPTION

Described herein are systems, compositions, and methods related to oligonucleotide buffers for use in generating molecular circuits and their application to DNA nanostructure formation and growth. In particular, the present disclosure provides materials and methods for modulating polynucleotide concentrations using DNA strand-displacement to control molecular reactions for various applications, such as drug delivery, RNA-based therapeutics, chemical synthesis, and nanostructure assembly.

Section headings as used in this section and the entire disclosure herein are merely for organizational purposes and are not intended to be limiting.

Appendix A is being filed herewith as part of the present disclosure.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Polynucleotide” as used herein can be single stranded or double stranded, or can contain portions of both double stranded and single stranded sequence. The polynucleotide can be nucleic acid, natural or synthetic, DNA, genomic DNA, eDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Recombinant” when used with reference (e.g., to a cell, or nucleic acid, protein, or vector) indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed, or not expressed at all.

“Sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Additionally, as would be recognized by one of ordinary skill in the art based on the present disclosure, nucleic acid sequence comparisons can be performed and/or defined in terms of Watson-Crick complementarity with other sequences, in contrast to absolute sequence identity. For example, a “Class” of sequences can be defined by a set of domain lengths and what the domains are complementary to (or not complementary to).

“Variant” as used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequence substantially identical thereto.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids. See Kyte et al., J. Mol. Bioi. 1982, 157, 105-132. The hydropathic index of an amino acid is based on a consideration of its hydrophobicity and charge. It is known in the art that amino acids of similar hydropathic indexes can be substituted and still retain protein function. In one aspect, amino acids having hydropathic indices of 2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in polypeptides retaining biological activity, for example immunogenicity, as is understood in the art. Substitutions can be performed with amino acids having hydrophilicity values within 2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be a polynucleotide sequence that is substantially identical over the full length of the full gene sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

As discussed herein, nucleic acid sequence comparisons can be performed and/or defined in terms of Watson-Crick complementarity with other sequences, in contrast to absolute sequence identity. For example, a “variant” of sequences can be defined by a set of domain lengths and what the domains are complementary to (or not complementary to).

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. OLIGONUCLEOTIDE-BASED BUFFERS AND SYSTEMS

Embodiments of the present disclosure demonstrate a DNA strand-displacement reaction for buffering the concentration of oligonucleotides. High concentrations of reactants continuously release and recapture a target strand, forming an equilibrium that resists perturbations to the concentration of the target. Using this architecture, buffer compositions and systems can be designed for arbitrary sequences with a wide range of setpoints, response times and capacities. Several buffers can operate in parallel within the same solution, to independently regulate the concentrations of multiple target strands, as described further below.

In some embodiments, oligonucleotide buffers can be incorporated into a wide variety of existing reactions in which oligonucleotides play a key role, including but not limited to, self-assembly, sensing, photochemistry, and molecular release. Oligonucleotide buffers can also regulate molecules besides DNA when coupled to actions that interface with other species, such as enzymes and small molecules, as described further below.

The ability to maintain the concentrations of molecular species is critical for building large reaction networks that operate reliably for extended times. Biological reaction networks often resist changes in concentration using mechanisms that are mathematically quite similar to buffers. For example, competing processes of synthesis and degradation in gene networks regulate the concentrations of most RNA and proteins. Similar competing reactions on faster time scales also regulate the concentrations of actin and active membrane receptors. The ability to incorporate molecular buffering for a variety of species into synthetic chemical systems can facilitate the design of robust and scalable synthetic chemical reaction networks.

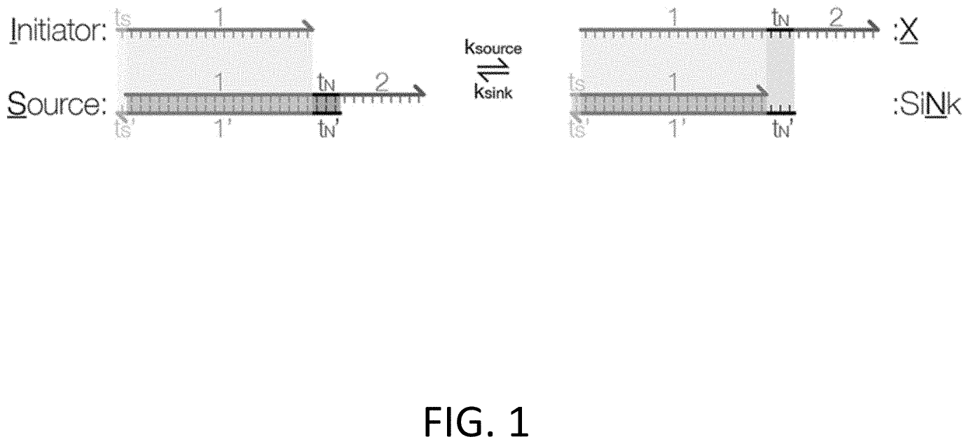

Embodiments of the present disclosure provide a class of buffer compositions and systems that regulate the concentrations of oligonucleotides (FIG. 1), short synthetic sequences of DNA, in an analogous manner to how acid-base buffers regulate pH. Each buffer regulates the concentration of a specific DNA sequence, and multiple different buffers can operate in the same solution, independently controlling the concentrations of their different target sequences. The oligonucleotide buffer compositions and systems of the present disclosure include the use of DNA strand-displacement (DSD) reactions, sequence-specific DNA hybridization processes with tunable kinetics, which have previously been used to implement information processing reactions including amplifiers, neural networks, and Boolean logic circuits. Embodiments of the present disclosure demonstrate how DSD reactions can operate in regimes containing high reactant concentrations and low reaction rate constants. Buffering the concentrations of oligonucleotides could allow for the self-assembly of larger DNA structures or DNA-templated structures with fewer defects by providing a constant supply of fresh monomers, stabilizing the nucleation of DNA crystal structures, and could enable DNA circuits and sensors to operate for extended durations by restoring depleted reactants.

Embodiments of the present disclosure also provide a DNA strand-displacement circuit that releases a series of different Output strands of DNA, one after another. This circuit serves as a simple scheduling program to trigger molecular events at discrete times. In accordance with these embodiments, a four-stage circuit with 25 nM concentrations per stage was developed. The circuit was demonstrated to run in an asynchronous or clocked configuration. For example, in the asynchronous mode, the time delay between stages is non-uniform and slows down dramatically between stages, while the clocked mode enforces more uniform temporal spacing between stages. In some embodiments, the circuit can be modified to enable conditional logic, where different branches of the release program can be activated depending on the presence of activating signal strands in the solution.

Embodiments of the present disclosure include buffered compositions and systems for modulating the concentration of a polynucleotide. In some embodiments, modulation includes driving the concentration of a polynucleotide back toward a predetermined setpoint when exposed to a transient disturbance, loading, or other increases or decreases in concentration. In accordance with these embodiments, the compositions and systems can include a source complex comprising a single-stranded target polynucleotide, or a part of a single-stranded target polynucleotide. The source complex can include a double-stranded polynucleotide comprising the single-stranded target polynucleotide and a complementary single-stranded polynucleotide, wherein the complementary single-stranded polynucleotide is at least partially complementary to both the target polynucleotide and the initiator polynucleotide (FIG. 1B). In some embodiments, the target polynucleotide comprises from about 10 to about 100 nucleotides. In some embodiments, the target polynucleotide comprises from about 10 to about 90 nucleotides. In some embodiments, the target polynucleotide comprises from about 10 to about 80 nucleotides. In some embodiments, the target polynucleotide comprises from about 10 to about 70 nucleotides. In some embodiments, the target polynucleotide comprises from about 10 to about 60 nucleotides. In some embodiments, the target polynucleotide comprises from about 10 to about 50 nucleotides. In some embodiments, the target polynucleotide comprises from about 10 to about 40 nucleotides. In some embodiments, the target polynucleotide comprises from about 10 to about 30 nucleotides. In some embodiments, the target polynucleotide comprises from about 10 to about 20 nucleotides. In some embodiments, the target polynucleotide comprises from about 20 to about 100 nucleotides. In some embodiments, the target polynucleotide comprises from about 30 to about 90 nucleotides. In some embodiments, the target polynucleotide comprises from about 40 to about 80 nucleotides. In some embodiments, the target polynucleotide comprises from about 50 to about 70 nucleotides.

In some embodiments, the double-stranded polynucleotide comprising the single-stranded target polynucleotide and a complementary single-stranded polynucleotide (e.g., the source complex) is at a concentration ranging from about 100 nM to about 1 mM. In some embodiments, the target polynucleotide is at a concentration ranging from 200 nM to about 1 mM. In some embodiments, the target polynucleotide is at a concentration ranging from 400 nM to about 1 mM. In some embodiments, the target polynucleotide is at a concentration ranging from 800 nM to about 1 mM. In some embodiments, the target polynucleotide is at a concentration ranging from 1 μM to about 1 mM. In some embodiments, the target polynucleotide is at a concentration ranging from 100 μM to about 1 mM. In some embodiments, the target polynucleotide is at a concentration ranging from 200 μM to about 1 mM. In some embodiments, the target polynucleotide is at a concentration ranging from 400 μM to about 1 mM. In some embodiments, the target polynucleotide is at a concentration ranging from 600 μM to about 1 mM. In some embodiments, the target polynucleotide is at a concentration ranging from 800 μM to about 1 mM. In some embodiments, the target polynucleotide is at a concentration ranging from 200 nM to about 800 μM. In some embodiments, the target polynucleotide is at a concentration ranging from 400 nM to about 600 μM. In some embodiments, the target polynucleotide is at a concentration ranging from 600 nM to about 400 μM. In some embodiments, the target polynucleotide is at a concentration ranging from 800 nM to about 200 μM. In some embodiments, the target polynucleotide is at a concentration ranging from 1 μM to about 100 μM.

In some embodiments, the target polynucleotide includes at least one toehold domain. In some embodiments, the target polynucleotide includes include at least one toehold domain, and as many as 10 toehold domains. In some embodiments, the target polynucleotide includes from about 2 toehold domains to about 8 toehold domains. In some embodiments, the target polynucleotide includes from about 3 toehold domains to about 6 toehold domains. In some embodiments, the toehold domain includes from about 0 to about 10 nucleotides. In some embodiments, the toehold domain includes from about 2 to about 10 nucleotides. In some embodiments, the toehold domain includes from about 4 to about 10 nucleotides. In some embodiments, the toehold domain includes from about 5 to about 10 nucleotides. In some embodiments, the toehold domain includes from about 1 to about 9 nucleotides. In some embodiments, the toehold domain includes from about 1 to about 8 nucleotides. In some embodiments, the toehold domain includes from about 1 to about 7 nucleotides. In some embodiments, the toehold domain includes from about 1 to about 6 nucleotides. In some embodiments, the toehold domain includes from about 1 to about 5 nucleotides.

Embodiments of the present disclosure include buffered compositions and systems for modulating the concentration of a polynucleotide. In accordance with these embodiments, the compositions and systems can include a single-stranded initiator polynucleotide capable of associating with the source complex to displace the target polynucleotide from the source complex (FIG. 1). In some embodiments, the concentration of the target polynucleotide can be modulated by altering the concentrations of at least one of the source complex or the initiator polynucleotide. The source complex and/or the initiator polynucleotide can each contain a single-stranded target polynucleotide, or part of a single-stranded target polynucleotide.

In some embodiments, the initiator polynucleotide is at a concentration ranging from about 100 nM to about 1 mM. In some embodiments, the initiator polynucleotide is at a concentration ranging from 200 nM to about 1 mM. In some embodiments, the initiator polynucleotide is at a concentration ranging from 400 nM to about 1 mM. In some embodiments, the initiator polynucleotide is at a concentration ranging from 800 nM to about 1 mM. In some embodiments, the initiator polynucleotide is at a concentration ranging from 1 μM to about 1 mM. In some embodiments, the initiator polynucleotide is at a concentration ranging from 100 μM to about 1 mM. In some embodiments, the initiator polynucleotide is at a concentration ranging from 200 μM to about 1 mM. In some embodiments, the initiator polynucleotide is at a concentration ranging from 400 μM to about 1 mM. In some embodiments, the initiator polynucleotide is at a concentration ranging from 600 μM to about 1 mM. In some embodiments, the initiator polynucleotide is at a concentration ranging from 800 μM to about 1 mM. In some embodiments, the initiator polynucleotide is at a concentration ranging from 200 nM to about 800 μM. In some embodiments, the initiator polynucleotide is at a concentration ranging from 400 nM to about 600 μM. In some embodiments, the initiator polynucleotide is at a concentration ranging from 600 nM to about 400 μM. In some embodiments, the initiator polynucleotide is at a concentration ranging from 800 nM to about 200 μM. In some embodiments, the initiator polynucleotide is at a concentration ranging from 1 μM to about 100 μM.

In some embodiments, the initiator polynucleotide comprises from about 10 to about 100 nucleotides. In some embodiments, the initiator polynucleotide comprises from about 10 to about 90 nucleotides. In some embodiments, the initiator polynucleotide comprises from about 10 to about 80 nucleotides. In some embodiments, the initiator polynucleotide comprises from about 10 to about 70 nucleotides. In some embodiments, the initiator polynucleotide comprises from about 10 to about 60 nucleotides. In some embodiments, the initiator polynucleotide comprises from about 10 to about 50 nucleotides. In some embodiments, the initiator polynucleotide comprises from about 10 to about 40 nucleotides. In some embodiments, the initiator polynucleotide comprises from about 10 to about 30 nucleotides. In some embodiments, the initiator polynucleotide comprises from about 10 to about 20 nucleotides. In some embodiments, the initiator polynucleotide comprises from about 20 to about 100 nucleotides. In some embodiments, the initiator polynucleotide comprises from about 30 to about 90 nucleotides. In some embodiments, the initiator polynucleotide comprises from about 40 to about 80 nucleotides. In some embodiments, the initiator polynucleotide comprises from about 50 to about 70 nucleotides.

In some embodiments, the initiator polynucleotide includes at least one toehold domain. In some embodiments, the initiator polynucleotide includes include at least one toehold domain, and as many as 10 toehold domains. In some embodiments, the initiator polynucleotide includes from about 2 toehold domains to about 8 toehold domains. In some embodiments, the initiator polynucleotide includes from about 3 toehold domains to about 6 toehold domains. In some embodiments, the toehold domain includes from about 0 to about 10 nucleotides. In some embodiments, the toehold domain includes from about 2 to about 10 nucleotides. In some embodiments, the toehold domain includes from about 4 to about 10 nucleotides. In some embodiments, the toehold domain includes from about 5 to about 10 nucleotides. In some embodiments, the toehold domain includes from about 1 to about 9 nucleotides. In some embodiments, the toehold domain includes from about 1 to about 8 nucleotides. In some embodiments, the toehold domain includes from about 1 to about 7 nucleotides. In some embodiments, the toehold domain includes from about 1 to about 6 nucleotides. In some embodiments, the toehold domain includes from about 1 to about 5 nucleotides.

In some embodiments, the compositions and systems of the present disclosure include a sink complex, wherein the sink complex comprises a double-stranded polynucleotide comprising the single-stranded initiator polynucleotide and a complementary single-stranded polynucleotide. In accordance with these embodiments, the complementary single-stranded polynucleotide can be at least partially complementary to both the target polynucleotide and the initiator polynucleotide. In some embodiments, the double-stranded polynucleotide comprising the single-stranded initiator polynucleotide and a complementary single-stranded polynucleotide (e.g., the sink complex) is at a concentration ranging from about 100 nM to about 1 mM.

In some embodiments, the concentration of the initiator polynucleotide, the concentration of the double-stranded polynucleotide comprising the single-stranded initiator polynucleotide and a complementary single-stranded polynucleotide, and the concentration of the double-stranded polynucleotide comprising the single-stranded target polynucleotide and a complementary single-stranded polynucleotide are higher than the concentration of the single-stranded target polynucleotide.

In some embodiments, the compositions and systems of the present disclosure include a reporter complex comprising a reporter molecule. In some embodiments, the reporter complex also includes a double-stranded polynucleotide comprising a single-stranded reporter polynucleotide and a complementary single-stranded quencher polynucleotide, wherein the reporter polynucleotide is at least partially complementary to both the quencher polynucleotide and the target polynucleotide. Exemplary reporter molecules include, but are not limited to, a bioluminescent agent, a chemiluminescent agent, a chromogenic agent, a fluorogenic agent, an enzymatic agent and combinations or derivatives thereof.

In some embodiments, the reporter polynucleotide comprises from about 10 to about 100 nucleotides. In some embodiments, the reporter polynucleotide comprises from about 10 to about 90 nucleotides. In some embodiments, the reporter polynucleotide comprises from about 10 to about 80 nucleotides. In some embodiments, the reporter polynucleotide comprises from about 10 to about 70 nucleotides. In some embodiments, the reporter polynucleotide comprises from about 10 to about 60 nucleotides. In some embodiments, the reporter polynucleotide comprises from about 10 to about 50 nucleotides. In some embodiments, the reporter polynucleotide comprises from about 10 to about 40 nucleotides. In some embodiments, the reporter polynucleotide comprises from about 10 to about 30 nucleotides. In some embodiments, the reporter polynucleotide comprises from about 10 to about 20 nucleotides. In some embodiments, the reporter polynucleotide comprises from about 20 to about 100 nucleotides. In some embodiments, the reporter polynucleotide comprises from about 30 to about 90 nucleotides. In some embodiments, the reporter polynucleotide comprises from about 40 to about 80 nucleotides. In some embodiments, the reporter polynucleotide comprises from about 50 to about 70 nucleotides.

In some embodiments, the compositions and systems of the present disclosure include a competitor complex. In some embodiments, the competitor complex also includes a double-stranded polynucleotide comprising a first single-stranded competitor polynucleotide and a second complementary single-stranded competitor polynucleotide, wherein the first competitor polynucleotide is at least partially complementary to both the second competitor polynucleotide and the target polynucleotide. In some embodiments, the competitor polynucleotide comprises from about 10 to about 100 nucleotides. In some embodiments, the competitor polynucleotide comprises from about 10 to about 90 nucleotides. In some embodiments, the competitor polynucleotide comprises from about 10 to about 80 nucleotides. In some embodiments, the competitor polynucleotide comprises from about 10 to about 70 nucleotides. In some embodiments, the competitor polynucleotide comprises from about 10 to about 60 nucleotides. In some embodiments, the competitor polynucleotide comprises from about 10 to about 50 nucleotides. In some embodiments, the competitor polynucleotide comprises from about 10 to about 40 nucleotides. In some embodiments, the competitor polynucleotide comprises from about 10 to about 30 nucleotides. In some embodiments, the competitor polynucleotide comprises from about 10 to about 20 nucleotides. In some embodiments, the competitor polynucleotide comprises from about 20 to about 100 nucleotides. In some embodiments, the competitor polynucleotide comprises from about 30 to about 90 nucleotides. In some embodiments, the competitor polynucleotide comprises from about 40 to about 80 nucleotides. In some embodiments, the competitor polynucleotide comprises from about 50 to about 70 nucleotides.

The buffered oligonucleotide compositions and systems of the present disclosure include various types of oligonucleotide components. In some embodiments, the target polynucleotide and the initiator polynucleotide comprise at least one of a DNA molecule, an RNA molecule, a modified nucleic acid, or a combination thereof. Modified nucleic acids include, but are not limited to, phosphorothioate DNA, peptide nucleic acids (PNA), phosphoramidate DNA, morpholinos, phosphonoacetate (PACE), 2′-O-methoxyethyl RNA, locked nucleic acids (LNA), amide-linked nucleic acids, boranophosphate nucleic acids, and 2′-5′-phosphodiester nucleic acids.

Embodiments of the present disclosure provide methods of modulating concentration of a polynucleotide. In accordance with these embodiments, the methods include formulating a composition that includes a source complex comprising a single-stranded target polynucleotide and a single-stranded initiator polynucleotide capable of associating with the source complex to displace the target polynucleotide from the source complex, and increasing or decreasing the concentration of the initiator polynucleotide in the composition to modulate the concentration of the target polynucleotide.

In some embodiments, the method further includes a sink complex, wherein the sink complex includes a double-stranded polynucleotide comprising the single-stranded initiator polynucleotide and a complementary single-stranded polynucleotide. In some embodiments, the complementary single-stranded polynucleotide is at least partially complementary to both the target polynucleotide and the initiator polynucleotide.

In some embodiments of the method, the source complex includes a double-stranded polynucleotide comprising the single-stranded target polynucleotide and a complementary single-stranded polynucleotide. In some embodiments, the complementary single-stranded polynucleotide is at least partially complementary to both the target polynucleotide and the initiator polynucleotide.

In some embodiments, the method further includes a reporter complex that includes a reporter molecule. In some embodiments, the reporter complex includes a double-stranded polynucleotide comprising a single-stranded reporter polynucleotide and a complementary single-stranded quencher polynucleotide. In some embodiments, the reporter polynucleotide is at least partially complementary to both the quencher polynucleotide and the target polynucleotide.

In some embodiments, the method further includes a competitor complex, wherein the competitor complex comprises a double-stranded polynucleotide comprising a first single-stranded competitor polynucleotide and a second complementary single-stranded competitor polynucleotide. In some embodiments, the first competitor polynucleotide is at least partially complementary to both the second competitor polynucleotide and the target polynucleotide.

In some embodiments, modulation of the target polynucleotide includes increasing the concentration of the target polynucleotide. In some embodiments, the target polynucleotide displaces a small molecule target bound to an aptamer by binding to at least a portion of the aptamer.

Embodiments of the present disclosure also include systems for modulating concentration of two or more polynucleotides. In accordance with these embodiments, the system can include a first composition comprising a first source complex comprising a first single-stranded target polynucleotide and a first single-stranded initiator polynucleotide capable of associating with the first source complex to displace the first target polynucleotide from the first source complex, and at least a second composition comprising a second source complex comprising a second single-stranded target polynucleotide and a second single-stranded initiator polynucleotide capable of associating with the second source complex to displace the second target polynucleotide from the second source complex. In some embodiments, the concentrations of the first and second target polynucleotides are modulated independently within the system by altering the concentrations of at least one of the first and second source complexes or the first and second initiator polynucleotides. In some embodiments, the system can include from 2 to 10 buffered oligonucleotide compositions. In some embodiments, the system can include from 3 to 10 buffered oligonucleotide compositions. In some embodiments, the system can include from 4 to 10 buffered oligonucleotide compositions. In some embodiments, the system can include from 5 to 10 buffered oligonucleotide compositions. In some embodiments, the system can include from 6 to 10 buffered oligonucleotide compositions. In some embodiments, the system can include from 7 to 10 buffered oligonucleotide compositions. In some embodiments, the system can include from 8 to 10 buffered oligonucleotide compositions. In some embodiments, the system can include from 3 to 9 buffered oligonucleotide compositions. In some embodiments, the system can include from 4 to 8 buffered oligonucleotide compositions. In some embodiments, the system can include from 5 to 7 buffered oligonucleotide compositions.

In some embodiments, modulation of the target polynucleotide includes increasing the concentration of the target polynucleotide. In some embodiments, the target polynucleotide alters one or more conformation properties of a nucleic acid-based hydrogel. In some embodiments, nucleic acids can be attached to various hydrogels and solid supports, beads, substrates, and the like, to facilitate strand release. For example, nucleic acid strands can be conjugated to a variety of solid supports including hydrogels, nanoparticles, and microparticles, as well as glass, metal, and polymeric surfaces. Mediation of nucleic acid release through strand displacement (e.g., buffering systems) can be used to buffer nucleic acid strands between states in which the strands are attached to a solid support, or are free from attachment to a solid support. In some embodiments, switching between these two states could be used to determine bioavailability (e.g., controlling drug dosing), for driving chemical processes, and/or for diagnostic purposes.

In some embodiments, the oligonucleotide buffering systems and methods of the present disclosure can incorporate various nucleic acid attachment technology to facilitate the generation and modulation of libraries containing non-DNA molecules (e.g., chemical synthesis). For example, and without limitation, nucleic acid attachment technology can include the use of acrydite, adenylation, azide modifications, digoxigenin, cholesterol-TEG, I-Linker technology, amino modifiers (e.g., Amino Modifiers C6, C12, C6 dT, Uni-Link, and the like from IDT Technologies), 5′hexynyl modifications, 5-octadinynl dU modifications, biotin (e.g., biotin azide, biotin dT, biotin-TEG, dual biotin, PC biotin, and desthiobiotin-TEG), and thiol modifications (e.g., thiol modifier C3 S—S, dithiol, and thiol modifier C6 S—S).

As described further herein, the oligonucleotide buffering systems and methods of the present disclosure can find use in many different contexts. For example, the oligonucleotide buffering systems and methods of the present disclosure can be used for RNA strand displacement in a therapeutic context, such as described in Hochrein, L. M., et al., J. Am. Chem. Soc. 2013, 135, 17322-17330, and Rupaimoole, R. and Slack, F. J., Nature Reviews, Drug Discovery, 2017, 16, 203-221. In some embodiments, the buffering systems and methods of the present disclosure can be applied to RNA strand displacement technologies, such as to control the release of a microRNA and to modulate concentrations of single-stranded RNA molecules in a therapeutic context (e.g., mRNA suppressors such as anti-miRNAs).

In some embodiments, the buffering systems and methods of the present disclosure can include DNA circuits that direct the capture or release of molecules from an aptamer by binding to the aptamer, thus changing the aptamer's binding affinity for its target. In some embodiments, the buffering systems and methods of the present disclosure can be used in pH-dependent processes (e.g., at low pH, the formation of a motif colocalizes a strand to a complex, initiating a strand-displacement reaction that releases an interface strand of DNA). In some embodiments, the buffering systems and methods of the present disclosure can include the use of electronic devices to provide inputs to DNA circuits through electrodes that release or activate interface strands of DNA. DNA strands can produce or regulate voltage or current by regulating the distance between an electrode and an electrically active molecular tag. The close proximity of the electrode to the tag can generate a Faradaic current. In some embodiments, the buffering systems and methods of the present disclosure can be designed to operate at a particular set temperature, rather than to respond to temperature as an input. In some embodiments, DNA hairpin structures can be designed to serve as temperature-responsive thermometers by tuning the strength, and thereby the melting temperature, of the stem domains that hold the hairpins closed. In some embodiments, the buffering systems and methods of the present disclosure can include the use of light to direct the release of a specific DNA sequence by controlling the degradation or conformational change of reagents that initially block or sequester a DNA domain from participating in downstream reactions. In some embodiments, the buffering systems and methods of the present disclosure can be used to transduce a DNA hybridization event into a signal that alters the optical properties of the solution is to use a fluorescent molecule conjugated to the end of a strand of DNA to emit different intensities of light at a target wavelength, depending on the state of the conjugated DNA strand. Emission from the fluorophore can be quenched by a nearby quencher molecule or transferred by FRET (fluorescence resonance energy transfer) to a different fluorophore, which effectively changes the wavelength of the fluorescence output signal. An interface strand of DNA can change the distance between these different fluorophore and quencher modifications by opening a fluorophore-modified hairpin, or displacing a fluorophore-modified strand from a complex. The kinetics of many of the devices described in this review are monitored by measuring changes in fluorescence from fluorescently modified DNA complexes.

### 3. DNA NANOSTRUCTURE ASSEMBLY

Embodiments of the present disclosure include a chemical regulatory mechanism that can sustain growth in a batch crystallization process by resisting changes in chemical potential during the growth process. To study the implementation of such a mechanism, DNA nanotubes were used as a model crystallization system, though as would be recognized by one of ordinary skill in the art based on the present disclosure, the systems and methods provided herein are applicable to the formation, growth, and/or assembly of any DNA-based nanostructure (e.g., nanowires, nanoribbons, nanoarrays, nanopolyhedra, nanocubes, nanoboxes, irregular nanostructures like nanobears and nanovases, and the like).

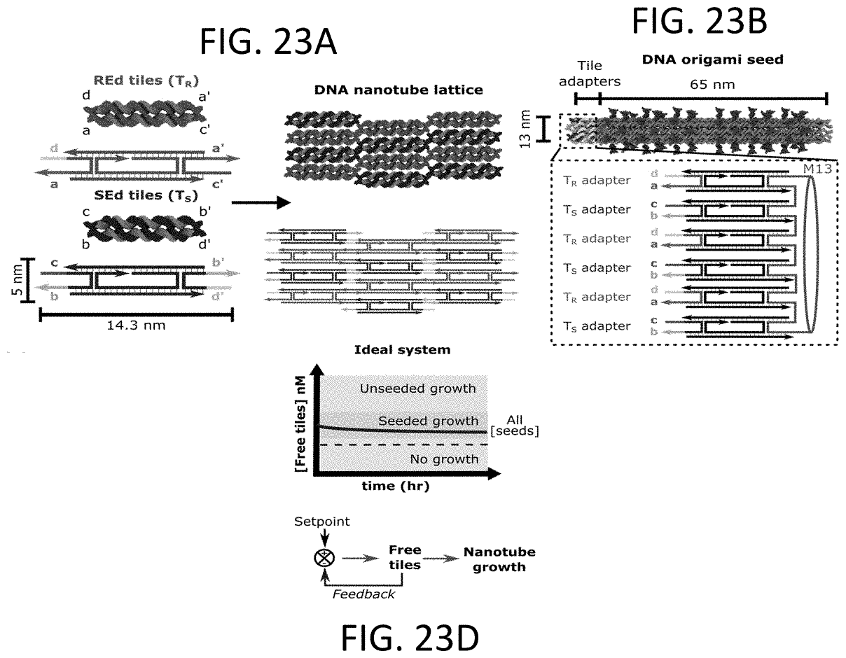

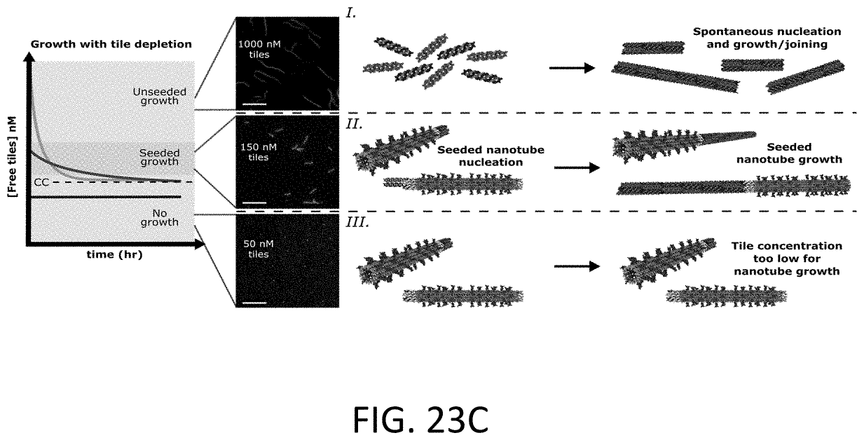

DNA nanotubes are composed of monomers, termed DNA tiles. Each tile is composed of 5 strands of synthetic DNA that fold into a rigid double crossover structure that presents 4 single-strand domains termed sticky ends. Through hybridization of the sticky ends, DNA tiles can self-assemble into lattices that cyclize into nanotubes roughly 10 nM in diameter that can grow to tens of micrometers in length (FIG. 23A). A cylindrical DNA origami seed that mimics the growth face of a DNA nanotube can be used to specifically nucleate nanotubes only from the seed, enabling precise control over when and where growth occurs (FIG. 23B). Since DNA nanotubes are one dimensional crystals, they have a particularly narrow tile concentration range in which only seeded nanotube growth can be achieved (FIG. 23C) making them an ideal system for studying the effects of monomer depletion on crystal growth in batch reactions.

Embodiments of the present disclosure also include a chemical reaction network, using DNA strand displacement reactions that resists changes in the free tile concentration during DNA nanotube growth. The reaction network functions similarly to a pH buffer by shifting its equilibrium to produce more tiles as they are depleted to resist changes in the equilibrium tile concentration. Results provided herein demonstrate that the tile concentration buffering reaction expands the range of seed concentrations over which significant seeded nanotube growth can occur in batch reactions. Further, tile concentration buffering greatly extends the active growth time and is able to sustain growth through temporal changes in growth demand. Additionally, a quantitative kinetic model of the coupled growth and buffering processes was developed, and the model was used to investigate routes to further improved performance. The results provided herein demonstrate how chemical regulation can be implemented to sustain active crystallization over a broader range of growth conditions in batch reactions. The sustained and robust growth demonstrated can enable the self-assembly of more complex hierarchical and dynamic synthetic structures like those seen in the cellular cytoskeleton, for example.

The results provided herein demonstrate a chemical feedback mechanism based on reversible monomer production that resists changes in monomer concentrations during batch crystallization of DNA nanotubes. The tile concentration buffering system is able to sustain batch nanotube growth for extended periods of time, enable growth at high seed concentrations, and adapt to temporal changes in growth demand. These properties facilitate the assembly of a variety of complex DNA nanotube structures or networks with interesting dynamic behavior. For example, hierarchical structures could be built via the sequential addition or activation of different branched growth sites. Further, sustained growth should allow nanotubes to heal themselves after damage; this could be important for applications in harsh environments such as serum. Such nanotube structures could have applications as scaffolds for inorganic or biological materials, conduits for molecular transport.

DNA tile sequences were used to design the reaction network even though the single-stranded Sink strands were predicted to have significant secondary structure; despite this, the systems provided herein were effective. Additionally, the buffering reactions for the two tile types likely have different rates and thus the two tile types probably equilibrate to different equilibrium concentrations in the reactions. Yet there was no need to tune the relative concentrations of the two tile's buffering components to achieve functionality. Differences in the concentrations of the two tile types may not be particularly important for performance because the tile type with the lowest concentration will ultimately control the nanotube growth process. Results provided herein also demonstrate that the Source and Initiator complexes, when present at high concentrations, negatively affected nanotube growth at a fixed tile concentration, yet nanotube growth with buffering was effective. The buffering reactions may compensate for some of the negative interactions between the active tiles and the buffering species.

The simplicity and robustness of the DNA strand displacement buffering systems and methods of the present disclosure facilitate their adaptation to 2D and 3D DNA tile-based crystals. Also, the oligonucleotide buffering systems and methods of the present disclosure can be readily adopted to other DNA-based crystallization processes as well, such as self-assembly of interacting DNA origamis or nanoparticles/colloidals. Beyond nucleic acid structures, a monomer concentration buffering scheme can be implemented for any batch crystallization process. Although DNA strand displacement is described in various embodiments of the present disclosure and is advantageous due to the simplicity of programming the desired reaction network, similar reaction schemes can be readily implemented for self-assembling protein fibers or inorganic molecules.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Materials and Methods.

All DNA strands were obtained from Integrated DNA Technologies (IDT), using the purification options listed below in Table 2. On arrival, all strands were suspended in Millipore purified water at a concentration of about 1 mM and stored at −20° C. Stock concentrations were determined by measuring the absorbance of light at a wavelength of 260 nm (OD260), together with the extinction coefficient for each strand provided by IDT (EXT), using the Beer-Lambert law: [ssDNA]=OD260/EXT.

Each of the double-stranded complexes (Source, Sink, Reporter, and Threshold) were prepared separately at 100 μM in Tris-acetate-EDTA buffer with 12.5 mM Mg++(1×TAE/Mg++). Source and Sink complexes were prepared with a 1.2× excess of top strand (120 μM Signal X for the Source, and 120 μM Initiator for the Sink) to ensure all bottom strands were occupied by a top strand. The Reporter complex was prepared with a 2× excess of top strand (200 μM R_{Q}), which helped reverse biased the reporting reaction to report on higher concentrations of Signal X. They were then annealed in an Eppendorf Mastercycler PCR, first heating the solutions to 90° C., holding the temperature constant for 5 minutes, and then cooling at −0.1° C. per every 6 seconds down to 20° C. Source and Sink complexes were purified by polyacrylamide gel electrophoresis (PAGE). Reporter and Threshold were not gel purified.

For gel purification, 15% polyacrylamide gels were cast by mixing 3.25 mL of 19:140% acrylamide/bis solution (Bio-Rad) with 1.3 mL 10×TAE/Mg++ and 8.45 mL Millipore-purified H2O, and initiated polymerization with 75 μL 10% ammonium persulfate (APS, Sigma Aldrich) and 7.5 μL tetramethylethylenediamine (TEMED, Sigma Aldrich). About 200 μL of dsDNA complex was mixed with 6× loading dye (New England Biolabs, product #B7021S) and loaded into a Scie Plas TV100K cooled vertical electrophoresis chamber. Gels were run at 150V and 4° C. for 3 hours and then cut out the purified bands using UV-shadowing at 254 nm^{SI-R1}. The gel bands were chopped into small pieces, mixed with 300 μL of 1×TAE/Mg++ buffer, and then left on a lab bench overnight to allow the DNA to diffuse out of the gel into the buffer. The next day, the buffer was transferred by pipet to a fresh tube, leaving behind as much of the gel as possible. These fresh tubes were centrifuged for 5 minutes to draw any remaining gel pieces to the bottom of the tube, and then transferred to yet another fresh tube, leaving behind ˜50 μL of gel/solution at the bottom. The concentrations of these purified complexes were then measured with an Eppendorf Biophotometer, using the approximate extinction coefficient EXT=EXT_{top_strand}+EXT_{bottom_strand}−3200N_{AT}−2000N_{GC}, where N_{AT} and N_{GC} are the number of hybridized A-T and G-C pairs in each complex, respectively.^{SI-R3} Purified complexes were stored at 4° C.

Reaction kinetics were measured on quantitative PCR (qPCR) machines (Agilent Stratagene Mx3000 and Mx3005 series) at 25° C. Fluorescence was typically measured every 30 seconds for baseline measurements and for the first 1-2 hours after a reaction was triggered by adding Initiator, to accurately capture the early kinetics of a reaction, and then every 5 minutes for the remainder of the experiment to avoid photobleaching the fluorophore. Reactions were prepared in 96-well plates using 50 L/well volume. Each well contained 1×TAE/Mg++ and 1 μM of 20-mer PolyT strands to help displace reactant species from the pipet tips used to add them to the well. In a typical experiment, Millipore-purified H₂O, TAE/Mg++ and PolyT20 strands were first mixed together. Reporter was then added, and a measurement of the baseline reporter fluorescence was taken to determine what fluorescence corresponded to the state of the system with zero output signal concentration added. Other DNA reactant species were then analyzed, in the amounts specified for each experiment, and tracked the resulting kinetics.

DNA components. All oligonucleotides used in this study were synthesized by Integrated DNA Technologies (IDT). The sequences of the tile concentration buffering components are in provided herein. M13mp18 DNA (7,240 bases) was purchased from Bayou Biolabs (Cat #P-107). The sequences of the adapter strands of the DNA origami seed are provided herein. The staple strands and the labeling strands for the DNA origami seed have been previously described. To minimize background for fluorescence imaging of the DNA nanotubes, Source complexes were not fluorescently labeled and the SEd Initiator complex (Is) was designed to be in a quenched fluorescence state. The reaction between the SEd Initiator complex and the SEd Source complex results in the displacement of a quenching strand so that only active SEd tiles have a high fluorescence signal.

Preparation of tile concentration buffering components and DNA origami seeds. All DNA complexes and structures were annealed in an Eppendorf Mastercycler in 40 mM Tris-Acetate, 1 mM EDTA buffer supplemented with 12.5 mM magnesium acetate (TAEM). REd and SEd Source complexes were annealed separately with all of their strands present at 25 μM. REd and SEd Initiator complexes were annealed separately with all their strands present at 50 μM. Samples were initially held at 90° C. for 5 min and then cooled to 20° C. at −1° C./min. Annealed complexes were stored at 4° C. until use.

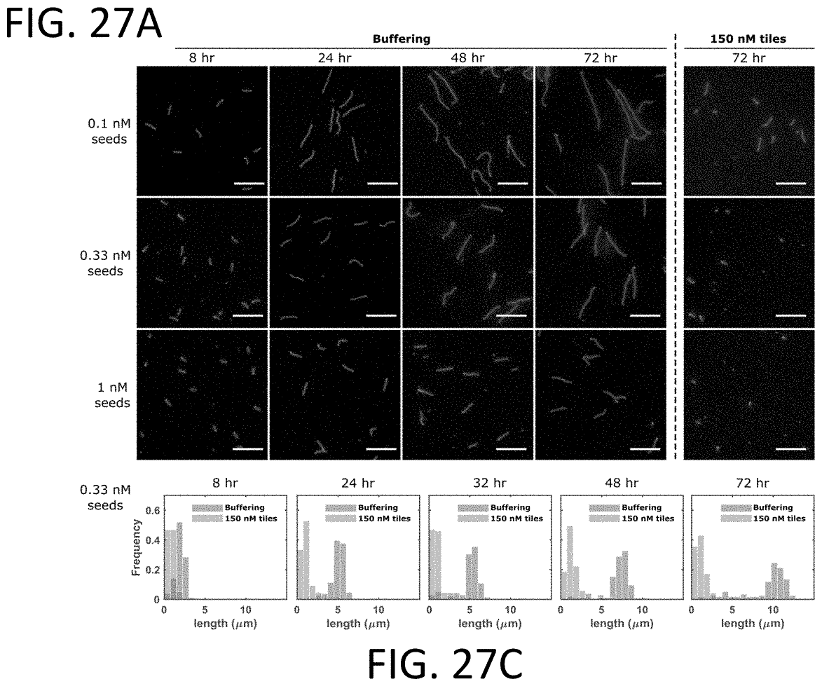

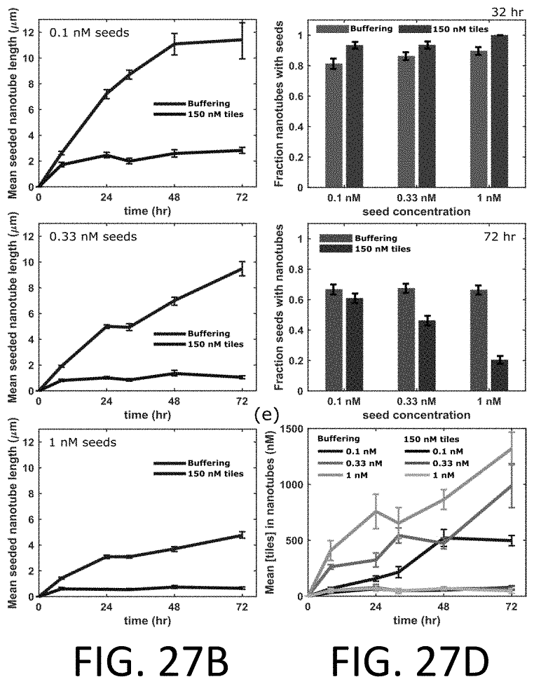

The DNA origami seed was prepared as previously described. The DNA origami seed is composed of a scaffold strand (M13mp18 DNA), 72 staple sequences, and 24 adapter strands. The fluorescence labeling scheme for the seed was as previously described, using a mixture of labeling strands that bind to unfolded M13 DNA and provide a docking site for a fluorescently labeled strand. For all experiments other than those using the S2 seeds, the fluorescently labeled strand used for imaging was labeled with atto488. The S2 seeds were labeled with atto647. The DNA origami seeds were annealed in TAEM buffer with 5 nM M13 DNA, 250 nM of each staple strand, 100 nM of each adapter strand, 10 nM of each labeling strand, and 1000 nM of the fluorescently labeled strand. Biotinylated-BSA 0.05 mg/mL (Cat #A8549, Sigma-Aldrich) was also included to prevent seeds from sticking to the walls of the annealing tubes. Annealing was conducted as follows: samples were incubated at 90° C. for 5 min, cooled from 90° C. to 45° C. at −1° C./min, held at 45° C. for 1 hour, and then cooled from 45° C. to 20° C. at −0.1° C./min. After annealing, seeds were purified with a centrifugal filter (100 kDaA Amico Ultra-0.5 mL, Cat #UFC510096) to remove excess staple, adapter, and labeling strands. For purification, 50 μL of the annealed seed mixture and 250 μL of TAEM buffer was added to the filter and centrifuged at 2000 RCF for 4 min. The samples were washed three more times by adding 200 μL of TAEM buffer in the remain solution and repeating centrifugation, the last wash step was centrifuged at 3000 RCF. The final sample was eluted by inverting the filter in a fresh tube and centrifuging briefly. Seeds labeled with atto647 (FIG. 27) resulted in lower concentrations of purified seeds compared to seeds labeled with atto488. To combat this 100 μL of the annealed seed mixture was used for purification of seeds labeled with atto647. Purified seeds were stored at room temperature until used. Typically, seeds were annealed the day before they were used. Concentrations of purified seeds were determined as previously described.

Nanotube growth experiments. For nanotube growth experiments with a fixed concentration of tiles, the 5 tile strands for both the REd and SEd tiles were mixed at equimolar concentrations in TAEM buffer with 0.05 mg/mL of biotinylated-BSA and 1 μM of a thymine 20-mer. Samples were held at 90° C. for 5 min and then cooled to 20° C. at −1° C./min. Purified seeds were added to the samples during the annealing process when the samples reached 30° C. After annealing the samples were incubated at 20° C. and aliquots were periodically taken for fluorescence imaging.

For nanotube growth experiments with tile concentration buffering, purified seeds, pre-annealed REd and SEd Source complexes (5.5 μM), REd and SEd Sink strands (1.25 μM, unless otherwise stated) were mixed in TAEM buffer with 0.05 mg/mL of biotinylated-BSA and 1 μM of a thymine 20-mer. Pre-annealed REd and SEd Initiator complexes (5.5 μM) were added last to initiate the tile concentration buffering reactions. Samples were incubated at 20° C. and aliquots were periodically taken for fluorescence imaging.

Fluorescence imaging and analysis. Fluorescence imaging was conducted on an inverted microscope (Olympus IX71) using a 60×/1.45 NA oil immersion objective with 1.6× magnification. Images were captured on a cooled CCD camera (iXon3, Andor). For each time point taken for imaging, a small aliquot ( 1/30^{th} of the total reaction volume) was taken and diluted in TAEM with an additional 10 mM magnesium acetate for imaging (additional magnesium acetate facilitated nanotube binding to the glass coverslip). Samples with 0.1 nM seeds were typically diluted 100× for imaging, samples with 0.33 nM seeds were diluted 300×, and samples with 1 nM seeds were diluted 800×. After dilution, 5 μL of each sample was added to an 18 mm by 18 mm glass coverslip (Cat #48366 045, VWR) that was then inverted onto a glass slide (Cat #16004-424, VWR). Images were then captured at 5 to 6 randomly selected locations for each sample. Images were processed and analyzed using custom MATLAB scripts.

Image analysis. The fluorescence micrographs and nanotubes and/or seeds were processed using custom MATLAB scripts for quantitative analysis. Five to six images were typically processed and analyzed for a specific sample at a given timepoint. Below is the workflow for the image analysis process.

Detecting objects. A fluorescence micrograph of DNA nanotubes and a corresponding fluorescence micrograph of DNA origami seeds were imported simultaneously for analysis. Canny edge detection was used to detect the edges of objects in both the DNA nanotube image and the DNA origami seed image and produce binary images of the object edges. The detected objects were then filled in with pixels using MATLAB's bwmorph( ) function. No further processing was done to the DNA origami seed image.

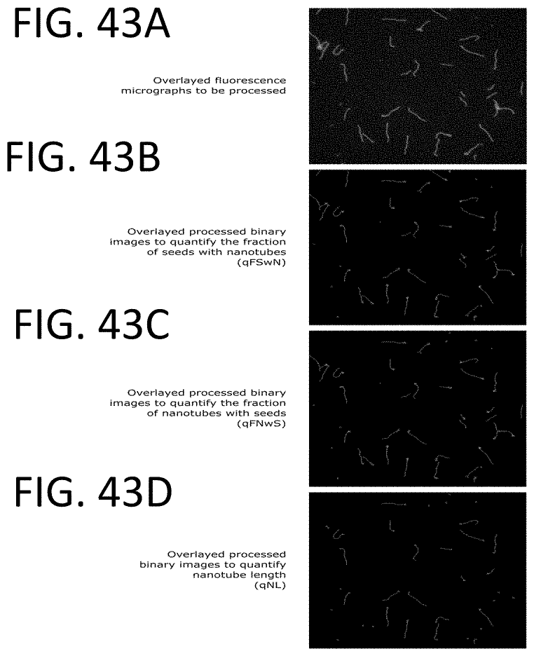

For the DNA nanotube image, morphological operations were applied with MATLAB's bwmorph( ) function to skeletonize all the detected objects to be 1 pixel in width. This processed image was used for the quantification of the fraction of seeds with nanotubes (referred to as qFSwN herein; FIG. 43).

Quantification of fraction of seeds with nanotubes. To quantify the fraction of seeds with nanotubes, the locations of the endpoints of each object in the qFSwN image were determined. A radius (typically 2 to 4 pixels) around each of these endpoint locations was searched in the processed DNA origami seed image and if a seed was found in the search radius this seed was counted as having a nanotube attached to it (FIG. 43B). The fraction of seeds with nanotubes was then calculated as the total number of seeds that had a nanotube attached to them over the total number of seeds across all the images processed for a given sample at a specific timepoint. The total seeds in each image were quantified by counting all the individual objects in the processed DNA origami seed image. Error bars for the fraction of seeds with nanotubes represent the 95% confidence intervals of proportions (CI=±1.96 \sqrt{p(1−p)/n}).

Quantification of fraction of nanotubes with seeds. To quantify the fraction of nanotubes with seeds, the objects in the qFSwN nanotube image that extended past the boundary of the image were removed. Since both ends of nanotubes that extend past the image boundary cannot be analyzed, it is difficult to determine accurately if these nanotubes have a seed attached to them; therefore, these nanotubes were removed for this analysis. This processed nanotube image was termed qFNwS. The locations of the endpoints of each object in the qFNwS image were determined. A radius (typically 2 to 4 pixels) around each of these endpoint locations was searched in the processed DNA origami seed image and if a seed was found in the search radius a nanotube with a seed was counted (FIG. 43C). The fraction of nanotubes was then calculated as the total number of nanotube endpoints that had a seed attached to them over the total number of nanotubes across all the images processed for a given sample at a specific timepoint. Since some nanotubes cross over in the images and result in branched objects with more than two endpoints in the processed images, the total number of nanotubes in an image was calculated as:

$$ceil ( \frac{\# of endpoints}{2} ) \qquad ( 9 )$$

Where ceil(x) rounds to the lowest integer greater than or equal to x. So, an object with two endpoints would be counted as a single nanotube, an object with three or four endpoints would be counted as two nanotubes, an object with five or six endpoints would be counted as three nanotubes, etc. Error bars for the fraction of seeds with nanotubes represent the 95% confidence intervals of proportions.

Quantification of nanotube length. To quantify the length of the nanotubes, the qFNwS image was used since the length of nanotubes that extend beyond the boundary of the image cannot be determined. Additionally, the branched structures in the qFNwS images were removed since the length of nanotubes that cross over cannot be accurately determined. This processed image was termed qNL herein (FIG. 43D). Since each nanotube in the qNL image is represented as a single pixel width object, the length of each nanotube was calculated based on the number of pixels in the nanotube. Based on the resolution and magnification of the camera, each pixel is 170 nm by 170 nm. Each pixel horizontally or vertically connected to another pixel was thus considered as 170 nm of length. Each pixel diagonally connected to another pixel was considered as V Z*170 nm. The mean length of the nanotubes for a specific sample at a given timepoint was calculated from all the nanotube lengths obtained across all the images processed for that sample and timepoint. For samples with seeds only the nanotubes attached to seeds were considered in the length calculation. Error bars for nanotube lengths represent 95% confidence intervals computed using MATLAB's bootstrapci( ) function.

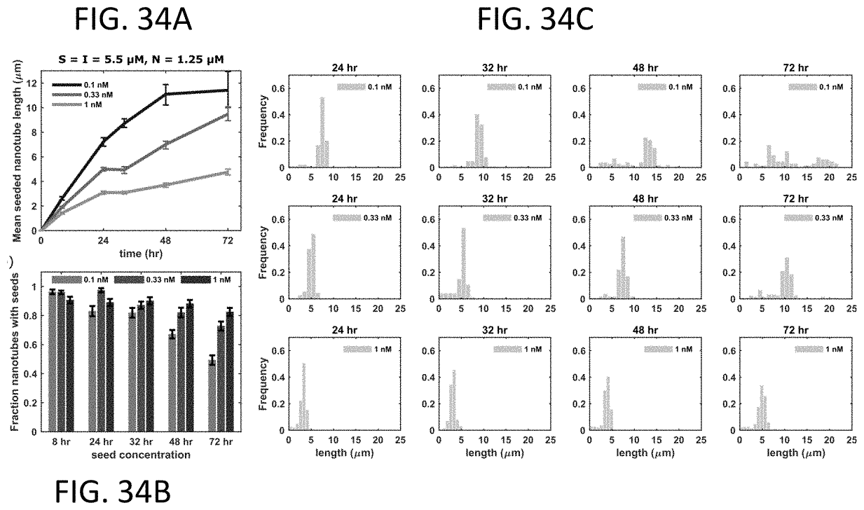

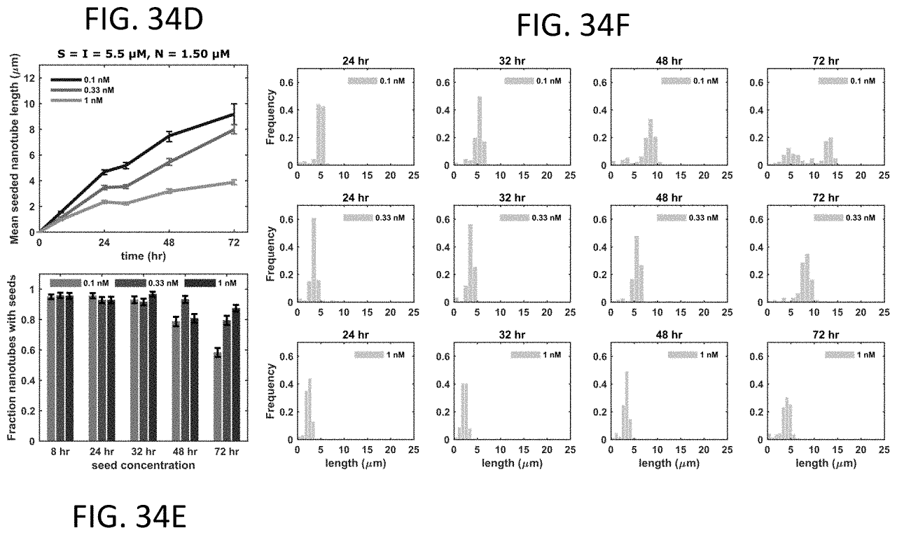

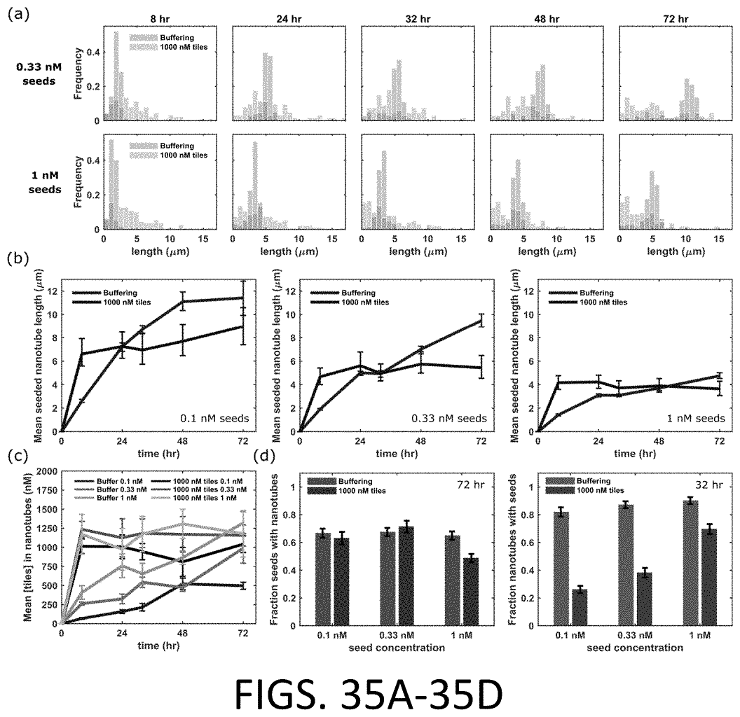

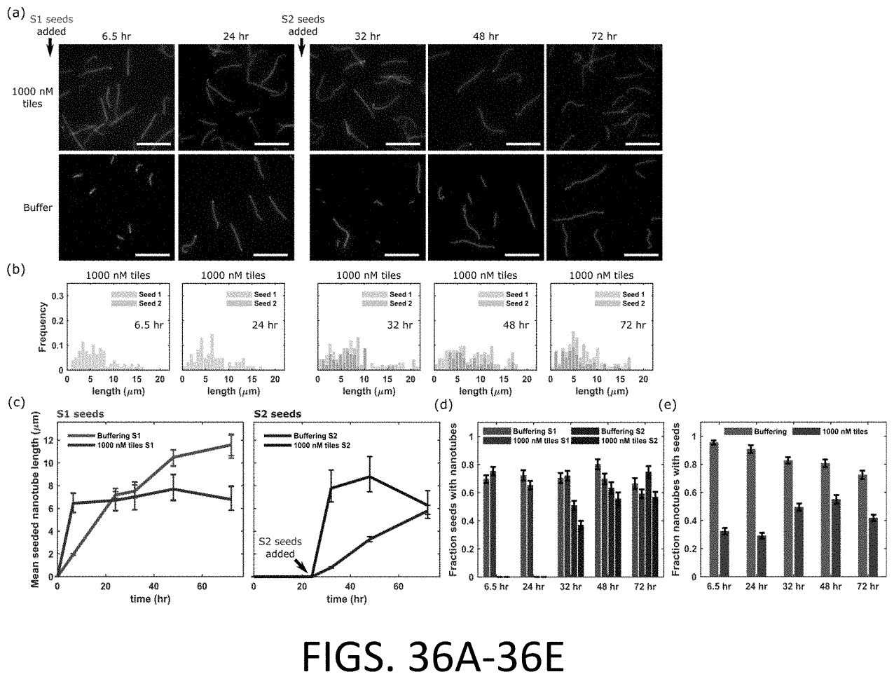

The mean nanotube length for samples with nanotubes longer than 8-10 μm may not be representative of the actual lengths of the nanotubes in the samples as it was observed that long nanotubes were prone to breaking when attaching to the coverslip surface during imaging. Thus, for the tile concentration buffering samples with 0.1 nM seeds (where nanotubes were ˜10 μm after 32 hours) the average lengths determined at the 48 and 72 hours timepoints are skewed towards shorter lengths due to nanotubes breaking (FIG. 34).

Quantification of mean concentration of tiles incorporated into nanotubes. To quantify the mean concentration of tiles incorporated into nanotubes, the total number of nanotube pixels in a qFSwN image was determined and converted to concentration as follows:

$$[ tiles ] in tubes = \frac{( 2 * total pixels * tiles per pixel )}{\begin{matrix}volume on slide * \frac{field of view area}{coverslip area} * \\ {Avogardro}^{′} s \#\end{matrix}} * dilution factor \qquad ( 10 )$$

It was assumed that nanotubes bind to the surface of the coverslip and the glass slide equally so the total pixels determined from the image of the coverslip was doubled (2*total pixels in Eq. 10). Since each pixel is 170 nm, each tile is 14.3 nm, and there are 6 tiles per each row of a nanotube, each pixel was considered to have 72 tiles (tiles per pixel in Eq. 10). Each slide was prepared with 5 μL of the sample (volume on slide). The field of view of each image was 87 μm² and the coverslips used were 18 mm². Since the samples were diluted before imaging, the final concentration obtained was multiplied by the dilution factor used for imaging. Plugging all these values into Eq. 10 yields:

$$[ tiles ] in tubes = \frac{( 2 * total pixels * 72 )}{\begin{matrix}5 * {10}^{- 6} L * {( \frac{0.087 mm}{18 mm} )}^{2} * 6.023 * \\ {10}^{23} \frac{molecules}{mol}\end{matrix}} * dilution factor$$

Error bars for the mean concentration of tiles incorporated into nanotubes represent the standard deviation from image to image.

To validate this quantification approach, the mean concentration of tiles in nanotubes was also calculated based on the mean seeded nanotube lengths and the fraction of seeds with nanotubes as in Eq. 11

$$[ tiles ] in tubes = mean length ( µm ) * \frac{tiles}{µm} * seed concentration * \frac{seeds with nanotubes}{total seeds} \qquad ( 11 )$$

So for the 1 nM seeds sample with tile concentration buffering (FIG. 27):

$$[ tiles ] in tubes = 4.76 µm * \frac{1 tile}{0.0 \; 143 µm} * 6 \frac{tiles}{row} * 1 nM * 0.66 = 1318 nM$$

Table 1 shows the results of the analysis using Eq. 10 compared Eq. 11 across all the samples analyzed in FIG. 27. The two methods produce similar results and the analysis in Eq. 10 was used throughout the present disclosure.

Table 1. Mean concentration of tiles incorporated into nanotubes for the samples presented in FIG. 27. Quantification was conducted as described for Eq. 10 or Eq. 11 above. Ranges for the Eq. 10 values represent standard deviation from image to image from the analysis.

| Tile concentration | | | |
|---|---|---|---|
| buffering | | Eq. 10 analysis | Eq. 11 analysis |
| 0.1 | nM seeds | 497.2 ± 46.1 | 307 nM |
| | | nM | |
| 0.33 | nM seeds | 988.9 ± 197.1 | 866 nM |
| | | nM | |
| 1 | nM seeds | 1320 ± 147.2 | 1318 nM |
| | | nM | |

150 nM tiles

| | | | |
|---|---|---|---|
| 0.1 | nM seeds | 79.1 ± 14.3 | 72 nM |
| | | nM | |
| 0.33 | nM seeds | 71.3 ± 17.9 | 67 nM |
| | | nM | |
| 1 | nM seeds | 47.2 ± 22.2 | 55 nM |
| | | nM | |

### Example 2

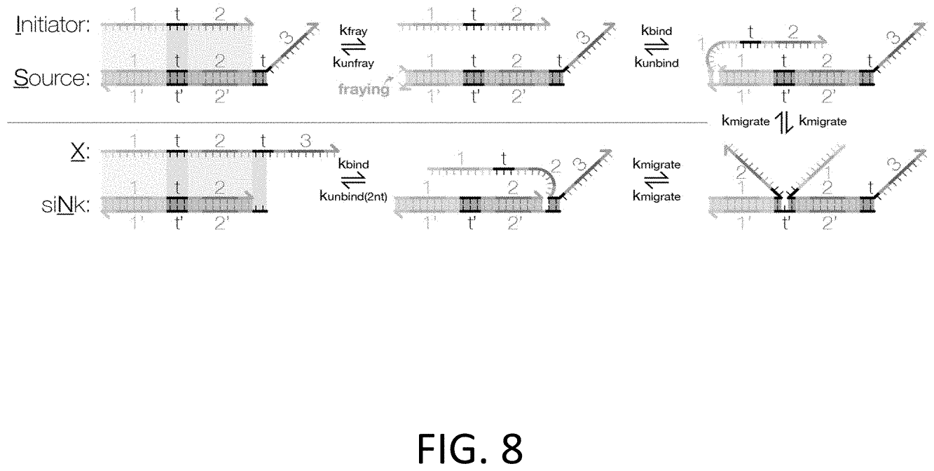

Synthetic buffers for other molecules. The general mechanism underlying pH buffers is not specific to acids and bases. A consequence of Le Chatelier's Principle is that any reversible reaction can serve as a buffer, given appropriate reaction rate constants and provided that all reactants are at a high concentration relative to the regulated species (FIG. 8). This idea was used to regulate the concentration of an arbitrary oligonucleotide in its single-stranded form.

To design a bimolecular buffer that regulates a target species X, a source complex (S) that is a precursor of X was used as a starting point. This source reacts reversibly with an initiator molecule (I), releasing X in an active state, along with a conjugate sink molecule (N) that can recapture X (Eqn 3 below). The species S, I, N & X act as analogs to HA, H₂O, A⁻ & H₃O⁺, respectively, in an acid-base buffer (FIG. 1; Eqn. 1 below).

$$S + I {{\vec{↽}}^{{k}_{S }}}_{ {k}_{N }} N + X \qquad [ 3 ]$$

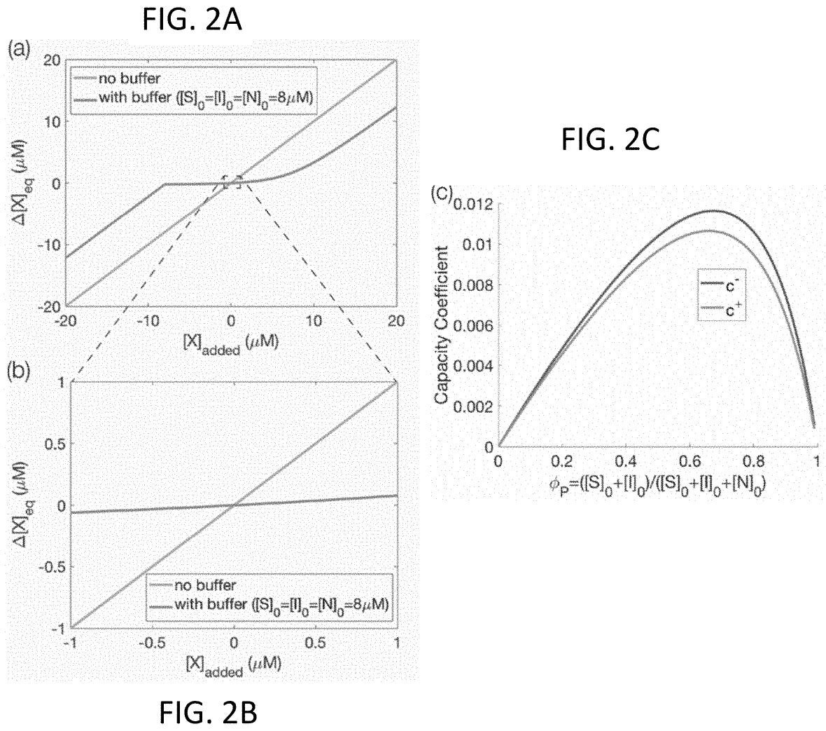

At high reactant concentrations, this reaction creates a stable equilibrium that resists disturbances to X (FIGS. 2A-2B). In this generalized form, the initiator is not necessarily H₂O; therefore, it may be difficult to provide initiator at a concentration such that its depletion can be ignored, as in Eqn. 2 (below). Thus, to describe the operation of this generalized molecular buffer, a more general chemical equilibrium constant is used in place of the dissociation constant Kₐ:

$${K}_{e q} ≡ \frac{{k}_{S}}{{k}_{N}} = \frac{{{[ X ]}_{e q} [ N ]}_{e q}}{{{[ S ]}_{e q} [ I ]}_{e q}} . \qquad [ 4 ]$$

A buffer of the form in Eqn. 3 has three important metrics that describe its performance: the setpoint concentration, the relaxation time constant, and the buffering capacity. Disclosed herein are equations that give order-of-magnitude estimates for how these values scale with the reactant concentrations and rate constants (Appendix A).

The setpoint [X]ₛₑₜ is the equilibrium concentration of X generated by the buffer when [X]₀=0. For high concentrations of [S]₀, [I]₀, and [N]₀, and low equilibrium constants K_{eq}, the setpoint can be approximated as:

$${[ X ]}_{s e t} \approx {K}_{e q} \frac{{{[ S ]}_{o} [ I ]}_{o}}{{[ N ]}_{o}} . \qquad [ 5 ]$$

Eqn. 5 (above) indicates two separate types of parameters for specifying [X]ₛₑₜ: the equilibrium constant K_{eq} and the ratio of the reactant concentrations. Generally, changing K_{eq} requires redesigning the reactant molecules, rather than adjusting their initial concentrations. For simplicity, [S]₀=[I]₀ is applied throughout this disclosure.

The relaxation time constant T determines how quickly X relaxes back towards its setpoint after it is disturbed. The relaxation time constant can be approximated by Eqn. 6 (below), where the time it takes for a disturbance to relax to a defined percentage 0<α<1 of its initial amplitude is given by Eqn. 7 (below).

$$\begin{matrix}T = \frac{1}{{{k}_{N} [ N ]}_{0}} & [ 6 ] \\ {t}_{r e l a x , α} = - τ \cdot ln ( α ) & [ 7 ]\end{matrix}$$

The buffering capacity β determines how much of an external perturbation the buffer can accommodate while maintaining a final equilibrium concentration [X]_{eq} close to the initial setpoint concentration [X]ₛₑₜ. Specifically, β⁺ is the maximum concentration of X that can be added to a buffer while keeping [X]_{eq} below a specified factor of [X]ₛₑₜ (a factor of 1.1 was used here):

[X]_{eq}≤1.1·[X]ₛₑₜ, [8]

and β⁻ is the maximum concentration of X that can be removed from the buffer while keeping [X]_{eq} above an arbitrary factor of [X]ₛₑₜ (a factor of 0.9 is used here):

[X]_{eq}≥0.9·[X]ₛₑₜ. [9]

The resulting capacities are:

β⁺=c⁺·([S]₀+[I]₀+[N]₀) [10]

β⁻=c⁻·([S]₀+[I]₀+[N]₀) [11]

where c⁺ and c⁻ are coefficients determined by the relative ratios of S, I, and N (FIG. 2C). From Eqn's 10-11 (above) it was found that providing the reactants at high concentrations maximizes the capacity to recover from disturbances.

### Example 3

Designing buffers using DNA strand-displacement. Next, DNA strand-displacement (DSD) reactions were used to implement a buffer for DNA oligonucleotides. In DSD reactions, an input strand of DNA binds to a multi-stranded DNA complex, and in the process displaces one or more output strands from the complex. Short single-stranded domains, called toeholds, initiate these reactions and determine the forward and reverse rate constants.

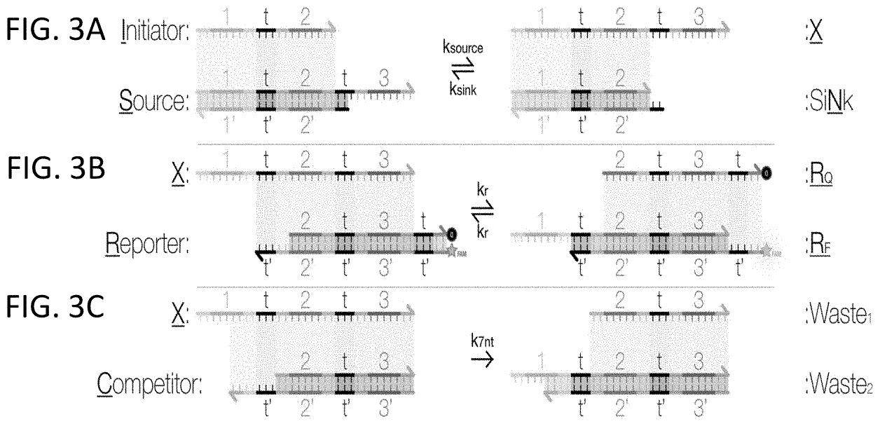

In the DSD implementation (FIG. 3A) of the buffer reaction described in Eqn 3 (above), a target DNA strand was designating as X. Initially, X is bound within a source complex S, such that its toeholds are covered in an inert double-stranded state, preventing downstream reactions. An initiator strand I reversibly displaces X from S, and exposes the toeholds on X. In the process of displacing X, a new sink complex is created that consists of the initiator strand bound to the bottom strand of the source.

Selecting toehold lengths. The forward and reverse reaction rate constants for the DSD buffering reactions are determined by the toehold binding energy for the source and sink complexes, which is correlated with their toehold lengths. Toehold lengths were selected that would create a buffer that holds X at setpoint concentrations [X]ₛₑₜ on the order of 10-100 nM, with relaxation times on the order of 0.5 hours to relax to 10% of any disturbance (t_{relax,α}≈0.5 hr with α=0.1). To maximize the capacity of the buffer (Eqn's 10-11 above), large concentrations of S, I and N were selected, relative to [X]ₛₑₜ, up to 8 μM.

Eqn's 6-7 (above) were used to find a target reverse rate constant k_{N}. Desired relaxation time was input into Eqn 7 to find a time constant of about =0.2 hr. Eqn 6 indicates that to achieve this time constant with [N]₀=8 μM, a sink rate constant on order k_{N}≈2·10⁻⁴ μM⁻¹s⁻¹ can be selected. Similarly, to find a forward rate constant that would result in a setpoint concentration of the desired order with [S]₀=[I]₀=[N]₀=8 μM, Eqn 5 (above) was used to determine an equilibrium constant of roughly K_{eq}≈0.01. Inputting these target values for K_{eq} and k_{N} into Eqn 4 (above), a source rate constant on order k_{S}≈2˜10⁻⁶ μM⁻¹s⁻¹ was found.

Toehold lengths whose average rate constants were closest to desired values were next tested. These lengths turned out to be a zero nucleotide (nt) toehold to drive the forward reaction, which can initiate through fraying at the end of a double stranded complex, and a 2 nt toehold to drive the reverse reaction. (FIG. 3).

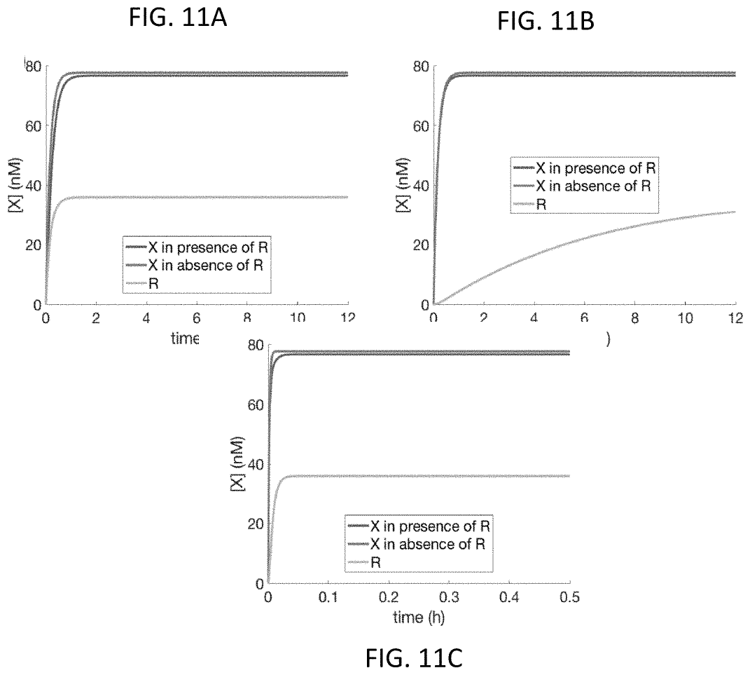

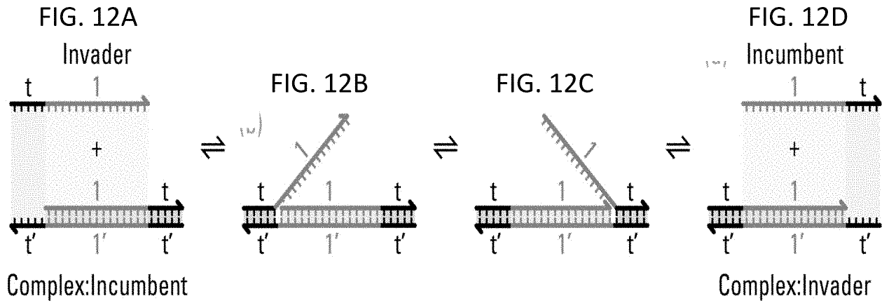

Measuring signal concentrations with a reversible reporting reaction. To monitor the free concentration of X during buffering, a DNA strand displacement reporter with quencher and fluorophore labels were used (FIG. 3B). X reacts reversibly with the reporter, so that when the reaction between X and the reporter is at equilibrium, the magnitude of fluorescence intensity can be used to determine [X] (FIG. 11). To measure accurately the kinetics of the buffering process, a reporter needs to equilibrate significantly faster than the buffer, such that the reporter remains at pseudo-steady-state as [X] changes. To ensure this condition is met, a reporter toehold length of 5 nt was selected to characterize the buffer in FIG. 3A (see also FIG. 12).

### Example 4

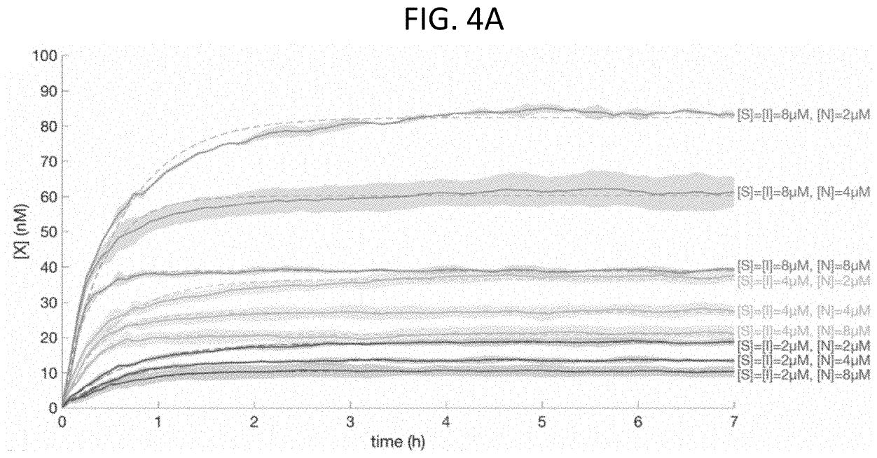

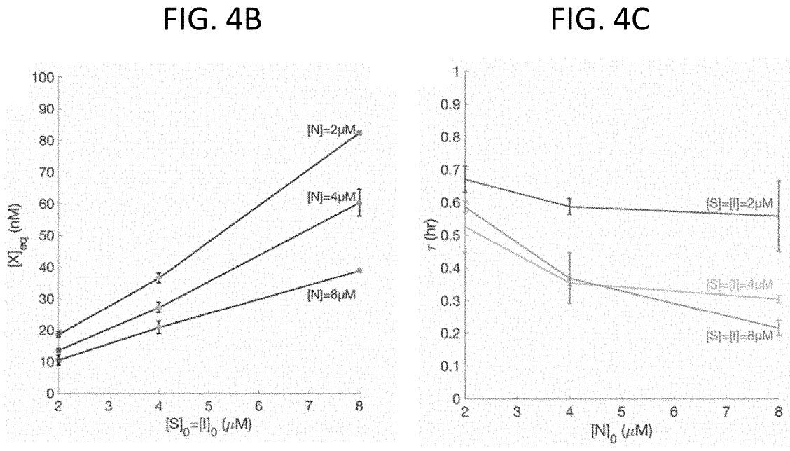

Control over setpoint concentration and relaxation time. To test the prediction (Eqn 6) that the equilibration concentration of X is controlled by the initial concentrations of source (S), initiator (I), and sink (N), these reactants were combined at different initial concentrations, keeping [S]₀=[I]₀. How [X] converged to a stable final value, [X]ₛₑₜ, was measured for each set of initial reactant concentrations (FIG. 4A). For the values of [S]₀, [I]₀, and [N]₀ tested, [X]ₛₑₜ ranged from 7 nM to 83 nM, on the order of the 10-100 nM range of setpoint concentrations. As shown in FIG. 4B, the average equilibrium constant from all curves came to K_{eq}=0.026±0.016, on the same order as the designed value. Following the predictions, it was observed that buffers with higher values of [S]₀=[I]₀ produced higher [X]ₛₑₜ values, and higher values of [N]₀ produced lower [X]ₛₑₜ values (FIG. 4B), also consistent with the design.

The relaxation of the buffer was characterized by fitting the data (FIG. 4C) to

$$\begin{matrix}[ X ] ( t ) = {[ X ]}_{set} ( 1 + ɛ {e}^{- \frac{t}{τ}} ) & [ 12 ] \\ ɛ ≡ \frac{{[ X ]}_{0} - {[ X ]}_{s e t}}{{[ X ]}_{s e t}} , & [ 13 ]\end{matrix}$$

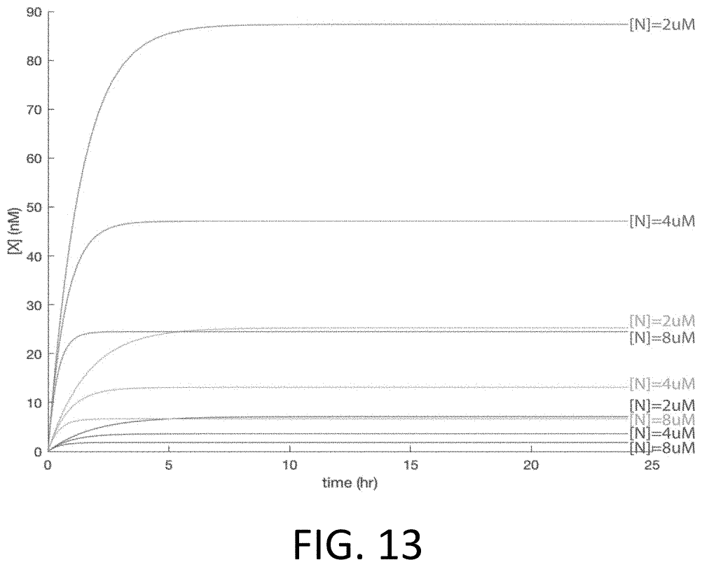

which is the curve predicted by a simple bimolecular mass action model of Eqn 3 (above). For the range of initial concentrations tested, i ranged from about 0.2 to 0.7 hours. Both the simple bimolecular mass action model of the buffer and a more detailed model (FIG. 13) of the buffering and reporting strand-displacement reactions predict that the rise time should be faster as [N]₀ increases, because [N]₀ controls the recapture rate. Consistent with these predictions, it was observed that rise times were faster for higher values of [N]₀. The simple bimolecular model predicts that rise time should be independent of [S]₀=[I]₀, however, the detailed model suggests a slight increase in rise time with decreasing [S]₀=[I]₀ when the reporting reaction is included (FIG. 13). This suggests that the reporter imposes a small load on the system, causing a delay before equilibrium is reached. In these experiments, small increases in rise time for decreasing [S]⁰=[I]₀ were observed.

As shown in FIG. 4C, the average sink reaction rate constant was k_{N}≈1.7·10⁻⁴±4.2·10⁻⁵ μM⁻¹s⁻¹. This constant and the measured K_{eq}=0.026±0.016 gave an average source reaction rate constant of k_{N}≈4.0·10⁻⁶±7.5·10⁻⁷ μM⁻¹s⁻¹. These values are within an order of magnitude of their designed values.

### Example 5

Response to disturbances. Characterization of the relaxation of oligonucleotide buffers as they are perturbed from equilibrium was investigated. The [S]₀=[I]₀=[N]₀=8 μM was used, which was referred to as the uniform 8 μM buffer.

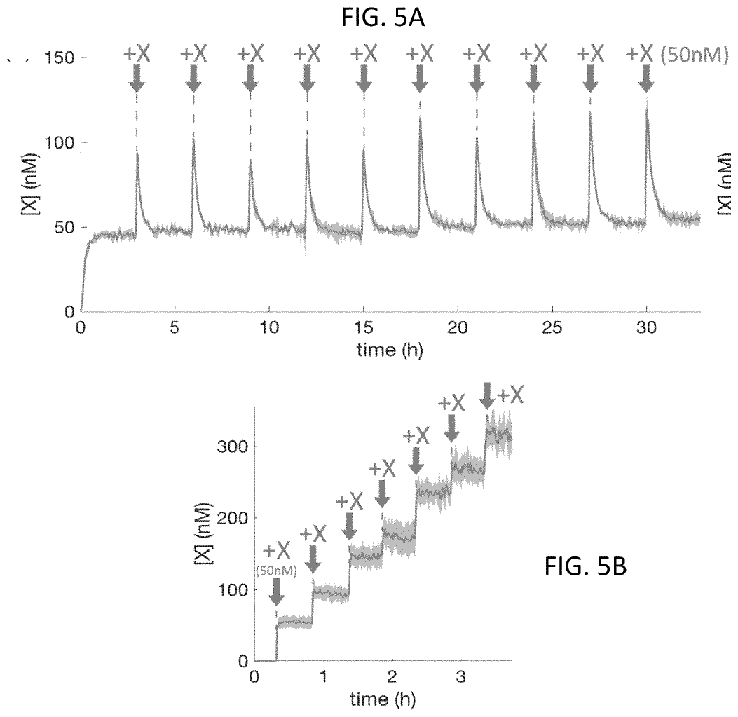

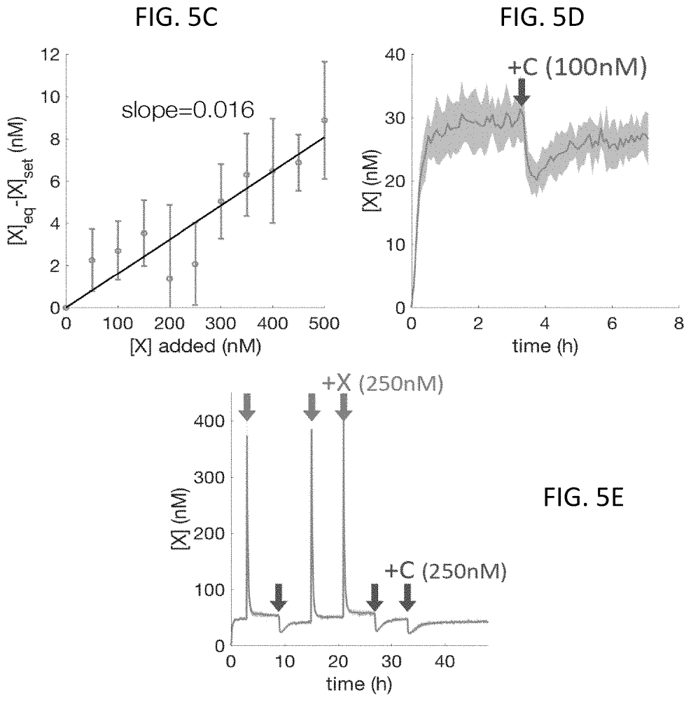

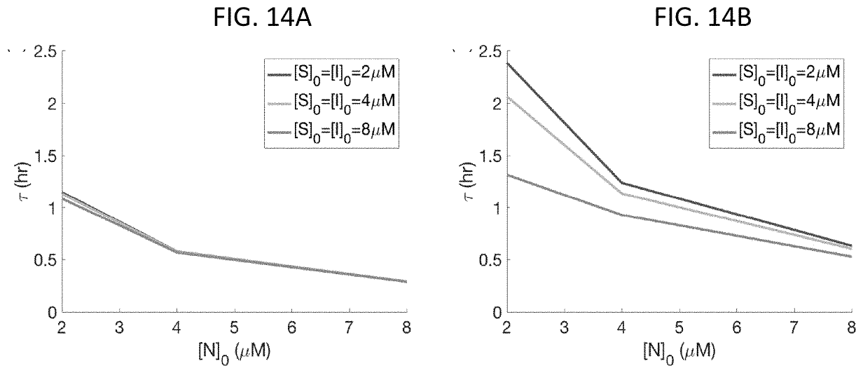

Positive disturbances. First, how oligonucleotide buffers resist positive perturbations (e.g., sudden increases in [X]) was characterized. The uniform 8 μM buffer was allowed to equilibrate, and then a pulse disturbance of 50 nM of X every three hours was added, for a total of ten pulses (FIG. 5A). After each pulse was added, the concentration of X increased quickly, reflecting the X that was added, and then relaxed back to a new equilibrium state close to [X]ₛₑₜ. Eqn 8 (above) was fit to each relaxation and found no significant variation in the time constant r compared to the initial relaxation from [X]₀=0 (FIG. 14). These experiments demonstrate that over at least ten perturbations of this size, the dynamics of relaxation are largely independent of the history of past perturbations. [X]_{eq} increased roughly linearly with the total amount of X added. After 500 nM of X was added, [X]_{eq} increased by 8 nM, which gives a slope of approximately 0.016 (FIG. 5B compared to FIG. 2B). This corresponds well with Eqn 10 (above), which predicts that approximately 250 nM of X must be added to increase the equilibrium concentration by 10%.

Negative disturbances. How the buffer responded to decreases in [X] caused by a coupled reaction in which X was consumed was then characterized. A irreversible reaction in which X binds to a competitor complex via a 7 nt toehold initiated displacement reaction was used, producing inert waste products (FIG. 3C). The 8 μM uniform buffer was allowed to reach steady state for 3 hours, then 100 nM of competitor was added (FIG. 5C). As anticipated, the concentration of X initially dropped, and then recovered to approximately 3 nM less than the initial equilibrium concentration.

The observed magnitude of this negative disturbance was significantly smaller than the concentration of competitor that was added. The models described herein (see also Appendix A) indicate that this effect is due to a partial reaction known as toehold occlusion in which the toehold on the competitor is transiently occupied by the initiator. This interaction reduces the fraction of competitor that is free to react with X, in turn reducing their effective reaction rate. Toehold occlusion is especially significant here due to the long 7 nt toehold and because [I]>>[X]ₛₑₜ, both of which increase the residency time of toehold binding.

Large mixed disturbances. How oligonucleotide buffers respond to very large perturbations were also tested. A uniform 8 μM buffer was allowed to approach its steady state for 3 hours and then it was perturbed first by adding 250 nM of X, then adding 250 nM of the competitor (FIG. 5D). Two more 250 nM pulses of X were then added, followed by two more 250 nM pulses of competitor (FIG. 5D). The changes in [X]_{eq} in the buffered solution were far less in each case than the amount of X that was added or consumed. After the first 250 nM perturbation, [X]_{eq} increased from about 47 to about 49 nM, consistent with the change in [X]_{eq} that occurred after adding five 50 nM pulses of X (FIG. 5B).

### Example 6

Buffering multiple species. Multiple different oligonucleotide sequences can be buffered in the same reaction (see below). A buffering system was designed for a second target sequence, X₂, and how X and X₂ buffers acted separately vs. together in the same reaction was investigated.

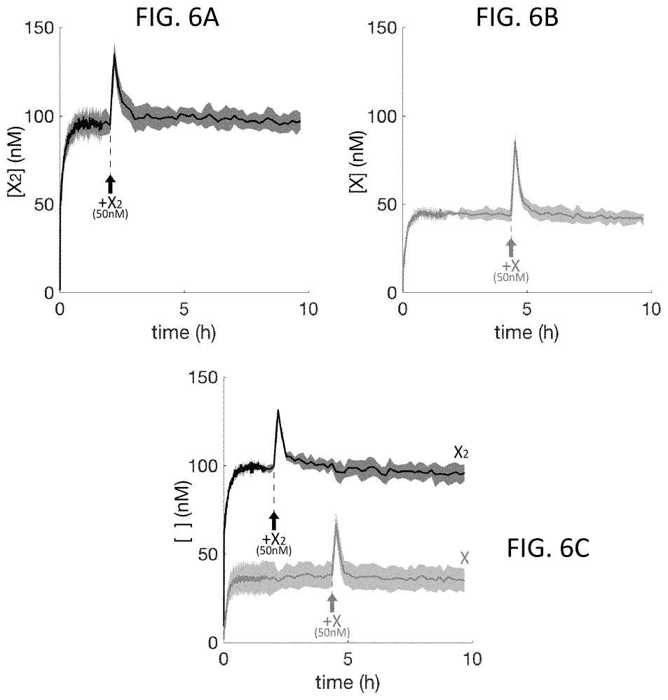

The 8 μM uniform buffer for X₂ reached a stable setpoint of around 100 nM, and after a 50 nM perturbation [X₂] returned to roughly the original setpoint concentration (FIG. 6A). The corresponding 8 μM uniform buffer for X also reached a setpoint and responded to a 50 nM disturbance with a similar response time (FIG. 6B). When the buffers were combined in the same solution, the setpoints and responses to disturbance for both X and X₂ were similar to their setpoints and responses in isolation (FIG. 6C), thus demonstrating functional independence. [X] did not change when X₂ was added and vice versa.

### Example 7

Fast buffering. The buffers in FIGS. 3-5 have relatively slow relaxation times. A slow buffer can allow a system to maintain a memory of recent perturbations that is gradually erased as the concentration of the buffered species returns to equilibrium. This ability to transiently store information about perturbations and then erase it to receive new perturbations could be used to process streams of chemical inputs. However, in many other cases, it is important to maintain a constant concentration in the face of heavy loads. Faster time constants are desirable in such cases. Therefore, whether buffers could be produced that responded more quickly to perturbations was investigated.

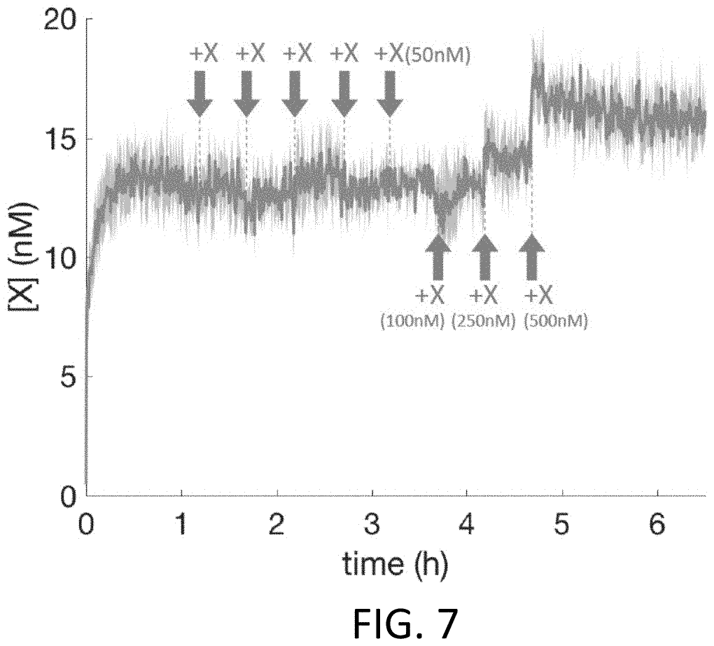

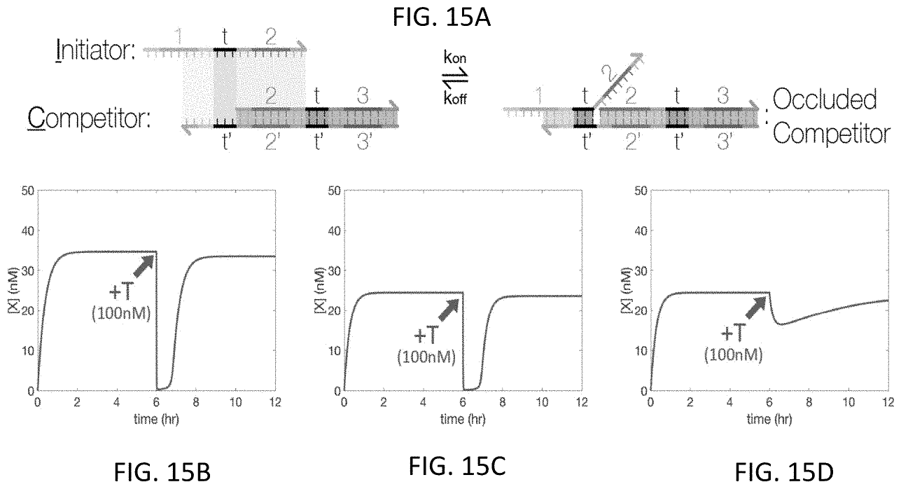

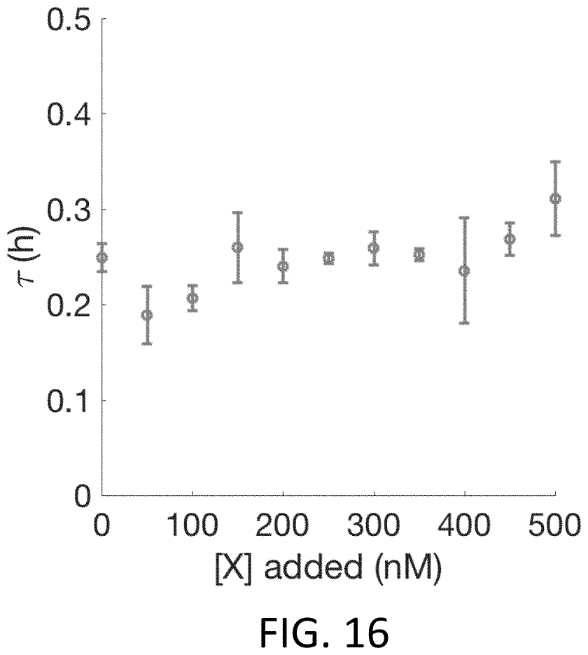

A buffer for X with longer toeholds on the source and sink complexes (+1 and +2 nucleotides respectively) were created and were designed to increase k_{S} and k_{N} (FIG. 11). [X] was characterized over time using 8 μM of each of the new faster source, initiator and sink species (the fast 8 μM uniform buffer; FIG. 7), which produced a setpoint concentration of [X] of about 13 nM. When the system was perturbed by adding X, the concentration of X had returned close to its setpoint by the time the sample was returned to the fluorescence reader after adding the disturbances. This delay in measurement was no more than 10 minutes, and generally less, suggesting that the fast buffer had a response time of at most 10 minutes, significantly faster than the original buffer. As with the slower buffer, a slower response time of the fast buffer to the addition of competitor was observed as compared to the addition of X, consistent with the effects of toehold occlusion (FIG. 15).

### Example 8

Reporter Calibrations and Data Processing. Concentrations of X reported in FIGS. 3-4 were determined using a reporter complex that reacts reversibly with X, resulting in a change in fluorescence. The unreacted reporter has a quenched fluorophore, while the reacted form R_{F} has an unquenched fluorophore. The change in fluorescence thus reflects the concentration of X.

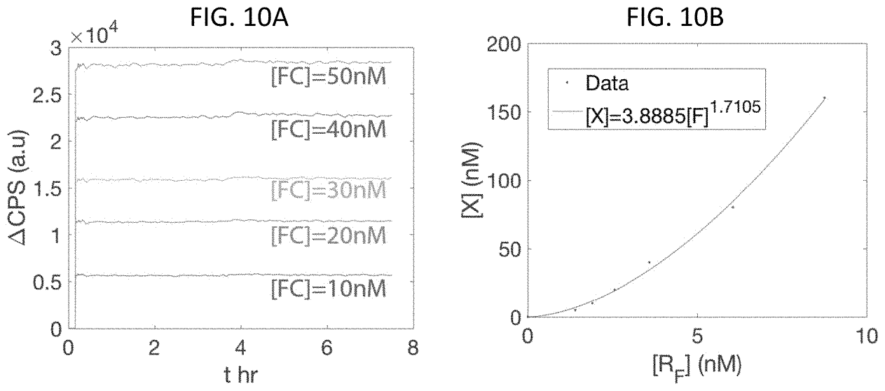

To determine [X] from raw fluorescence values a set of empirical calibration curves was measured. To create these curves, a time-dependent scaling factor ∝ (t) that relates the measured fluorescence intensity was first measured, in counts per second (CPS), to the concentration of unquenched reporter complex [R_{F}] in solution (see R_{F} in FIG. 3B) at a given time during an experiment. Time dependence of this factor results from fluctuations in the light or detector that affect all samples. To measure ∝ (t), 100 nM of Reporter was mixed with different concentrations of the Reporter's full complement (see Reaction Order Table above). This full complement (FC) reacts irreversibly with the Reporter, producing an unquenched product referred to as R_{F2}. Thus, the concentration of R_{F2} should be equal to the concentration of full complement that was added (FIG. 10A). It was assumed that the fluorescence of R_{F} is equal to the fluorescence if R_{F2}.

For each trajectory, ∝ was calculated as a function of time, which accounts for fluctuations in lamp intensity.

$$∝ ( t ) ≡ \frac{[ F u l l C o m p l e m e n t ]}{Δ R a w C P S ( t )}$$

ΔRawCPS(t) is the difference between the fluorescence intensity at time t and the fluorescence intensity before the Full Complement strand is added. The average c (t) was taken for five different full complement trajectories in an experiment. This factor was used to calculate the concentration of R_{F} in all other experiments as follows:

[R_{F}](t)≡ΔRawCPS(t)·∝(t)

Next, ∝ (t) was calculated separately every time an experiment was run, to take into account variations in lamp intensity that are specific to each experiment.

Next, the time-independent relationship was determined between [R_{F}] and [X]. About 100 nM of Reporter was mixed with different concentrations of X, which reacts reversibly with Reporter to separate the R_{F} and R_{Q} species. This data was then fit to a calibration curve, of the form [X]=c₁·([R_{F}])^{c}^{₂}, where c₁ and c₂ are the fit parameters (FIG. 10B). Separate curves were fit for each set of experiments run with different batches of reporter, to account for batch-to-batch variation.

With these transformations, [X] was then calculated in subsequent experiments as:

[X](t)=c₁·(ΔRawCPS(t)·∝(t))^{c}^{₂}

for the c (t), c₁ and c₂ determined for the given experiment.

Selecting a Reporter Toehold Length. To use a reporting reaction to measure the kinetics of buffering, it was necessary to ensure that the reporter equilibrates at least as fast as the buffering reaction. Verification that a reporter was chosen whose reaction rate was fast enough to measure the buffer by modeling both the buffer and reporter as simple bimolecular reaction using estimates for bimolecular reaction rate constants as a function of toehold length. Eqn 1 (Appendix A) was used for the buffer, and the equation below was used for the reporter:

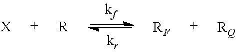

The kinetics of the buffering reaction was then compared to the kinetics of the reporting reaction when coupled to the buffering reaction with the goal of seeing that the rates of the two reactions were very close to one other. To further ensure that the reporting reaction would not place a significant load on the reporting reaction, a check of the simulation predicted was made to verify that the kinetics of the buffering reaction in the presence of the reporter would be similar to the kinetics of the same reaction without the reporting reaction.

The results of the simulation are shown in FIG. 11. For an 8 μM uniform buffer with a 0 nt source toehold and a 2 nt sink toehold, it was found that a reporter with a 5 nt toehold was sufficient to equilibrate on the same timescale as the buffer shown in FIG. 3A (see also FIG. 11A). In contrast, a 2 nt reporter toehold equilibrates much slower than the buffer (FIG. 11B). For the faster buffer with a 2 nt source and 4 nt sink toehold, the 5 nt reporter complex did appear to marginally slow the equilibration process somewhat, indicating that the fast buffer could be equilibrating even faster than the reporter's change in fluorescence would indicate (FIG. 11). In none of these idealized bimolecular models did the concentration of the reporter used in the experiments place a significant load on the equilibrium state of the buffering reaction.

The three-step model^{SI-R4} of DNA strand-displacement (DSD) offers a means to quantitatively estimate the kinetics of DSD reactions, to within an order of magnitude. It approximates each strand-displacement event as a series of three steps (i) toehold binding, (ii) branch migration, and (iii) toehold dissociation (FIG. 12).

Reaching different concentration set points by altering the concentration of Source, Initiator and Sink. To better under the experiments in FIG. 4 where the buffering reactions were characterized for different concentrations of either Source and Initiator or Sink, these same reactions were simulated using the three-state model. These simulations matched the experimental data in FIG. 4 to within an order of magnitude. The simulations predicted slightly lower steady-state concentrations than were observed in experiments and slightly higher rise times for low concentrations of Sink than were observed in experiments (FIG. 12).

### Example 9

Relaxation Time Slows Down when Reporter is Included. Three-step model simulations (following the same script as above) were used to examine the effect of the reporter on the buffer's relaxation time. By comparing simulations of the buffer's equilibration with and without the reporter present, the presence of the reporter may increase relaxation times (increase τ), and create a small dependence of the relaxation time constant τ on the initial concentrations of Source and Initiator (FIG. 13). These effects arise because it takes time for the buffer to generate or “charge up” the reporter and its intermediate products in the three-step model. Higher concentrations of Source and Initiator can generate X at a faster rate when the reporter is far from equilibrium, and thus can charge the reporter faster.

Toehold Occlusion. DNA strand displacement (DSD) reactions can slow down significantly due to toehold occlusion, the phenomenon in which one species temporarily binds to a complementary toehold on another species but cannot then fully displace the adjacent recognition domain. The rate at which toehold occlusion occurs increases with reactant concentration and toehold length, so it is particularly noticeable when there are high reactant concentrations involving long toeholds. In the DSD buffer design, the Initiator is present at high concentration and shares a long complementary 7 nt toehold with the Competitor complex (FIG. 15A) creating a potential for significant toehold occlusion involving the Initiator and Competitor. Initiator+Reporter and Initiator+R_{F} also have the potential for toehold occlusion, with a 5 nt toehold. Initiator+Sink may also have some occlusion interactions, as both species are present at high concentration, although they only share a short 2 nt toehold. All other occlusion interactions involve significantly lower reactant concentrations and/or smaller toeholds and may not be as significant in determining the buffer system's kinetics.

To estimate the degree to which toehold occlusion affects the magnitude and timing of the negative disturbances, three Three-Step Model simulations were run of the 8 uM uniform buffer being disturbed by 100 nM Competitor. In the first simulation, any occlusion effects were not included (FIG. 15B). In the second simulation, the relatively minor occlusion effects were included of the Initiator+Reporter and Initiator+R_{F} and Initiator+Sink (FIG. 15C). With these three occlusion effects turned on, the setpoint concentration was shifted down, but the disturbance was still able to deplete the concentration of X, after which the buffer restored the concentration of X close to the setpoint concentration. The effect of these types of occlusion should be able to be addressed by tuning the buffer with higher concentrations of source or initiator, or by increasing the forward rate constant to counteract the reduced setpoint. In the third simulation, the same toehold occlusions were included as in simulation 2, but also added the effect of the occlusion between initiator and competitor (FIG. 15D). It was observed that the 100 nM of competitor was unable to fully deplete the concentration of X, similar to what was observed in experiments (FIG. 5A). It was also observed that the recovery to the setpoint concentration was significantly slower than when this form of toehold occlusion was not present.

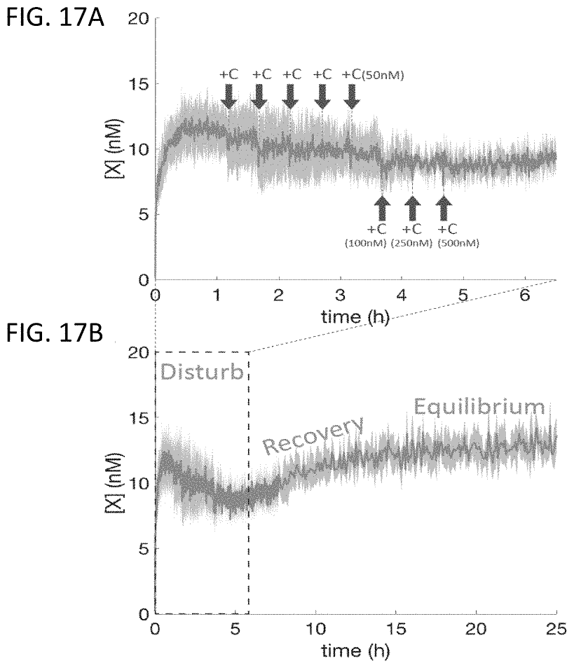

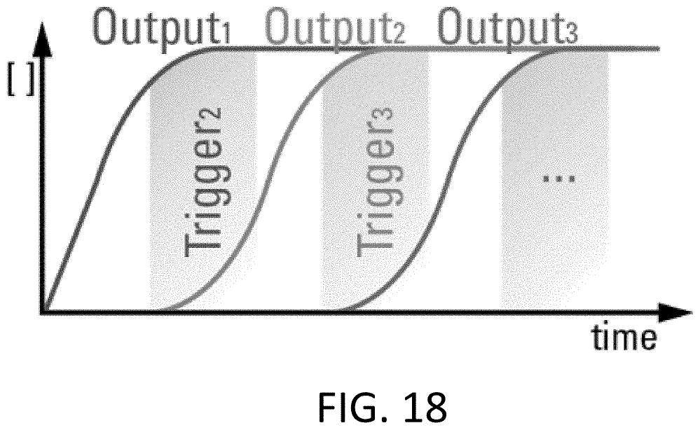

Negatively disturbing the 8 μM uniform fast buffer. The 8 μM uniform fast buffer to negative disturbances was tested by adding pulses of competitor (FIG. 17). A gradual relaxation back to equilibrium was observed. The slower speed of relaxation in response to the competitor than in response to X is consistent with the hypothesis that toehold occlusion prevents the competitor from reacting instantaneously with X. Effectively, toehold occlusion creates a transient load on the system until all of the competitor is consumed.

### Example 10

Embodiments of the present disclosure include a method to release an ordered series of output molecules in solution from a sequestered state. The mechanism of release consists of stages of paired reactions that first release an output molecule quickly (Eqn 1 below), and then slowly trigger the next reaction stage (Eqn 2 below). The large difference in the rate constants of these reactions ensures that each current stage will be essentially complete before the next stage is triggered.

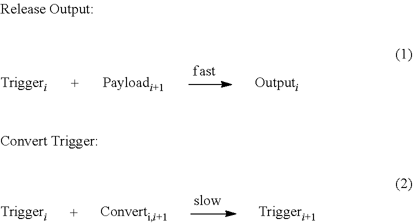

This system can either run asynchronously, or can be coupled with a central clocking mechanism that slowly generates Trigger₁ to control the pace of execution (Eqn 3 below).

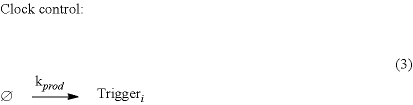

Embodiments of the present disclosure implement sequential release programs that can release different sequences of DNA using DNA strand-displacement (DSD) reactions. DSD reactions are designed interactions between short synthetic strands of DNA, in which an input strand binds to a partially double-stranded complex and displaces an incumbent output strand into solution. The reaction rate constants for DSD reactions are mediated by a short single-stranded DNA “toehold” domain. By varying the length of the toehold domain from 0 to 7 nucleotides, the rate constant can be tuned across six orders of magnitude at room temperature. Cascades of DSD reactions, in which the output of one reaction can serve as a reactant to a downstream process, can be used to implement a growing library of signal processing circuits, including amplifiers, Boolean logic gates, a neural network, an oscillator, a timer, and a feedback controller. Further, DSD circuits can control molecules other than DNA by designing their output to be aptamers, or sequences that can bind to proteins and small molecules.

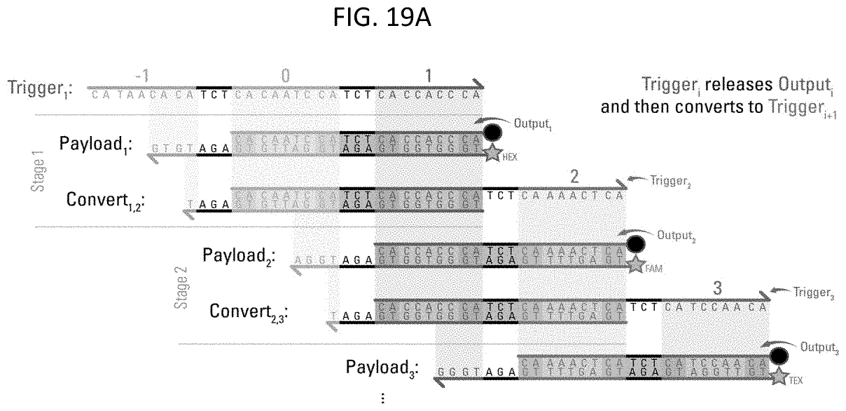

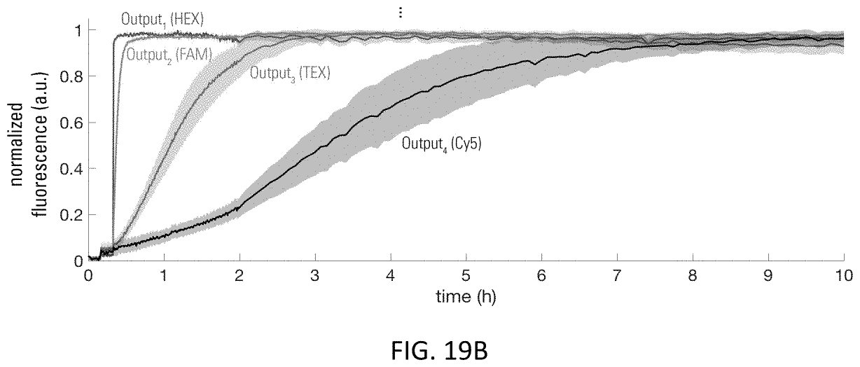

Sequential release cascade. To implement the reactions outlined in Eqn's 1-2 (above), a set of target Output strands of DNA was specified that were to be release in series. Each Output is initially sequestered in an inert state within a complex called the Payload. A set of Trigger strands is then designed to release the Outputs from the Payloads, following Eqn 1 (above). A 7 nt toehold was selected for the Trigger complex, which has the fastest rate constant (˜4 μM⁻¹s⁻¹) available to DSD reactions at room temperature. Next, a set of Convert reactions was designed to translate each Triggerᵢ molecule into the next Triggerᵢ₊₁ molecule in the series, following Eqn 2 (above), mediated by a slower 4 bp toehold (˜2·10⁻² μM⁻¹s⁻¹). To track the Outputs, a fluorescent modifier was appended to each of the Outputs, and a quencher to the bottom strands of the Payloads (FIG. 19A). This design uses the “leakless” DNA strand-displacement architecture to suppress unintended leak reactions directly between the Convert and Trigger complexes. This architecture imposes some sequence overlap between the Outputs from each stage; however, the sequence overlap can be eliminated with an additional translator reaction inserted after each stage.

To test whether the sequential release cascade releases Outputs in the sequential order, a four-stage cascade was prepared by mixing 25 nM of each Payload complex, together with 37.5·(4−i)nM of the Convert_{i,i+1} complexes for i=1, 2 or 3 to translate each Triggerᵢ to Triggerᵢ₊₁. Earlier stages require high concentrations of their convert complex to drive all of the remaining downstream stages. About 112.5 nM of the Trigger₁ strand was added to this mix to trigger the reaction cascade, and the resulting kinetics were tracked by measuring changes in fluorescence over time. The fluorescent Outputs were released in the designed order (FIG. 19B). The release rate decreased at every stage, as additional convert steps (of the form in Eqn 2 above) are required to translate Trigger₁ from the 1^{st} stage into Trigger on increasing i^{th} stages.

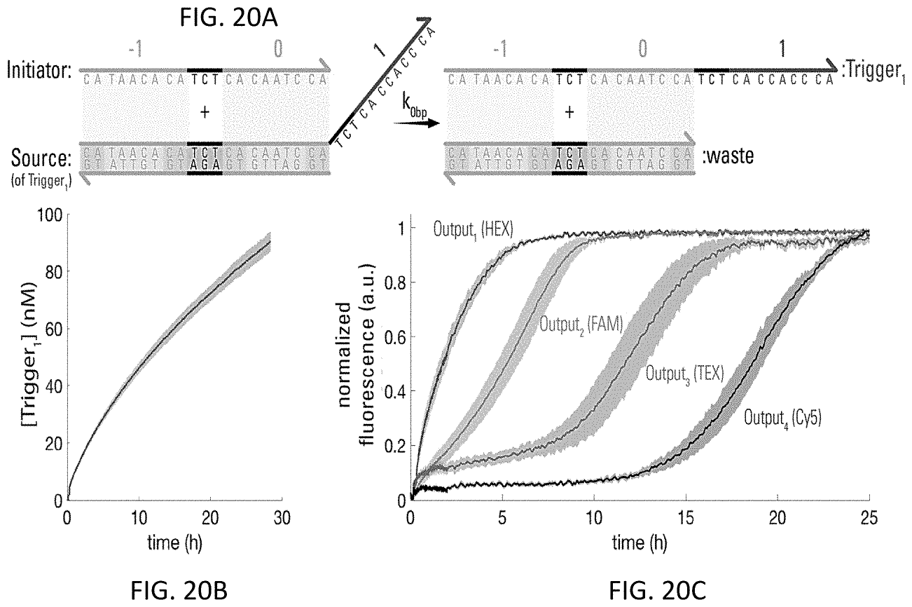

Clocking. To make the timing of the release events more uniform, an upstream clocking reaction was added that continuously produces Trigger₁ at a constant rate, replacing the large initial concentration of Trigger₁. In this manner, the clocking production reaction can be made into the rate-limiting step and thus control the pace of the reaction. The production reaction is performed by initially sequestering Trigger₁ within a high concentration Source complex. An Initiator strand reacts with the Source to displace Trigger₁ into solution (FIG. 20A), where it can then react with the sequential release cascade. By designing the reaction between Source and Initiator to have a very small rate constant, very little Source and Initiator is consumed on timescales of several days, and thus can approximate their concentrations as constant. This allows the net production rate to be treated as roughly a constant k_{prod} for the duration of the experiment (Eqn 1 above).

To verify that the clock circuit can sustain approximately linear release rates of the Trigger₁ molecule, Source and Initiator were mixed together at 1 μM in the presence of 300 nM Payload₁. By tracking increase in fluorescence, the rate at which Trigger₁ was released could be indirectly inferred (FIG. 20B).

Next, the clock reaction was coupled with the sequential release cascade to release the Outputs at regular intervals. About 1 μM Source and Initiator with 25 nM of each Payload complex, together with 37.5·(4−i)nM of each Convert_{i,i+1} complex. The same order of Output release events was observed, but now with a slower and more regular interval between the stages (FIG. 20C).

Branched Pathways & Conditional Statements. The circuits described above execute a linear release program with no branching, which is analogous to a computer program that does not have any conditional “if” statements. Every time they are run, they release the same molecules in the same hard-coded order. Including the capacity for conditional statements would allow for release programs to make decisions based on external factors sensed in solution, such as the presence of other signal strands of DNA. Therefore, the design of the Convert complexes was updated to create a conditional Convert complex, iConvert, which is initially inactive and can be conditionally activated through a reaction with a Deprotect strand (Eqn 4 below).

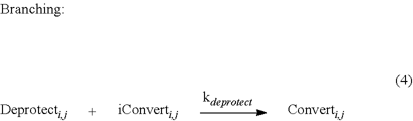

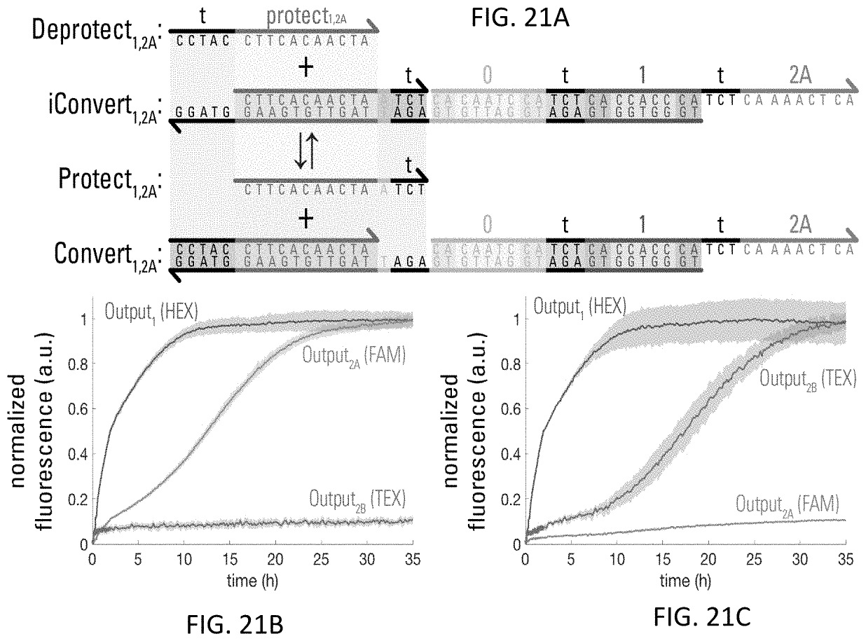

The toeholds on iConvert complexes are covered to prevent reactions with the trigger signals (FIG. 21A). A Deprotect strand is designed to expose the toehold on the inactive Convert complex, allowing it to react with the trigger. For simplicity, the deprotection reaction was approximated as irreversible, because the product activated convert complex is irreversibly consumed by Eqn 2 (above). This serves as a conditional statement of the form:
if(Deprotect_{i,j} is present){convert(Triggerᵢ to Triggerⱼ);}

Multiple inactive Convert complexes can be combined in the same solution to create conditional statements with more than one branching cases.

This conditional circuit was tested by preparing a two-stage sequential release circuit with a single Payload for the first stage and two different Payloads for the second stage. These species were referred as Payload₁, Payload_{2A} and Payload_{2B}. Two inactive Convert complexes iConvert_{1,2A} and iConvert_{1,2B} form separate branches to release the Outputs from their respective Payloads. The inactive Convert complexes can be activated by their Deprotect strands. Two batches of the clock circuit were mixed together (1 μM of Source and Initiator), 37.5 nM of iConvert_{1,2A} and iConvert_{1,2B}, and 25 nM of Payload_{2A} and Payload_{2B}. To one batch, 50 nM of Deprotect_{1,2A} (FIG. 20B) was added, and to the other batch 50 nM of Deprotect_{1,2B} (FIG. 20C) was added. In both cases, the Output for the activated branch was released, while the Output for the inactive branch was not significantly released.

### Example 11

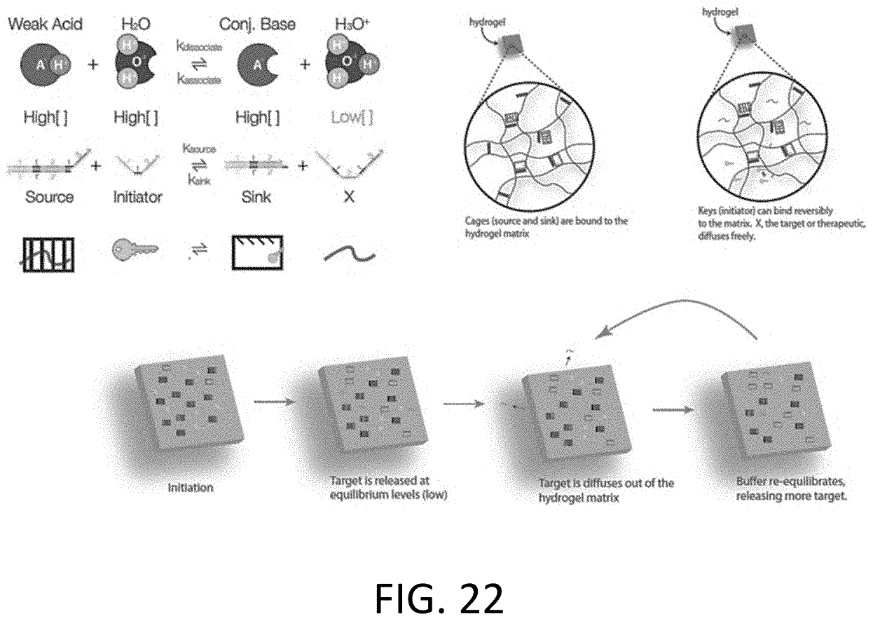

As shown in FIG. 22, embodiments of the present disclosure include the use of the oligonucleotide buffer compositions and systems disclosed herein with various drug delivery systems. In accordance with these embodiments, the oligonucleotide buffer compositions and systems disclosed herein can be incorporated into a drug deliver matrix for the controlled release of a constant concentration of a drug. In some embodiments, the drug delivery matrix can include hydrogel-based compositions. Hydrogels are materials composed of crosslinked hydrophilic polymer chains in water. By incorporating oligonucleotides into a hydrogel as crosslinks, the material properties of the hydrogel can be manipulated dynamically by then adding various oligonucleotides that alter crosslink conformation, break up the hydrogel structure, or help create new crosslinks. Crosslinks within hydrogel matrices can be reversibly dissociated by adding a strand complementary to one of the crosslink strands, such as a target polynucleotide described herein. Hydrogel with DNA crosslinks can also be stiffened or softened by adding oligonucleotides that alter the conformation of crosslinks between a double-stranded state or a partially single-stranded conformation via DNA strand-displacement reactions, as described herein.

In accordance with these embodiments, oligonucleotide buffer compositions and systems can include aptamer sequences of DNA that non-covalently bind specifically to small molecules such as, but not limited to, ATP, cocaine, therapeutic drugs and other small molecules, metal ions, proteins and peptides. Aptamers or aptamer complexes that contain aptamer sequence motifs can induce a change in the conformation of another oligonucleotide strand or complex upon binding of the target small molecule (e.g., within a hydrogel drug delivery matrix). In many cases, this conformational change can release a strand of DNA in the presence of the target molecule, which can then serve as a DNA circuit input. Aptamers or aptamer complexes that contain aptamer sequence motifs can also enable multi-stranded DNA complexes to participate in downstream strand-displacement reactions by exposing a toehold domain on the complex or removing clamp domains that inhibit invading strands from binding to the complex.

Output strands released by DNA circuits can also direct the capture or release of molecules from an aptamer by binding to the aptamer and changing its state. Bahdra and Ellington modified the fluorescent RNA Spinach aptamer to fold into an inactive state in which it could not associate with its target molecule (DFHBI fluorophore). Hybridization with a trigger strand of DNA refolds the aptamer into an active state in which it successfully binds its target (#37). Lloyd et al started with aptamers bound to their targets, and used complementary strands of DNA, called kleptomers, to bind to and displace aptamers from their targets. This technique was demonstrated with both a Broccoli aptamer, which binds to DFHBI, and an aptamer for RNA polymerase, which in binding prevents the RNA polymerase from transcribing (FIG. 3b, #38). Aptamers have also been integrated into reconfigurable DNA nanostructures, for instance they have been used as binding sites for target molecules at the end of DNA nanotweezers (#39). When a trigger strand of DNA opens the tweezers, the two aptamer binding sites at the ends of the tweezer separate. Because binding of the target protein requires interaction with both binding sites, this conformational change releasing the protein target. The tweezer nanostructure thus allows a strand of DNA that is not itself an aptamer binding sequence or its complement to direct the release of a target molecule.

In some embodiments, hydrogels crosslinked with oligonucleotides can exhibit bulk elastic moduli ranging from tens of Pascals (Pa) to about ten kPa, and can exhibit swelling upon softening, with volumetric swelling ratios up to about 25% between the stiff and soft states. In some embodiments, photolithographic patterning can be used to form multi-domain oligonucleotide-crosslinked hydrogels where each domain can be swollen independently by the addition of different hairpin fuel strands of DNA to the surrounding medium. The differential swelling of domains within a hydrogel can cause bending or curling, which can be translated into mechanical actuation.

TABLE 2
Sequences.

| | | | SEQ |
|---|---|---|---|
| | | IDT | ID |
| Strand | Sequences | Purification | NO: |
| Signal | CA TAACA CA TCT CA CAATC | PAGE | 1 |
| (X) | CA TCT CA CCACC CA | | |
| Source | GATG GATTG TG AGA TG | PAGE | 2 |
| Bottom | TGTTA TG | | |
| Initiator | CA TAACA CA TCT CA CAATC | PAGE | 3 |
| | CA | | |
| Reporter | CAATC CA TCT CA CCACC CA | HPLC | 4 |
| Top (R_{Q}) | TCT CA/3IABkFQ/ | | |
| Reporter | /56-FAM/TG AGA TG GGTGG | HPLC | 5 |
| Bottom | TG AGA TG GATTG TG AGA | | |
| (R_{bF}) | | | |
| Reporter | TCT CA CAATC CA TCT CA | Standard | 6 |
| Full | CCACC CA TCT CA | desalting | |
| Complement | | | |
| Competitor | CA CAATC CA TCT CA CCACC | Standard | 7 |
| Top | CA CT | desalting | |
| Competitor | AG TG GGTGG TG AGA TG | Standard | 8 |
| Bottom | GATTG TG AGA TG | desalting | |
| Signal₂ | CAA TCT ACA TCT CAA CAC | PAGE | 9 |
| (X₂) | TCA TCT CAT TCC TCA | | |
| Source₂ | GAT GAG TGT TGA GAT GTA | PAGE | 10 |
| Bottom | GAT TG | | |
| Initiator₂ | CAA TCT ACA TCT CAA CAC | PAGE | 11 |
| | TCA | | |
| Reporter₂ | CA ACACT CA TCT CA TTCCT | HPLC | 12 |
| Top | CA TCT CA/3IABkFQ/ | | |
| Reporter₂ | /5HEX/TG AGA TG AGGAA TG | HPLC | 13 |
| Bottom | AGA TG AGTGT TG AGA TG | | |
| Reporter₂ | CA TCT CA ACACT CA TCT | Standard | 14 |
| Full | CA TTCCT CA TCT CA | desalting | |
| Complement | | | |
| Initiator | C CA TAACA CA TCT CA | PAGE | 15 |
| (Fast) | CAATC | | |
| Source | GA TG GATTG TG AGA TG | PAGE | 16 |
| Bottom | TGTTA TG GT | | |
| (Fast) | | | |

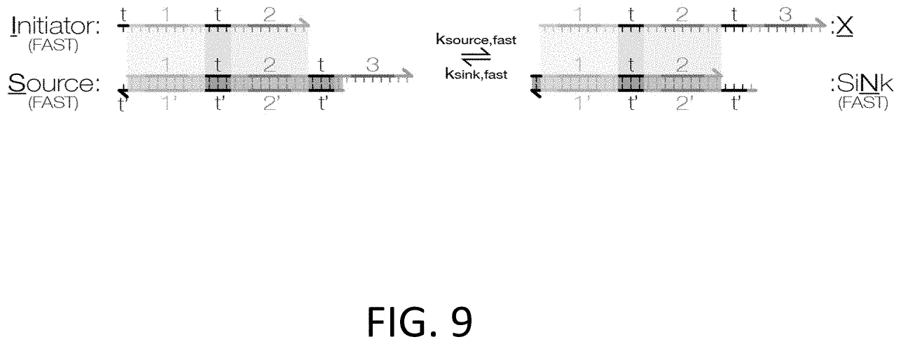

Sequences for each strand used in the present disclosure are listed in Table 2 (above). Secondary structures were verified for each complex using NuPack^{SI-R2}. The domain level structure of the fast buffer (with toeholds extended from 0 nt to 1t for the Source, and 2 nt to 4 nt for the Sink) is illustrated in FIG. 9.

### Example 12

Seeded DNA nanotube design and growth at different seed and tile concentrations. There are three growth regimes for DNA nanotubes that are dictated by the initial concentration of free tiles. At high initial tile concentrations (>200 nM), seeds are not required for nanotube growth and DNA tiles can spontaneous nucleate to form nanotubes that grow and/or join together end-to-end in an uncontrolled fashion (FIG. 23C, I.). At lower initial tile concentrations (100-200 nM), seeds are required for significant nanotube growth to occur and DNA nanotubes nucleate and grow specifically from seeds (FIG. 23C, II). At even lower initial tile concentrations (<100 nM) the tile concentration is below the critical concentration for growth (FIG. 23C, III.). In a batch growth process, nanotube growth consumes free tiles and eventually the free tile concentration approaches the critical tile concentration, halting growth.

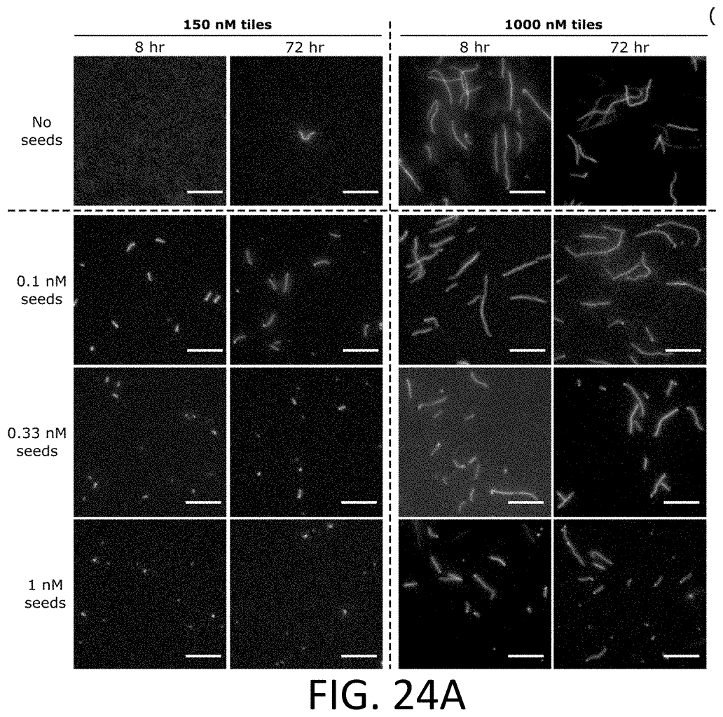

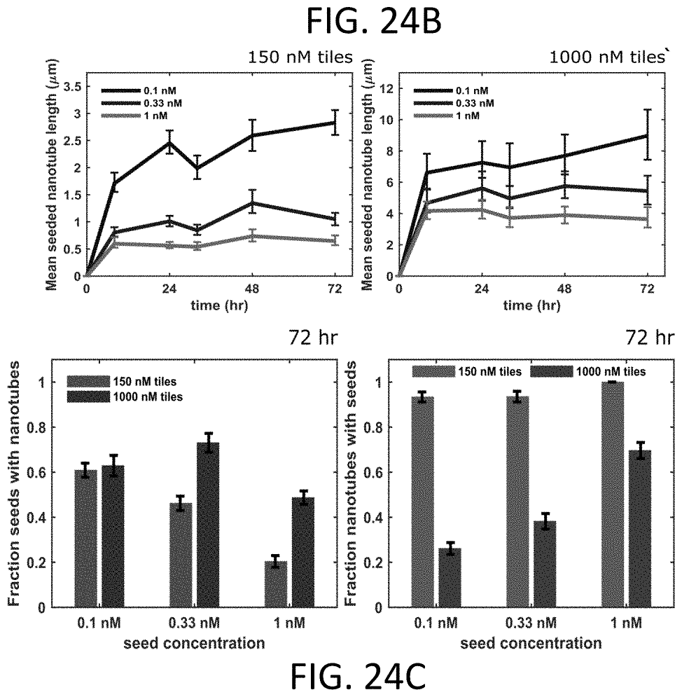

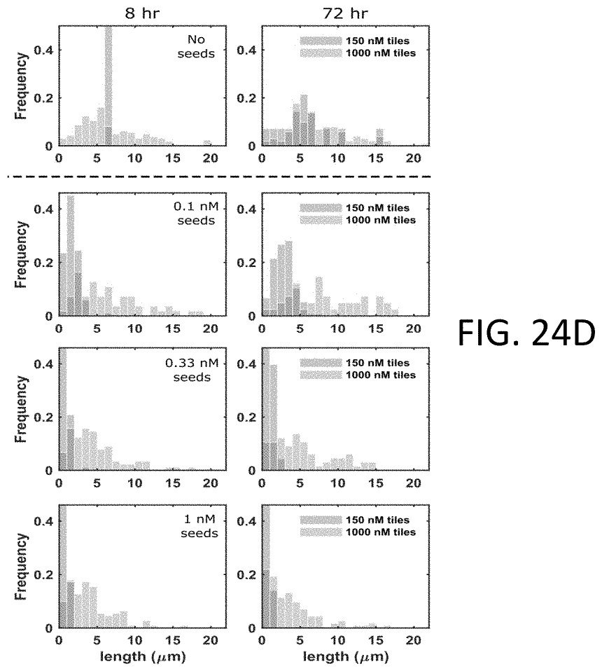

To grow nanotubes in the seeded growth tile concentration regime, relatively low seed concentrations are typically used (10-100 pM) to ensure significant nanotube growth. Increasing the seed concentration results in considerably less growth as the same total amount of tiles will be distributed across more seeds. For example, significant nanotube growth can be achieved with a fixed 150 nM tiles (a tile concentration in the seeded growth regime) when 100 pM of seeds are used. However, increasing the seed concentration resulted in significantly less nanotube growth (FIG. 24A, left). Both the mean nanotube length (FIG. 24B, left) and the fraction of seeds nucleating nanotubes (FIG. 24C, left) decreased with increasing seed concentrations. Further, active nanotube growth slows down and stops after 8 to 24 hours (FIG. 24C, left). Since these effects are due to tile depletion during nanotube growth, one route to enhance the growth at higher seed concentrations is to increase the free tile concentration. Growth with 1000 nM of tiles resulted long nanotubes (FIGS. 24A-24B, right) and high fractions of seeds nucleating nanotubes (FIG. 24C, left) at all the seed concentrations tested. But 1000 nM tiles pushes the system into an unseeded growth regime and nanotubes spontaneously nucleated without seeds (FIG. 24A, right) and even with seeds present a large fraction of nanotubes nucleated on their own (FIG. 24C, right). Spontaneous nucleation at high tile concentrations prevents control over when and where nanotubes are grown and also produces much wider distributions in nanotube lengths compared to purely seeded nanotube growth due to nanotubes joining end-to-end during the growth process (FIG. 24D). Additionally, seeds are not required for nanotube growth (FIG. 24A, right) and the same number of nanotubes, mean nanotube length, and length distributions are observed both with and without seeds. These results indicate that seeds do not direct the growth process in this regime and suggest that many seeded nanotubes are likely the product of seeds binding to nanotubes that nucleated and grew spontaneously. Additionally, spontaneous nucleation at high tile concentrations also results in rapid tile depletion across all of the seed concentrations so active nanotube growth still ceased after 8 hours (FIG. 24B, right).

The results in FIG. 24 demonstrate the limited range over which controlled seeded nanotube growth can occur. In the seeded growth regime, there is limited chemical potential for growth since the tile concentration is only slightly above the critical concentration and in the unseeded growth regime, there is an initially high chemical potential for growth, but spontaneous nucleation quickly depletes it. An ideal system for nanotube growth would possess a high chemical potential but control how this potential is used during growth. This could be achieved by maintaining the free tile concentration in the seeded growth regime allowing nanotube growth to be sustained for longer time periods and over a broader range of seed concentrations (FIG. 23D, top). Such a system would require a feedback control mechanism that could adjust the free tile concentration to maintain a specific tile concentration setpoint during the growth process (FIG. 23D, bottom).

### Example 13

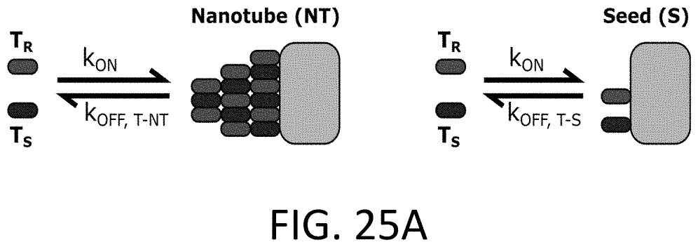

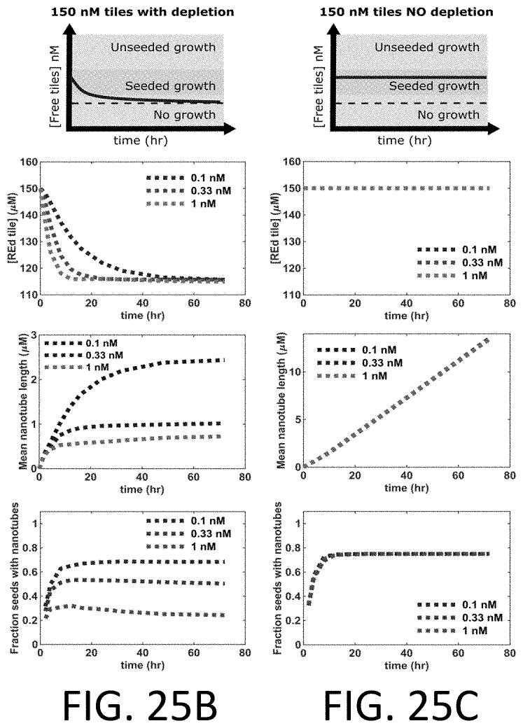

A tile concentration buffering scheme to sustain DNA nanotube growth. To understand the seeded nanotube growth process and investigate how growth proceeds without tile depletion, a stochastic kinetic model of seeded nanotube growth was built. The model is based on tiles reversibly binding to either a nanotube or seed growth face. The presence of a nucleation barrier for the seed results in two tile off rates, one for tiles bound to nanotubes and another for tiles bound to seeds (FIG. 25A). The on rate for tile binding has been determined to be between 10⁵-10⁶ M⁻¹ s⁻¹ and the tile off rates are related to the free energy of tile binding (ΔGᵢ) through Eq. 1, where i refers to either the tile-seed or tile-nanotube interaction, R refers to the gas constant, and T refers to the absolute temperature.

$${k}_{O F F , i} = {k}_{O N} exp ( - \frac{Δ {G}_{i}}{R T} ) \qquad ( 1 )$$

Using the 150 nM tile growth results in FIG. 24, the ΔG of tile binding to nanotubes was calculated and seeds in the kinetic model were used to recapitulate the experimental results (FIG. 25B). These values were then used to simulate seeded nanotube growth without any tile depletion. Without tile depletion, nanotube growth is predicted to proceed indefinitely at the same rate irrespective of seed concentration. Further, the fraction of seeds that nucleate nanotubes is predicted to reach the same maximum for all the seed concentrations (FIG. 25C). This fraction is less than 1 because a fraction of the seeds will have defects that render them inactive. The kinetic model considers 25% of the seeds to be inactive, consistent with other experimental results. In contrast, growth with tile depletion resulted in a decreasing fraction of seeds nucleating nanotubes with increasing seed concentrations (FIG. 25B), consistent with the experimental results (FIG. 24). These simulation results indicate that mitigating tile depletion should sustain DNA nanotube growth over a broad range of seed concentrations.

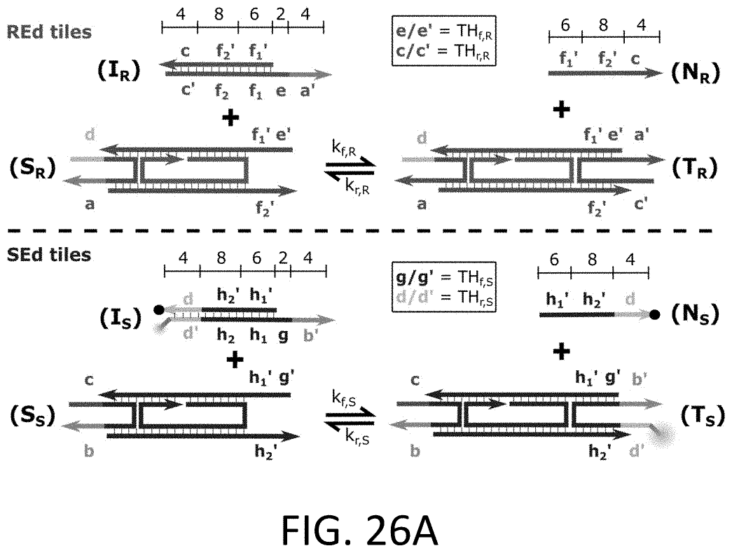

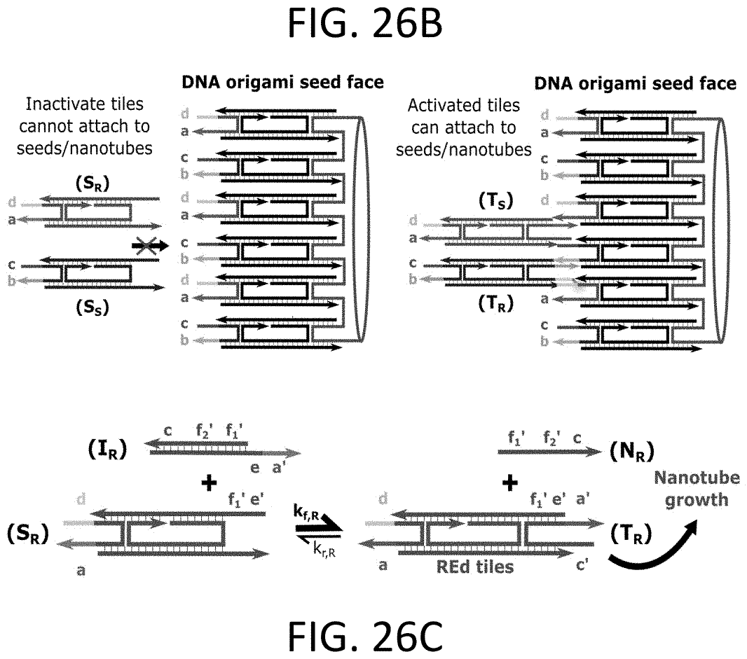

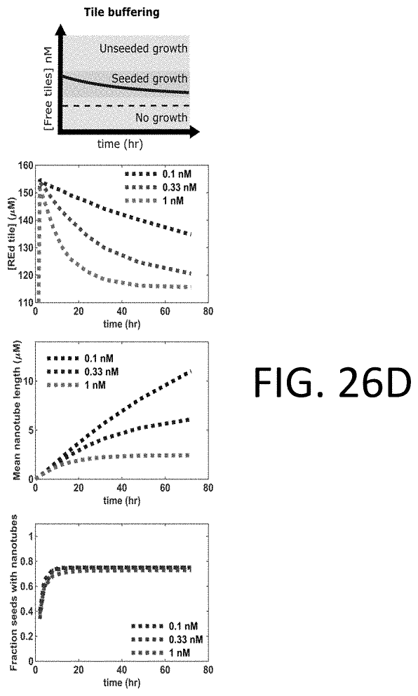

To implement a chemical feedback control mechanism that could mitigate tile depletion during nanotube growth, a reversible tile production reaction was designed via DNA strand displacement. The reaction network is composed of inert Source tiles (S_{R} or S_{S}) that react with Initiator complexes (I_{R} or I_{S}) via a toehold-mediated strand displacement reaction to create active tiles (T_{R} or T_{S}). A Sink strand (N_{R} or N_{S}) that reacts with the active tiles reverses the tile production reaction (FIG. 26A and eq. 2).

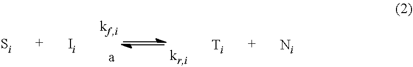

Since the tile production reaction is reversible, the system can be set to equilibrate to a specific active tile concentration by tuning the forward and reverse reaction rate constants and the concentrations of the Source, Initiator, and Sink species (eq. 3).

$${[ {T}_{i} ]}_{e q} = \frac{{k}_{f , i}}{{k}_{r , i}} \frac{{{[ {S}_{i} ]}_{e q} [ {I}_{i} ]}_{e q}}{{[ {N}_{i} ]}_{e q}} = {K}_{eq , i} \frac{{{[ {S}_{i} ]}_{e q} [ {I}_{i} ]}_{e q}}{{[ {N}_{i} ]}_{e q}} \qquad ( 3 )$$

The reaction rate constants (k_{f} and kᵣ) can be set (within an order of magnitude) by the length of the single-stranded toehold domains that facilitate the strand displacement processes (TH in FIG. 26A). The forward reaction was designed to initiate via a 2-base toehold (corresponding to a theoretical k_{f} of roughly 1×10² M⁻¹s⁻¹) and the reverse reaction to initiate via a 4-base toehold (corresponding to a theoretical kᵣ of roughly 1×10⁴ M⁻¹s⁻¹). These rate constants should produce a K_{eq} of roughly 0.01 which allows for relatively high concentrations of Source, Initiator, and Sink (>1 μM) to be used to set the active tile concentration around 100 nM where seeded growth occurs (eq. 3). These reaction rate constants should also be larger enough to allow for fast equilibration and response.

The reaction network is designed so that only active tiles can attach to a seed face or the face of a growing DNA nanotube. Thus, tile incorporation into a nanotube sequesters the toehold on the tile that facilitates the reverse reaction (FIG. 26B), effectively removing the active tile from the reaction network. Since the tile production reaction is reversible, by Le Chatelier's principle, as tiles are incorporated into nanotubes the reaction network will produce more active tiles to resist a change in the equilibrium tile concentration (FIG. 26C). This system resembles a pH buffer which shifts the equilibrium of a weak acid and its conjugate base to resist changes in pH. A similar DNA strand displacement buffering scheme has been successfully developed to buffer the concentration of specific DNA strands. Thus, this process was termed tile concentration buffering. Tile concentration buffering works by maintaining a high chemical potential in inert chemical species that are only converted into active species as they are needed for growth.

To determine whether tile concentration buffering could sustain DNA nanotube growth over a broad range of seed concentrations, stochastic nanotube growth simulations were conducted using concentrations of the Source, Initiator, and Sink species that would result in an initial active tile concentration of roughly 150 nM. From the simulations, the active tile concentration is predicted to decrease at a much slower rate with buffering (FIG. 26D, top plot) compared to growth with tile depletion (FIG. 25B, top plot), especially at the lower seed concentrations. Significant growth is predicted for all the seed concentrations with mean seeded nanotube lengths 4-6 times (FIG. 26D, middle plot) those predicted during growth with tile depletion (FIG. 25B, middle plot). Further, buffering is predicted to produce the same maximum fraction of seeds that nucleate nanotubes across all seed concentrations (FIG. 26D, bottom plot) as growth without tile depletion (FIG. 25C, bottom plot). These simulations suggest that tile concentration buffering should sustain nanotube growth over a broad range of seed concentrations and for longer periods of time.

### Example 14

Tile buffering enables growth at high seed concentrations and extends active growth time. To test whether tile buffering could enhance seeded nanotube growth, the concentration of the Source and Initiator complexes were fixed at a high concentration (5.5 μM each) and varied the concentration of the Sink strands to set the active tile concentrations to a range of different equilibrium values (between roughly 150-225 nM based on the theoretical K_{eq} value for the tile buffering reactions. Nanotube growth was then assessed with tile concentration buffering over a range of seed concentrations as well as without seeds present. It was found that a Sink concentration of 1.25 μM produced the best growth results, exhibiting significant nanotube growth over all the seed concentrations tested and hardly any growth without seeds present. These reaction conditions (Source, Initiator=5.5 μM, Sink=1.25 μM) were then used to further characterize nanotube growth with buffering.

Nanotube growth with tile concentration buffering resulted in sustained nanotube growth over 72 hours and produced seeded nanotubes 5-10 fold longer than growth with a fixed 150 nM of tiles across all the seed concentrations tested (FIGS. 27A-27B). Buffering also resulted in fairly monodispersed nanotube lengths throughout the growth process (FIG. 27C) and produced a high fraction of nanotubes attached to seeds (FIG. 27D, top), indicating that growth is initiated primarily from the seeds. Nanotube growth with tile concentration buffering also resulted in the maximum fraction of seeds nucleating nanotubes across all the tested seed concentrations compared to growth with 150 nM tiles where the fraction of seeds that nucleated nanotubes decreased with increasing seed concentration (FIG. 27D, bottom). These results are in line with the simulation results in FIG. 26D.

The total concentration of tiles that were incorporated into nanotubes during the growth process was quantified and it was found that, at the highest seed concentration, tile buffering resulted in nearly 20-fold more tiles being incorporated into nanotubes compared to growth with a fixed 150 nM tiles. For 150 nM tiles samples roughly 70 nM of tiles were incorporated into the nanotubes while growth with buffering at 1 nM of seeds incorporated >1300 nM of tiles into nanotubes (FIG. 27E). Buffering was able to produce nanotubes with similar mean lengths to growth with 1000 nM tiles but produced much more monodispersed length distributions and higher fractions of nanotubes with seeds since growth primarily occurred from the seeds. Further, buffering sustained active growth over the whole 72-hour experiment while growth with 1000 nM tiles ceased after 8 hours.

### Example 15

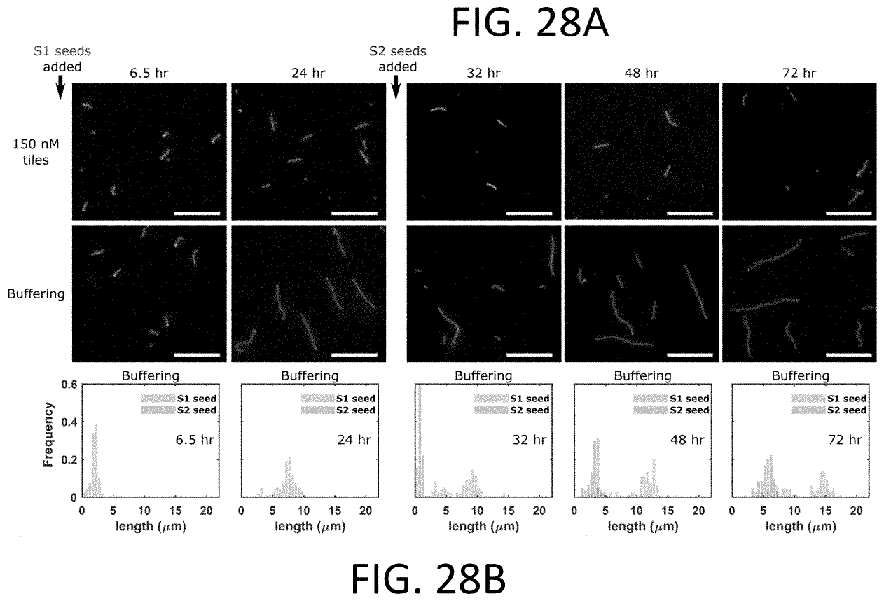

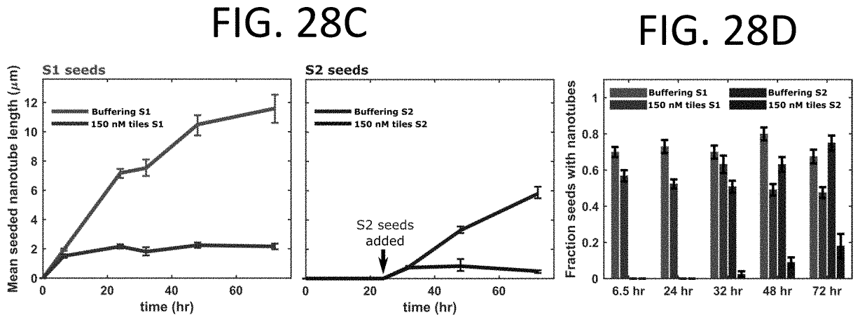

Tile buffering adapts to temporal changes in growth demand. Since tile concentration buffering is able to sustain active nanotube growth for 72 hours over a broad range of seed concentrations (FIG. 27), buffering should be able to adapt to temporal changes in growth demand, such as an increase in the concentration of seeds during the growth process. The capability to adapt to increases in growth demand could be important for building hierarchical structures where new growth sites are introduced or activated sequentially to build the final structure. For example, microtubule growth and branching is important during neuron development to build hierarchical axon networks. To investigate whether buffering could sustain nanotube growth through changes in growth demand, the buffering components were initially mixed with 0.1 nM of seeds (S1 seeds) and tracked nanotube growth for 24 hours. An additional 0.1 nM of seeds labeled with a different fluorescent tag (S2 seeds) was then added and tracked growth for another 48 hours. Conducting this experiment with a fixed 150 nM tiles, it was found that growth only occurred from the S1 seeds. In contrast, buffering resulted in nanotube growth from both the S1 seeds and the S2 seeds (FIG. 28A), resulting in unique length distributions for each seed type (FIG. 28B). With buffering, sustained growth was observed for both seeds over the course of the 72 hour experiment, while nanotube growth stopped after 24 hours for samples with a fixed 150 nM (FIG. 28C) or 1000 nM tiles. Further, buffering resulted in high fractions of seeds nucleating nanotubes for both seed types while hardly any nucleation was observed for the S2 seeds in the sample with a fixed 150 nM tiles (FIG. 28D). These results demonstrate that the buffering reaction is able to adapt to temporal changes in growth demand.

### Example 16

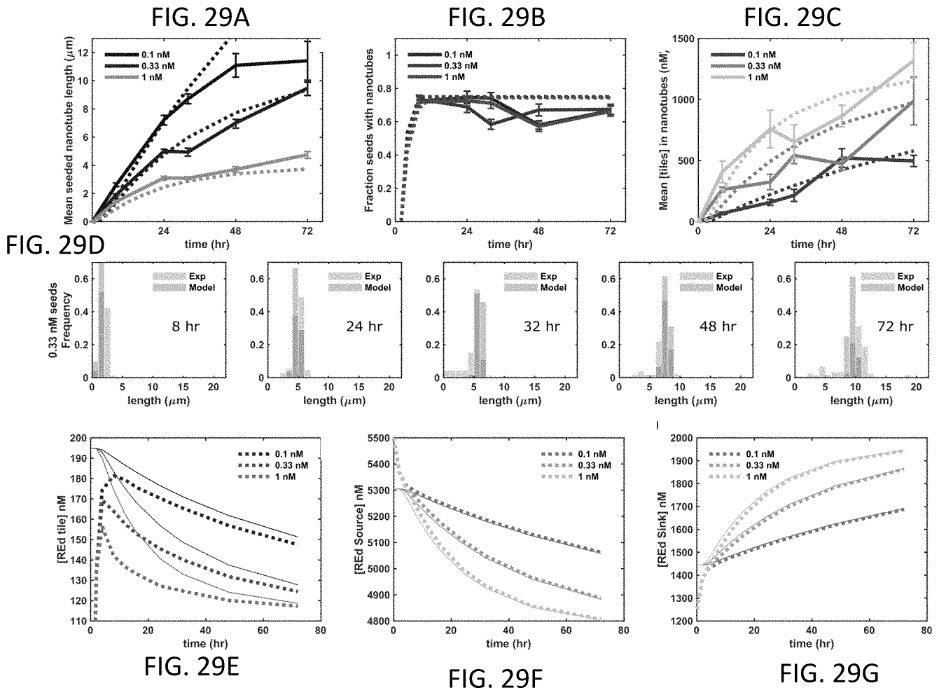

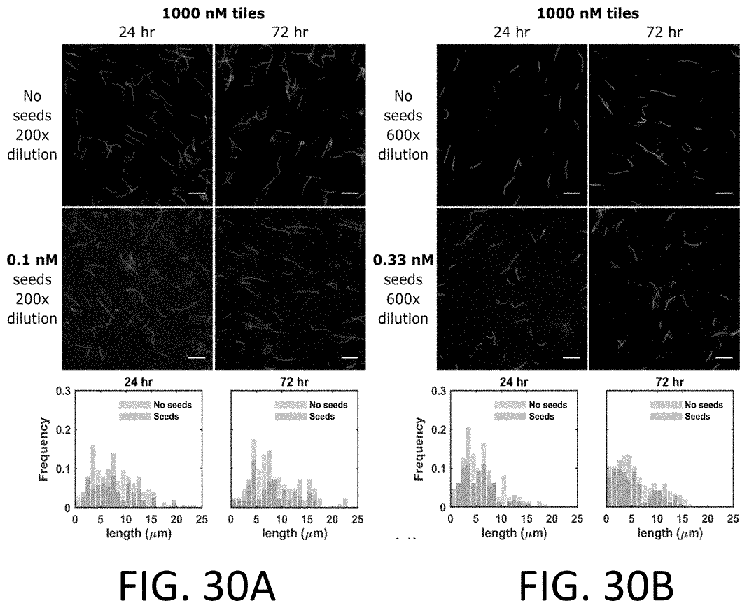

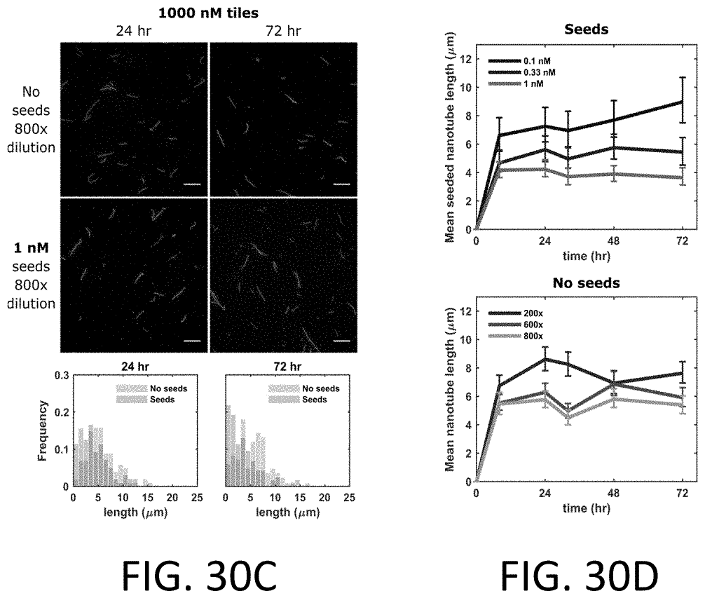

Modeling the coupled nanotube growth and tile concentration buffering. A quantitative kinetic model of the coupled nanotube growth and tile concentration processes was built to better understand how these systems. To better recapitulate the experimental nanotube growth results, the reaction rate constants were adjusted in the stochastic kinetic model of nanotube growth with tile concentration buffering (FIGS. 29A-29D). This model was then used to investigate how the tile buffering species varied over the course of the reactions. The simulations predicted that the active tile concentration will decrease over the course of the experiments (FIG. 29E). This is a result of the equilibrium setpoint of the tiles decreasing during nanotube growth. As tiles are consumed during growth and replenished by the buffering reaction network, the concentration of the buffering species changes, shifting the equilibrium tile concentration. Each tile that is produced by the buffer depletes a Source and an Initiator complex (FIG. 29F) and produces a Sink strand (FIG. 29G) and from Equation 3, all of these concentration changes will decrease the equilibrium tile concentration. Thus, nanotube growth will eventually halt once the equilibrium tile concentration of the reaction network drops to the critical tile concentration. The higher the seed concentration, the faster the buffer will reach this point (FIG. 29E).

The buffering reaction rate constants (k_{f} and kᵣ) that best recapitulated nanotube growth results were two orders of magnitude lower than predicted by the lengths of the toeholds used for the reactions. This could be because both of the buffering reactions require a strand to switch helices on the core of the tiles during the branch migration process. The simulations showed that the slower than predicted reaction rate constants cause the buffering reaction to be slower than nanotube growth and the active tiles cannot be replenished as fast as they are being consumed (FIG. 29E). Additional simulations with faster buffering reactions indicated that the slower buffering reactions slow down nanotube growth, but the two systems should ultimately reach similar final states after 72 hours.

Since changes in the concentrations of the buffering species during growth limits how long growth can occur, nanotube growth was simulated with either 2-fold or 5-fold higher initial Source and Initiator concentrations to see how much longer this would extend growth. In these simulations Sink concentrations were selected that would result in the same initial equilibrium tile concentrations as the buffer tested experimentally. These simulations showed that increasing the concentrations of the buffering species increases buffering capacity, predicting longer nanotubes and higher concentrations of tiles incorporated into nanotubes than the buffer tested experimentally. However, even with 5-fold more initial Source and Initiator complexes, the equilibrium tile concentration still significantly decreases during growth with high seed concentrations, so it is likely that particularly high concentrations (>100 μM) of the buffering species would be required to completely mitigate tile depletion. Growing nanotubes with such high concentrations of the buffering species could have practical limitations because it was found that the Source and Initiator complexes can interfere with nanotube growth.

### Example 17

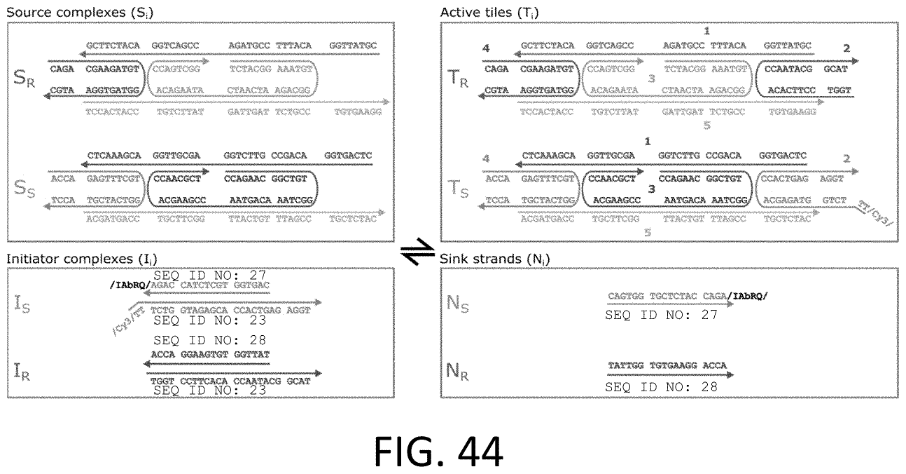

DNA tile and tile buffering components are shown in FIG. 44, and the sequences used are provided in Table 3.

TABLE 3
Sequences.

| | | SEQ |
|---|---|---|
| Strand | | ID |
| name | Sequence | NO: |
| T_{R} - 1 | CGTATTGGACATTTCCGTAGACCGACTGGACATCTTCG | 17 |
| T_{R} - 2 | TGGTCCTTCACACCAATACGGCAT | 18 |
| T_{R} - 3 | TCTACGGAAATGTGGCAGAATCAATCATAAGACACCAG | 19 |
| | TCGG | |
| T_{R} - 4 | CAGACGAAGATGTGGTAGTGGAATGC | 20 |
| T_{R} - 5 | TCCACTACCTGTCTTATGATTGATTCTGCCTGTGAAGG | 21 |
| T_{S} - 1 | CTCAGTGGACAGCCGTTCTGGAGCGTTGGACGAAACTC | 22 |
| T_{S} - 2 | /5Cy3/TTTCTGGTAGAGCACCACTGAGAGGT | 23 |
| T_{S} - 3 | CCAGAACGGCTGTGGCTAAACAGTAACCGAAGCACCAA | 24 |
| | CGCT | |
| T_{S} - 4 | ACCAGAGTTTCGTGGTCATCGTACCT | 25 |
| T_{S} - 5 | ACGATGACCTGCTTCGGTTACTGTTTAGCCTGCTCTAC | 26 |
| N_{R} | TATTGGTGTGAAGGACCA | 27 |
| N_{S} | CAGTGGTGCTCTACCAGA/3IAbRQSp/ | 28 |

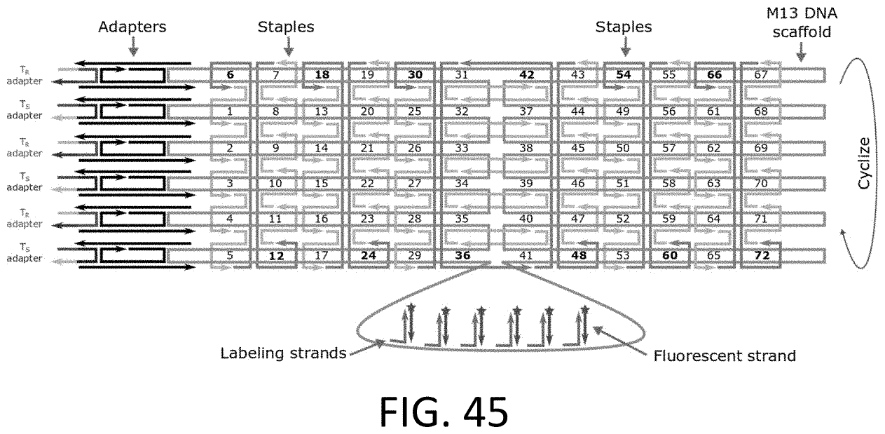

The design of the DNA origami seed was adopted from previous studies. A seed is composed of a scaffold strand (M13mp18 DNA (7,240 bases) purchased from New England Biolabs), 72 staple strands, and 24 adapter strands (strands on the adapters that possess the tile sticky end sequences). Dark staples in FIG. 45 direct the structure to cyclize into a cylinder.

The fluorescent labeling scheme for the origami seed was as described previously. Briefly, the unused M13 DNA on the termini was used as a binding site for one hundred unique DNA strands (labeling strands). Each labeling strand was complementary to a portion of the unfolded M13 at its 5′ end and had the same 15-base sequence at its 3′ end. The 15-base sequence at the 3′ end of each labeling strand served as a binding site for a DNA strand that was modified with a fluorescent tag at its 5′ end (either atto488 or atto647).

Sequences of DNA origami staple strands are shown in Table 4. All strands ordered unpurified from IDT. Numbers beside strand names correspond to the numbered staples in the diagram provided below. As previously described, hairpins (highlighted in red) were incorporated into the staples to induce a directional preference for cyclization.

TABLE 4
Sequences.

| | | SEQ |
|---|---|---|
| | | ID |
| Strand Name | Sequence | NO: |
| (1) T_5R2F_HP | 5′ | 29 |
| | TGAGTTTCAAAGGAACGTCCACCGTTTTC | |
| | GGTGGACTTAACTAAAGATCTCCAA 3′ | |
| (2) T_5R4F_HP | 5′ | 30 |
| | AAAAAAGGCTTTTGCGGTGGTCCGTTTTC | |
| | GGACCACTTGGATCGTCGGGTAGCA 3′ | |
| (3) T_5R6F_HP | 5′ | 31 |
| | ACGGCTACAAGTACAACTCGGCACTTTTG | |
| | TGCCGAGTTCGGAGATTCGCGACCT 3′ | |
| (4) T_5R8F_HP | 5′ | 32 |
| | GCTCCATGACGTAACACGGATCGCTTTTG | |
| | CGATCCGTTAAGCTGCTACACCAGA 3′ | |
| (5) T_5R10F_HP | 5′ | 33 |
| | ACGAGTAGATCAGTTGCACCGCTGTTTTC | |
| | AGCGGTGTTAGATTTAGCGCCAAAA 3′ | |
| (6) T_5R12F_CYC_HP | 5′ | 34 |
| | GGAATTACCACCACCCGTGAGGCGTTTTC | |
| | GCCTCACTTTCATTTTCCGTAACAC 3′ | |
| (7) T_5R2E_HP | 5′ | 35 |
| | GAGAATAGGTCACCAGCGGAACCGTTTTC | |
| | GGTTCCGTTTACAAACTCCGCCACC 3′ | |
| (8) T_5R4E_HP | 5′ | 36 |
| | AAAGGCCGCTCCAAAACCGTGGCGTTTTC | |
| | GCCACGGTTGGAGCCTTAGCGGAGT 3′ | |
| (9) T_5R6E_HP | 5′ | 37 |
| | GCGAAACAAGAGGCTTGTGCTGCGTTTTC | |
| | GCAGCACTTTGAGGACTAGGGAGTT 3′ | |
| (10) T_5R8E_HP | 5′ | 38 |
| | CCAAATCATTACTTAGACGCTGGCTTTTG | |
| | CCAGCGTTTCCGGAACGTACCAAGC 3′ | |
| (11) T_5R10E_HP | 5′ | 39 |
| | AAAGATTCTAAATTGGCGACGGACTTTTG | |
| | TCCGTCGTTGCTTGAGATTCATTAC 3′ | |
| (12) T_5R12E_CYC_HP | 5′ | 40 |
| | CTCAGAGCGAGGCATAGGCTCCGCTTTTG | |
| | CGGAGCCTTGTAAGAGCACAGGTAG 3′ | |
| (13) T_3R2F_HP | 5′ | 41 |
| | TGTAGCATAACTTTCAGGCATCCGTTTTC | |
| | GGATGCCTTACAGTTTCTAATTGTA 3′ | |
| (14) T_3R4F_HP | 5′ | 42 |
| | TCGGTTTAGGTCGCTGGCTGACGCTTTTG | |
| | CGTCAGCTTAGGCTTGCAAAGACTT 3′ | |
| (15) T_3R6F_HP | 5′ | 43 |
| | TTTCATGATGACCCCCACCAGCCGTTTTC | |
| | GGCTGGTTTAGCGATTAAGGCGCAG 3′ | |
| (16) T_3R8F_HP | 5′ | 44 |
| | ACGGTCAATGACAAGACGGAGGCGTTTTC | |
| | GCCTCCGTTACCGGATATGGTTTAA 3′ | |
| (17) T_3R10F_HP | 5′ | 45 |
| | TTTCAACTACGGAACACTCGCTGCTTTTG | |
| | CAGCGAGTTACATTATTAACACTAT 3′ | |
| (18) T_3R12F_CYC_HP | 5′ | 46 |
| | CATAACCCACCGCCACCTGGCTCGTTTTC | |
| | GAGCCAGTTCCTCAGAAACAACGCC 3′ | |
| (19) T_3R2E_HP | 5′ | 47 |
| | TGCTAAACTCCACAGAGCCAGTGCTTTTG | |
| | CACTGGCTTCAGCCCTCTACCGCCA 3′ | |
| (20) T_3R4E_HP | 5′ | 48 |
| | ATATATTCTCAGCTTGCCGTCCGCTTTTG | |
| | CGGACGGTTCTTTCGAGTGGGATTT 3′ | |
| (21) T_3R6E_HP | 5′ | 49 |
| | CTCATCTTGGAAGTTTCGGATGGCTTTTG | |
| | CCATCCGTTCCATTAAACATAACCG 3′ | |
| (22) T_3R8E_HP | 5′ | 50 |
| | AGTAATCTTCATAAGGTCTGGTCGTTTTC | |
| | GACCAGATTGAACCGAACTAAAACA 3′ | |
| (23) T_3R10E_HP | 5′ | 51 |
| | ACGAACTATTAATCATGGCACCTGTTTTC | |
| | AGGTGCCTTTGTGAATTTCATCAAG 3′ | |
| (24) T_3R12E_CYC_HP | 5′ | 52 |
| | CCCTCAGATCGTTTACCGCTTGCGTTTTC | |
| | GCAAGCGTTCAGACGACTTAATAAA 3′ | |
| (25) T_1R2F_HP | 5′ | 53 |
| | CGTAACGAAAATGAATCCTGCCTGTTTTC | |
| | AGGCAGGTTTTTCTGTAGTGAATTT 3′ | |
| (26) T_1R4F_HP | 5′ | 54 |
| | CTTAAACAACAACCATCGGTGCCGTTTTC | |
| | GGCACCGTTCGCCCACGCGGGTAAA 3′ | |
| (27) T_1R6F_HP | 5′ | 55 |
| | ATACGTAAGAGGCAAACTCGGTCGTTTTC | |
| | GACCGAGTTAGAATACACTGACCAA 3′ | |
| (28) T_1R8F_HP | 5′ | 56 |
| | CTTTGAAAATAGGCTGCCGAGGACTTTTG | |
| | TCCTCGGTTGCTGACCTACCTTATG 3′ | |
| (29) T_1R1OF_HP | 5′ | 57 |
| | CGATTTTAGGAAGAAACGGCAGGCTTTTG | |
| | CCTGCCGTTAATCTACGGATAAAAA 3′ | |
| (30) T_1R12F_CYC_HP | 5′ | 58 |
| | CCAAAATATACTCAGGTGCGGTCGTTTTC | |
| | GACCGCATTAGGTTTAGATAGTTAG 3′ | |
| (31) T_1R2E_HP | 5′ | 59 |
| | ACGTTAGTTCTAAAGTCGCTTGGCTTTTG | |
| | CCAAGCGTTTTTGTCGTGATACAGG 3′ | |
| (32) T_1R4E_HP | 5′ | 60 |
| | CAATGACAGCTTGATATGGCGAGCTTTTG | |
| | CTCGCCATTCCGATAGTCTCCCTCA 3′ | |
| (33) T_1R6E_HP | 5′ | 61 |
| | AAACGAAATGCCACTACCACCTCGTTTTC | |
| | GAGGTGGTTCGAAGGCAGCCAGCAA 3′ | |
| (34) T_1R8E_HP | 5′ | 62 |
| | CCAGGCGCGAGGACAGCTCTGGACTTTTG | |
| | TCCAGAGTTATGAACGGGTAGAAAA 3′ | |
| (35) T_1R10E_HP | 5′ | 63 |
| | GGACGTTGAGAACTGGCGAGGCACTTTTG | |
| | TGCCTCGTTCTCATTATGCGCTAAT 3′ | |
| (36) T_1R12E_CYC_HP | 5′ | 64 |
| | TATCACCGGCGAGAGGCTGCGTCGTTTTC | |
| | GACGCAGTTCTTTTGCAATCCTGAA 3′ | |
| (37) T1R2F_HP | 5′ | 65 |
| | AGTGTACTATACATGGCTCCTGCGTTTTC | |
| | GCAGGAGTTCTTTTGATCTTTCCAG 3′ | |
| (38) T1R4F_HP | 5′ | 66 |
| | GAGCCGCCCCACCACCGTCAGGCGTTTTC | |
| | GCCTGACTTGGAACCGCTGCGCCGA 3′ | |
| (39) T1R6F_HP | 5′ | 67 |
| | AATCACCACCATTTGGCGTCCTGCTTTTG | |
| | CAGGACGTTGAATTAGACCAACCTA 3′ | |
| (40) T1R8F_HP | 5′ | 68 |
| | TACATACACAGTATGTCGGACCTGTTTTC | |
| | AGGTCCGTTTAGCAAACTGTACAGA 3′ | |
| (41) T1R10F_HP | 5′ | 69 |
| | ATCAGAGAGTCAGAGGCGAGGTCGTTTTC | |
| | GACCTCGTTGTAATTGAACCAGTCA 3′ | |
| (42) T1R12F_CYC_HP | 5′ | 70 |
| | TCTTACCATATAAGTACCGAGGCGTTTTC | |
| | GCCTCGGTTTAGCCCGGAATAGGTG 3′ | |
| (43) T1R2E_HP | 5′ | 71 |
| | TAAGCGTCGGTAATAACAGGAGCGTTTTC | |
| | GCTCCTGTTGTTTTAACCCGTCGAG 3′ | |
| (44) T1R4E_HP | 5′ | 72 |
| | AACCAGAGACCCTCAGGCAGTCGCTTTTG | |
| | CGACTGCTTAACCGCCACGTTCCAG 3′ | |
| (45) T1R6E_HP | 5′ | 73 |
| | GACTTGAGGTAGCACCGTCTGGCGTTTTC | |
| | GCCAGACTTATTACCATATCACCGG 3′ | |
| (46) T1R8E_HP | 5′ | 74 |
| | TTATTACGTAAAGGTGTGGCTGCGTTTTC | |
| | GCAGCCATTGCAACATACCGTCACC 3′ | |
| (47) T1R10E_HP | 5′ | 75 |
| | TGAACAAAGATAACCCAGTGCCTGTTTTC | |
| | AGGCACTTTACAAGAATAAGACTCC 3′ | |
| (48) T1R12E_CYC_HP | 5′ | 76 |
| | AGGGTTGAACGCTAACGCCAGGACTTTTG | |
| | TCCTGGCTTGAGCGTCTGAACACCC 3′ | |
| (49) T3R2F_HP | 5′ | 77 |
| | TGCCTTGACAGTCTCTGTCGGTGCTTTTG | |
| | CACCGACTTGAATTTACCCCTCAGA 3′ | |
| (50) T3R4F_HP | 5′ | 78 |
| | GCCACCACTCTTTTCACGGTCGGCTTTTG | |
| | CCGACCGTTTAATCAAATAGCAAGG 3′ | |
| (51) T3R6F_HP | 5′ | 79 |
| | CCGGAAACTAAAGGTGGACCTGGCTTTTG | |
| | CCAGGTCTTAATTATCATAAAAGAA 3′ | |
| (52) T3R8F_HP | 5′ | 80 |
| | ACGCAAAGAAGAACTGTCGGCTCGTTTTC | |
| | GAGCCGATTGCATGATTTGAGTTAA 3′ | |
| (53) T3R10F_HP | 5′ | 81 |
| | GCCCAATAGACGGGAGCACAGGCGTTTTC | |
| | GCCTGTGTTAATTAACTTTCCAGAG 3′ | |
| (54) T3R12F_CYC_HP | 5′ | 82 |
| | CCTAATTTACCAGGCGTCGGAGCGTTTTC | |
| | GCTCCGATTGATAAGTGGGGGTCAG 3′ | |
| (55) T3R2E_HP | 5′ | 83 |
| | GGAAAGCGGTAACAGTGTGGCAGCTTTTG | |
| | CTGCCACTTGCCCGTATCGGGGTTT 3′ | |
| (56) T3R4E_HP | 5′ | 84 |
| | GTTTGCCACCTCAGAGACCAGGCGTTTTC | |
| | GCCTGGTTTCCGCCACCGCCAGAAT 3′ | |
| (57) T3R6E_HP | 5′ | 85 |
| | TTATTCATGTCACCAAGCTCGCTGTTTTC | |
| | AGCGAGCTTTGAAACCATTATTAGC 3′ | |
| (58) T3R8E_HP | 5′ | 86 |
| | ATACCCAAACACCACGCCTACCGCTTTTG | |
| | CGGTAGGTTGAATAAGTGACGGAAA 3′ | |
| (59) T3R10E_HP | 5′ | 87 |
| | GCGCATTAATAAGAGCCTGGACGCTTTTG | |
| | CGTCCAGTTAAGAAACAATAACGGA 3′ | |
| (60) T3R12E_CYC_HP | 5′ | 88 |
| | TGCTCAGTGCCAGTTAGGTGGTCGTTTTC | |
| | GACCACCTTCAAAATAAACAGGGAA 3′ | |
| (61) T5R2F_HP | 5′ | 89 |
| | AATGCCCCATAAATCCGCTCGGACTTTTG | |
| | TCCGAGCTTTCATTAAAAGAACCAC 3′ | |
| (62) T5R4F_HP | 5′ | 90 |
| | CACCAGAGTTCGGTCAGCCGAGCGTTTTC | |
| | GCTCGGCTTTAGCCCCCTCGATAGC 3′ | |
| (63) T5R6F_HP | 5′ | 91 |
| | AGCACCGTAGGGAAGGTCGGAGGCTTTTG | |
| | CCTCCGATTTAAATATTTTATTTTG 3′ | |
| (64) T5R8F_HP | 5′ | 92 |
| | TCACAATCCCGAGGAACTGGTGGCTTTTG | |
| | CCACCAGTTACGCAATAATGAAATA 3′ | |
| (65) T5R10F_HP | 5′ | 93 |
| | GCAATAGCAGAGAATACCGCAGGCTTTTG | |
| | CCTGCGGTTACATAAAAACAGCCAT 3′ | |
| (66) T5R12F_CYC_HP | 5′ | 94 |
| | ATTATTTAGAAGGATTGCCATCGCTTTTG | |
| | CGATGGCTTAGGATTAGAAACAGTT 3′ | |
| (67) T5R2E_HP | 5′ | 95 |
| | ACAAACAACTGCCTATCACGACGCTTTTG | |
| | CGTCGTGTTTTCGGAACCTGAGACT 3′ | |
| (68) T5R4E_HP | 5′ | 96 |
| | TCGGCATTCCGCCGCCGTCGCTGCTTTTG | |
| | CAGCGACTTAGCATTGATGATATTC 3′ | |
| (69) T5R6E_HP | 5′ | 97 |
| | ATTGAGGGAATCAGTACGGAGCACTTTTG | |
| | TGCTCCGTTGCGACAGACGTTTTCA 3′ | |
| (70) T5R8E_HP | 5′ | 98 |
| | GAAGGAAAAATAGAAAGCCTAGCGTTTTC | |
| | GCTAGGCTTATTCATATTTCAACCG 3′ | |
| (71) T5R10E_HP | 5′ | 99 |
| | CTTTACAGTATCTTACCGCTCGTGTTTTC | |
| | AACGGCGTTCGAAGCCCAGTTACCA 3′ | |
| (72) T5R12E_CYC_HP | 5′ | 100 |
| | CCTCAAGATCCCAATCCGTGGAGCTTTTG | |
| | CTCCACGTTCAAATAAGATAGCAGC 3′ | |

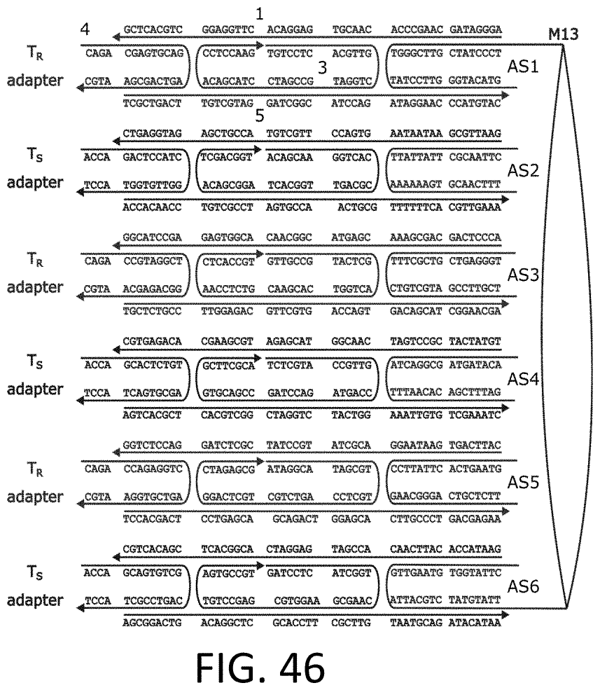

As shown in FIG. 46, there are 6 tile adapters around the circumference of the seed face (AS1-AS6), 3 adapters that present the T_{R} sticky ends and 3 adapters that present the T_{S} sticky ends. The adapters resemble DNA tiles composed but are bound to M13 DNA (gray) where strand 2 would normally be on the tiles. Sequences of DNA origami seed tile adapters are shown in Table 5. All strands were ordered from Integrated DNA Technologies, Inc (TDT), strands 1, 3, and 5 were all ordered unpurified and all sticky end strands (strand 4) were ordered PAGE purified.

TABLE 5
Sequences.

| | | SEQ |
|---|---|---|
| Strand | | ID |
| name | Sequence | NO: |
| T_{R} | AGGGATAGCAAGCCCACAACGTGAGG | 101 |
| adapter | ACACTTGGAGGCTGCACTCG | |
| AS1-1 | | |
| T_{R} | TGTCCTCACGTTGCTGGATGCCGATC | 102 |
| adapter | CTACGACACCTCCAAG | |
| AS1-3 | | |
| T_{R} | TCGCTGACTTGTCGTAGGATCGGCAT | 103 |
| adapter | CCAGATAGGAACCCATGTAC | |
| AS1-5 | | |
| T_{R} | CAGACGAGTGCAGAGTCAGCGAATGC | 104 |
| adapter | | |
| AS1-4 | | |
| T_{S} | GAATTGCGAATAATAAGTGACCTTGC | 105 |
| adapter | TGTACCGTCGAGATGGAGTC | |
| AS2-1 | | |
| T_{S} | ACAGCAAGGTCACCGCAGTTGGCACT | 106 |
| adapter | AGGCGACATCGACGGT | |
| AS2-3 | | |
| T_{S} | ACCACAACCTGTCGCCTAGTGCCAAC | 107 |
| adapter | TGCGTTTTTTCACGTTGAAA | |
| AS2-5 | | |
| T_{S} | ACCAGACTCCATCGGTTGTGGTACCT | 108 |
| adapter | | |
| AS2-4 | | |
| T_{R} | ACCCTCAGCAGCGAAACGAGTACGGC | 109 |
| adapter | AACACGGTGAGAGCCTACGG | |
| AS3-1 | | |
| T_{R} | GTTGCCGTACTCGACTGGTCACGAAC | 110 |
| adapter | GTCTCCAACTCACCGT | |
| AS3-3 | | |
| T_{R} | TGCTCTGCCTTGGAGACGTTCGTGAC | 111 |
| adapter | CAGTGACAGCATCGGAACGA | |
| AS3-5 | | |
| T_{R} | CAGACCGTAGGCTGGCAGAGCAATGC | 112 |
| adapter | | |
| AS3-4 | | |
| T_{S} | TGTATCATCGCCTGATCAACGGTACG | 113 |
| adapter | AGATGCGAAGCACAGAGTGC | |
| AS4-1 | | |
| T_{S} | TCTCGTACCGTTGCCAGTAGACCTAG | 114 |
| adapter | CCGACGTGGCTTCGCA | |
| AS4-3 | | |
| T_{S} | AGTCACGCTCACGTCGGCTAGGTCTA | 115 |
| adapter | CTGGAAATTGTGTCGAAATC | |
| AS4-5 | | |
| T_{S} | ACCAGCACTCTGTAGCGTGACTACCT | 116 |
| adapter | | |
| AS4-4 | | |
| T_{R} | CATTCAGTGAATAAGGACGCTATGCC | 117 |
| adapter | TATCGCTCTAGGACCTCTGG | |
| AS5-1 | | |
| T_{R} | ATAGGCATAGCGTTGCTCCAGTCTGC | 118 |
| adapter | TGCTCAGGCTAGAGCG | |
| AS5-3 | | |
| T_{R} | TCCACGACTCCTGAGCAGCAGACTGG | 119 |
| adapter | AGCACTTGCCCTGACGAGAA | |
| AS5-5 | | |
| T_{R} | CAGACCAGAGGTCAGTCGTGGAATGC | 120 |
| adapter | | |
| AS5-4 | | |
| T_{S} | GAATACCACATTCAACACCGATGAGG | 121 |
| adapter | ATCACGGCACTCGACACTGC | |
| AS6-1 | | |
| T_{S} | GATCCTCATCGGTCAAGCGAAGGTGC | 122 |
| adapter | GAGCCTGTAGTGCCGT | |
| AS6-3 | | |
| T_{S} | AGCGGACTGACAGGCTCGCACCTTCG | 123 |
| adapter | CTTGTAATGCAGATACATAA | |
| AS6-5 | | |
| T_{S} | ACCAGCAGTGTCGCAGTCCGCTACCT | 124 |
| adapter | | |
| AS6-4 | | |

DNA origami terminus strands for fluorescent labeling are shown below in Table 6. All labeling strands ordered unpurified from IDT. Fluorescent strands were ordered HPLC purified from TIDT.

TABLE 6
Sequences.

| | | SEQ |
|---|---|---|
| | | ID |
| Strand Name | Sequence | NO: |
| Fluorescent | | |
| strands | | |
| atto647 strand | /5ATTO647NN/AAGCGTAGTCGGATCTC 3′ | 125 |
| (S2 seeds) | | |
| atto488 strand | /5ATTO488N/AAGCGTAGTCGGATCTC 3′ | 126 |
| (S1 seeds) | | |
| Labeling strands | | |
| Unused_m13mp18_01 | 5′ AAATTCTTACCAGTATAAAGCCAACTTTTGAGATCCGACTACGC 3′ | 127 |
| Unused_m13mp18_02 | 5′ GCCTGTTTAGTATCATATGCGTTATTTTTGAGATCCGACTACGC 3′ | 128 |
| Unused_m13mp18_03 | 5′ ACACCGGAATCATAATTACTAGAAATTTTGAGATCCGACTACGC 3′ | 129 |
| Unused_m13mp18_04 | 5′ GATAAATAAGGCGTTAAATAAGAATTTTTGAGATCCGACTACGC 3′ | 130 |
| Unused_m13mp18_05 | 5′ TTTAATGGTTTGAAATACCGACCGTTTTTGAGATCCGACTACGC 3′ | 131 |
| Unused_m13mp18_06 | 5′ TTAGTTAATTTCATCTTCTGACCTATTTTGAGATCCGACTACGC 3′ | 132 |
| Unused_m13mp18_07 | 5′ ACGCGAGAAAACTTTTTCAAATATATTTTGAGATCCGACTACGC 3′ | 133 |
| Unused_m13mp18_08 | 5′ GATGCAAATCCAATCGCAAGACAAATTTTGAGATCCGACTACGC 3′ | 134 |
| Unused_m13mp18_09 | 5′ TGGGTTATATAACTATATGTAAATGTTTTGAGATCCGACTACGC 3′ | 135 |
| Unused_m13mp18_10 | 5′ ACTACCTTTTTAACCTCCGGCTTAGTTTTGAGATCCGACTACGC 3′ | 136 |
| Unused_m13mp18_11 | 5′ AATTTATCAAAATCATAGGTCTGAGTTTTGAGATCCGACTACGC 3′ | 137 |
| Unused_m13mp18_12 | 5′ TTAAGACGCTGAGAAGAGTCAATAGTTTTGAGATCCGACTACGC 3′ | 138 |
| Unused_m13mp18_13 | 5′ TCCTTGAAAACATAGCGATAGCTTATTTTGAGATCCGACTACGC 3′ | 139 |
| Unused_m13mp18_14 | 5′ TCGCTATTAATTAATTTTCCCTTAGTTTTGAGATCCGACTACGC 3′ | 140 |
| Unused_m13mp18_15 | 5′ AGTGAATAACCTTGCTTCTGTAAATTTTTGAGATCCGACTACGC 3′ | 141 |
| Unused_m13mp18_16 | 5′ GAAACAGTACATAAATCAATATATGTTTTGAGATCCGACTACGC 3′ | 142 |
| Unused_m13mp18_17 | 5′ ATTTCATTTGAATTACCTTTTTTAATTTTGAGATCCGACTACGC 3′ | 143 |
| Unused_m13mp18_18 | 5′ AGAAAACAAAATTAATTACATTTAATTTTGAGATCCGACTACGC 3′ | 144 |
| Unused_m13mp18_19 | 5′ CAAAAGAAGATGATGAAACAAACATTTTTGAGATCCGACTACGC 3′ | 145 |
| Unused_m13mp18_20 | 5′ GCGAATTATTCATTTCAATTACCTGTTTTGAGATCCGACTACGC 3′ | 146 |
| Unused_m13mp18_21 | 5′ AATACCAAGTTACAAAATCGCGCAGTTTTGAGATCCGACTACGC 3′ | 147 |
| Unused_m13mp18_22 | 5′ CAATAACGGATTCGCCTGATTGCTTTTTTGAGATCCGACTACGC 3′ | 148 |
| Unused_m13mp18_23 | 5′ TAACAGTACCTTTTACATCGGGAGATTTTGAGATCCGACTACGC 3′ | 149 |
| Unused_m13mp18_24 | 5′ CAGGTTTAACGTCAGATGAATATACTTTTGAGATCCGACTACGC 3′ | 150 |
| Unused_m13mp18_25 | 5′ CAGAAATAAAGAAATTGCGTAGATTTTTTGAGATCCGACTACGC 3′ | 151 |
| Unused_m13mp18_26 | 5′ CCATATCAAAATTATTTGCACGTAATTTTGAGATCCGACTACGC 3′ | 152 |
| Unused_m13mp18_27 | 5′ TCTGAATAATGGAAGGGTTAGAACCTTTTGAGATCCGACTACGC 3′ | 153 |
| Unused_m13mp18_28 | 5′ TATAATCCTGATTGTTTGGATTATATTTTGAGATCCGACTACGC 3′ | 154 |
| Unused_m13mp18_29 | 5′ GATTATCAGATGATGGCAATTCATCTTTTGAGATCCGACTACGC 3′ | 155 |
| Unused_m13mp18_30 | 5′ AAGGAGCGGAATTATCATCATATTCTTTTGAGATCCGACTACGC 3′ | 156 |
| Unused_m13mp18_31 | 5′ CATTTTGCGGAACAAAGAAACCACCTTTTGAGATCCGACTACGC 3′ | 157 |
| Unused_m13mp18_32 | 5′ TAATTTTAAAAGTTTGAGTAACATTTTTTGAGATCCGACTACGC 3′ | 158 |
| Unused_m13mp18_33 | 5′ GTATTAAATCCTTTGCCCGAACGTTTTTTGAGATCCGACTACGC 3′ | 159 |
| Unused_m13mp18_34 | 5′ TAGACTTTACAAACAATTCGACAACTTTTGAGATCCGACTACGC 3′ | 160 |
| Unused_m13mp18_35 | 5′ ATAATACATTTGAGGATTTAGAAGTTTTTGAGATCCGACTACGC 3′ | 161 |
| Unused_m13mp18_36 | 5′ CAACTAATAGATTAGAGCCGTCAATTTTTGAGATCCGACTACGC 3′ | 162 |
| Unused_m13mp18_37 | 5′ TATCTAAAATATCTTTAGGAGCACTTTTTGAGATCCGACTACGC 3′ | 163 |
| Unused_m13mp18_38 | 5′ ACTGATAGCCCTAAAACATCGCCATTTTTGAGATCCGACTACGC 3′ | 164 |
| Unused_m13mp18_39 | 5′ GAATGGCTATTAGTCTTTAATGCGCTTTTGAGATCCGACTACGC 3′ | 165 |
| Unused_m13mp18_40 | 5′ AGAATACGTGGCACAGACAATATTTTTTTGAGATCCGACTACGC 3′ | 166 |
| Unused_m13mp18_41 | 5′ ATAGAACCCTTCTGACCTGAAAGCGTTTTGAGATCCGACTACGC 3′ | 167 |
| Unused_m13mp18_42 | 5′ ATAAAAGGGACATTCTGGCCAACAGTTTTGAGATCCGACTACGC 3′ | 168 |
| Unused_m13mp18_43 | 5′ GCAGATTCACCAGTCACACGACCAGTTTTGAGATCCGACTACGC 3′ | 169 |
| Unused_m13mp18_44 | 5′ ATCGTCTGAAATGGATTATTTACATTTTTGAGATCCGACTACGC 3′ | 170 |
| Unused_m13mp18_45 | 5′ ATGGAAATACCTACATTTTGACGCTTTTTGAGATCCGACTACGC 3′ | 171 |
| Unused_m13mp18_46 | 5′ CCAGCCATTGCAACAGGAAAAACGCTTTTGAGATCCGACTACGC 3′ | 172 |
| Unused_m13mp18_47 | 5′ CTGGTAATATCCAGAACAATATTACTTTTGAGATCCGACTACGC 3′ | 173 |
| Unused_m13mp18_48 | 5′ GTAGAAGAACTCAAACTATCGGCCTTTTTGAGATCCGACTACGC 3′ | 174 |
| Unused_m13mp18_49 | 5′ TGATTAGTAATAACATCACTTGCCTTTTTGAGATCCGACTACGC 3′ | 175 |
| Unused_m13mp18_50 | 5′ AAATTAACCGTTGTAGCAATACTTCTTTTGAGATCCGACTACGC 3′ | 176 |
| Unused_m13mp18_51 | 5′ CCGAGTAAAAGAGTCTGTCCATCACTTTTGAGATCCGACTACGC 3′ | 177 |
| Unused_m13mp18_52 | 5′ GAAGTGTTTTTATAATCAGTGAGGCTTTTGAGATCCGACTACGC 3′ | 178 |
| Unused_m13mp18_53 | 5′ GACAGGAACGGTACGCCAGAATCCTTTTTGAGATCCGACTACGC 3′ | 179 |
| Unused_m13mp18_54 | 5′ AACAGGAGGCCGATTAAAGGGATTTTTTTGAGATCCGACTACGC 3′ | 180 |
| Unused_m13mp18_55 | 5′ TCCTCGTTAGAATCAGAGCGGGAGCTTTTGAGATCCGACTACGC 3′ | 181 |
| Unused_m13mp18_56 | 5′ GCTTTGACGAGCACGTATAACGTGCTTTTGAGATCCGACTACGC 3′ | 182 |
| Unused_m13mp18_57 | 5′ CGCCGCTACAGGGCGCGTACTATGGTTTTGAGATCCGACTACGC 3′ | 183 |
| Unused_m13mp18_58 | 5′ TAACCACCACACCCGCCGCGCTTAATTTTGAGATCCGACTACGC 3′ | 184 |
| Unused_m13mp18_59 | 5′ TGGCAAGTGTAGCGGTCACGCTGCGTTTTGAGATCCGACTACGC 3′ | 185 |
| Unused_m13mp18_60 | 5′ AAGCGAAAGGAGCGGGCGCTAGGGCTTTTGAGATCCGACTACGC 3′ | 186 |
| Unused_m13mp18_61 | 5′ CGAACGTGGCGAGAAAGGAAGGGAATTTTGAGATCCGACTACGC 3′ | 187 |
| Unused_m13mp18_62 | 5′ GATTTAGAGCTTGACGGGGAAAGCCTTTTGAGATCCGACTACGC 3′ | 188 |
| Unused_m13mp18_63 | 5′ TAAATCGGAACCCTAAAGGGAGCCCTTTTGAGATCCGACTACGC 3′ | 189 |
| Unused_m13mp18_64 | 5′ TTTTGGGGTCGAGGTGCCGTAAAGCTTTTGAGATCCGACTACGC 3′ | 190 |
| Unused_m13mp18_65 | 5′ TACGTGAACCATCACCCAAATCAAGTTTTGAGATCCGACTACGC 3′ | 191 |
| Unused_m13mp18_66 | 5′ AAACCGTCTATCAGGGCGATGGCCCTTTTGAGATCCGACTACGC 3′ | 192 |
| Unused_m13mp18_67 | 5′ ACGTGGACTCCAACGTCAAAGGGCGTTTTGAGATCCGACTACGC 3′ | 193 |
| Unused_m13mp18_68 | 5′ TTTGGAACAAGAGTCCACTATTAAATTTTGAGATCCGACTACGC 3′ | 194 |
| Unused_m13mp18_69 | 5′ CCGAGATAGGGTTGAGTGTTGTTCCTTTTGAGATCCGACTACGC 3′ | 195 |
| Unused_m13mp18_70 | 5′ AAATCCCTTATAAATCAAAAGAATATTTTGAGATCCGACTACGC 3′ | 196 |
| Unused_m13mp18_71 | 5′ TGTTTGATGGTGGTTCCGAAATCGGTTTTGAGATCCGACTACGC 3′ | 197 |
| Unused_m13mp18_72 | 5′ CTGGTTTGCCCCAGCAGGCGAAAATTTTTGAGATCCGACTACGC 3′ | 198 |
| Unused_m13mp18_73 | 5′ TGAGAGAGTTGCAGCAAGCGGTCCATTTTGAGATCCGACTACGC 3′ | 199 |
| Unused_m13mp18_74 | 5′ AGCTGATTGCCCTTCACCGCCTGGCTTTTGAGATCCGACTACGC 3′ | 200 |
| Unused_m13mp18_75 | 5′ TTTCTTTTCACCAGTGAGACGGGCATTTTGAGATCCGACTACGC 3′ | 201 |
| Unused_m13mp18_76 | 5′ GTTTGCGTATTGGGCGCCAGGGTGGTTTTGAGATCCGACTACGC 3′ | 202 |
| Unused_m13mp18_77 | 5′ GAATCGGCCAACGCGCGGGGAGAGGTTTTGAGATCCGACTACGC 3′ | 203 |
| Unused_m13mp18_78 | 5′ GAAACCTGTCGTGCCAGCTGCATTATTTTGAGATCCGACTACGC 3′ | 204 |
| Unused_m13mp18_79 | 5′ TGCGCTCACTGCCCGCTTTCCAGTCTTTTGAGATCCGACTACGC 3′ | 205 |
| Unused_m13mp18_80 | 5′ GAGTGAGCTAACTCACATTAATTGCTTTTGAGATCCGACTACGC 3′ | 206 |
| Unused_m13mp18_81 | 5′ TAAAGTGTAAAGCCTGGGGTGCCTATTTTGAGATCCGACTACGC 3′ | 207 |
| Unused_m13mp18_82 | 5′ TTCCACACAACATACGAGCCGGAAGTTTTGAGATCCGACTACGC 3′ | 208 |
| Unused_m13mp18_83 | 5′ CTGTGTGAAATTGTTATCCGCTCACTTTTGAGATCCGACTACGC 3′ | 209 |
| Unused_m13mp18_84 | 5′ ATTCGTAATCATGGTCATAGCTGTTTTTTGAGATCCGACTACGC 3′ | 210 |
| Unused_m13mp18_85 | 5′ TAGAGGATCCCCGGGTACCGAGCTCTTTTGAGATCCGACTACGC 3′ | 211 |
| Unused_m13mp18_86 | 5′ CAAGCTTGCATGCCTGCAGGTCGACTTTTGAGATCCGACTACGC 3′ | 212 |
| Unused_m13mp18_87 | 5′ ACGACGTTGTAAAACGACGGCCAGTTTTTGAGATCCGACTACGC 3′ | 213 |
| Unused_m13mp18_88 | 5′ TTGGGTAACGCCAGGGTTTTCCCAGTTTTGAGATCCGACTACGC 3′ | 214 |
| Unused_m13mp18_89 | 5′ AGGGGGATGTGCTGCAAGGCGATTATTTTGAGATCCGACTACGC 3′ | 215 |
| Unused_m13mp18_90 | 5′ CTCTTCGCTATTACGCCAGCTGGCGTTTTGAGATCCGACTACGC 3′ | 216 |
| Unused_m13mp18_91 | 5′ CTGTTGGGAAGGGCGATCGGTGCGGTTTTGAGATCCGACTACGC 3′ | 217 |
| Unused_m13mp18_92 | 5′ GCGCCATTCGCCATTCAGGCTGCGCTTTTGAGATCCGACTACGC 3′ | 218 |
| Unused_m13mp18_93 | 5′ CGCTTCTGGTGCCGGAAACCAGGCATTTTGAGATCCGACTACGC 3′ | 219 |
| Unused_m13mp18_94 | 5′ ATCGCACTCCAGCCAGCTTTCCGGCTTTTGAGATCCGACTACGC 3′ | 220 |
| Unused_m13mp18_95 | 5′ GACGACGACAGTATCGGCCTCAGGATTTTGAGATCCGACTACGC 3′ | 221 |
| Unused_m13mp18_96 | 5′ GTAACCGTGCATCTGCCAGTTTGAGTTTTGAGATCCGACTACGC 3′ | 222 |
| Unused_m13mp18_97 | 5′ GGTCACGTTGGTGTAGATGGGCGCATTTTGAGATCCGACTACGC 3′ | 223 |
| Unused_m13mp18_98 | 5′ AAACGGCGGATTGACCGTAATGGGATTTTGAGATCCGACTACGC 3′ | 224 |
| Unused_m13mp18_99 | 5′ ACAACCCGTCGGATTCTCCGTGGGATTTTGAGATCCGACTACGC 3′ | 225 |
| Unused_m13mp18_100 | 5′ TTCATCAACATTAAATGTGAGCGAGTTTTGAGATCCGACTACGC 3′ | 226 |

### Example 18

To model seeded nanotube growth a stochastic kinetic model of tile attachment/detachment was used. The Gillespie algorithm was used to simulate nanotube growth beginning from a stable nucleating facet for a tube six tiles in circumference based on the design of the seed. A 150 nM tile concentration was used in all the simulations. For the seed concentrations in the simulations, 75% of the concentrations used in these experiments (e.g., 75% of 0.1 nM, 0.33 nM, or 1 nM). This adjustment was made because it was observed that 20-30% of the DNA origami seeds failed to nucleate nanotubes even in conditions where the tile concentration is above the nucleation barrier for growth. This is presumably due to some of the DNA origami seeds being incorrectly folded or possessing incomplete or incorrect adapters. These incorrect seeds would likely have much higher barriers to nucleation than the correct seeds and thus they may fail to nucleate growth under the reaction conditions. To capture this effect in the model, it was assumed that 75% of the seeds were capable of nucleating nanotubes and reduced the seed concentrations in the simulation by 25% compared to the concentrations used in the experiments. To compare the simulation results to the experimental results, the simulated fractions of seeds nucleating nanotubes were calculated based on the total experimental seed concentrations (e.g., 100% of seeds nucleating nanotubes in the simulations is about 75% of seeds nucleating nanotubes in these experiments).

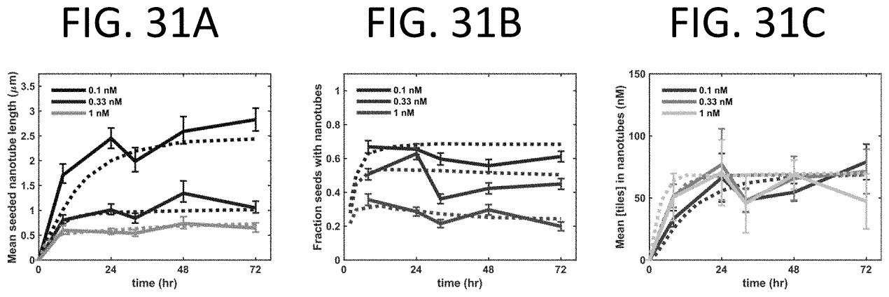

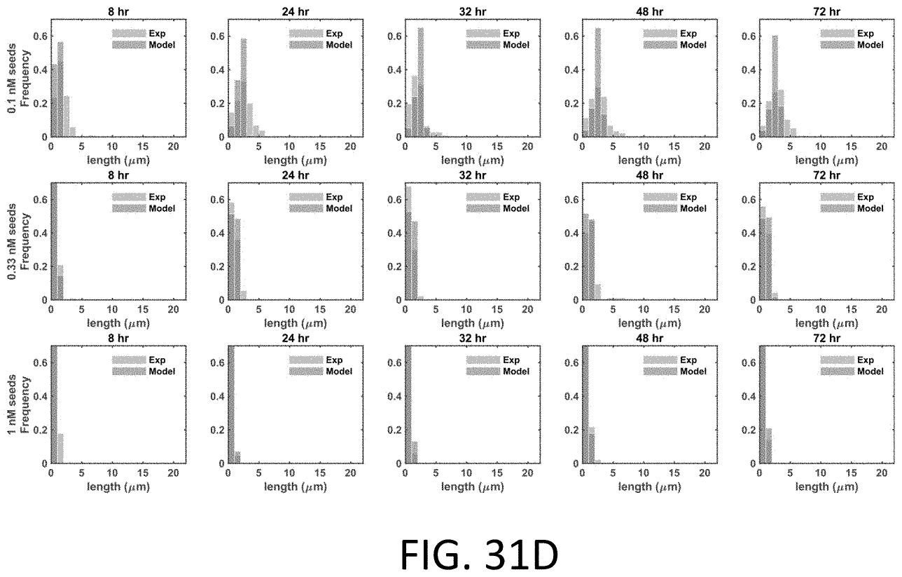

In the seeded nanotube growth simulations, there were three model parameters that were varied to recapitulate the experimental data: the rate that tiles can attach to a seed and/or a growing nanotube face (k_{ON}), the free energy of interaction between a tile and the growth face of a nanotube (ΔG_{T-NT}), and the nucleation barrier for growth from a seed (k_{OFF, T-S}). ΔG_{T-NT} determines the detachment rate of a tile from the end of a nanotube through the relationship k_{OFF T-NT}=k_{ON}*exp(ΔG_{T-NT}/RT), where R is the universal gas constant and T is the absolute temperature. A nucleation barrier for growth from an origami seed has been previously described and was modeled here by assuming the off rate from the first position on the seed was higher than the off rate from all other positions (k_{OFF, T-S}>k_{OFF, T-NT}). It was found that the values: k_{ON}=2×10⁵ M⁻¹s⁻¹, ΔG_{T-NT}=−9.3 kcal/mol, and k_{OFF, T-S}=6*k_{OFF, T-NT} best recapitulated the experimental data (FIG. 31). Unless otherwise stated, these parameters were used in all subsequent modeling of nanotube growth kinetics in this study.

### Example 19

Results in FIG. 24 (see also FIG. 31) show that batch nanotube growth with 150 nM tiles resulted in roughly 65-70% of the seeds nucleating growth at the lowest seed concentration tested. Initially, it was assumed this was just the inherent yield of the seeds for the growth conditions selected (e.g., it is possible to nucleate nearly 100% of the seeds but under these batch growth conditions maximum possible nucleation yield was not obtained). The initial simulations of nanotube growth with tile concentration buffering (FIG. 26) predicted that buffering should increase the percentage of seeds nucleating nanotubes to 100% for all of the seed concentrations tested experimentally; however, these experiments resulted in about 70-80% of the seeds nucleating nanotubes across all the seed concentrations (FIG. 27).

As shown in FIG. 28, growth was initiated from two different seeds at different times during the growth process. In the experiment, seeds with one fluorescent dye (S1) were initially incubated with the tile concentration buffering components for 24 hours and roughly 75-80% of the S1 seeds nucleated nanotubes. After 24 hours of growth, seeds identical to the S1 seeds but labeled with a different fluorescent dye (S2) were added and by the end of the experiment 75-80% of the S2 seeds nucleated nanotubes. These results indicate that the free tile concentration when only the S1 seeds are present is still above the nucleation barrier for seeded growth because nanotubes begin to nucleate from the S2 seeds when these new seeds are added. Thus, the 20-25% of S1 seeds that did not nucleate in the first 24 hours likely had a higher barrier to nucleation than the S1 seeds that nucleated growth in the first 24 hours and the S2 seeds that nucleated growth when added after 24 hours. These results suggest that the maximum percentage of seeds that can nucleate nanotubes in these systems is roughly 70-80%.

### Example 20

To model the nanotube growth process with tile concentration buffering, the nanotube growth parameters obtained from the stochastic kinetic model of nanotube growth were used with 150 nM tiles (ΔG=−9.3 kcal/mol and k_{OFF, T-S}=6*k_{OFF, T-NT}) and assumed reaction rate constants for the forward and reverse rates of the tile buffering reactions based on literature values for toehold mediated strand displacement reactions of the same toehold lengths. Given the Source and Initiator complexes react via 2-base toehold mediated stand displacement it was initially assumed the buffering forward reaction rate (k_{f}) to be 1×10² M⁻¹s⁻¹. Likewise, the buffering reverse reaction rate (kᵣ) was set to be 1×104 M⁻¹s⁻¹ since the Sink strands and active tiles react via a 4-base toehold mediated strand displacement process. It was assumed that the buffering reaction rates for the T_{R} and T_{S} tiles were the same.

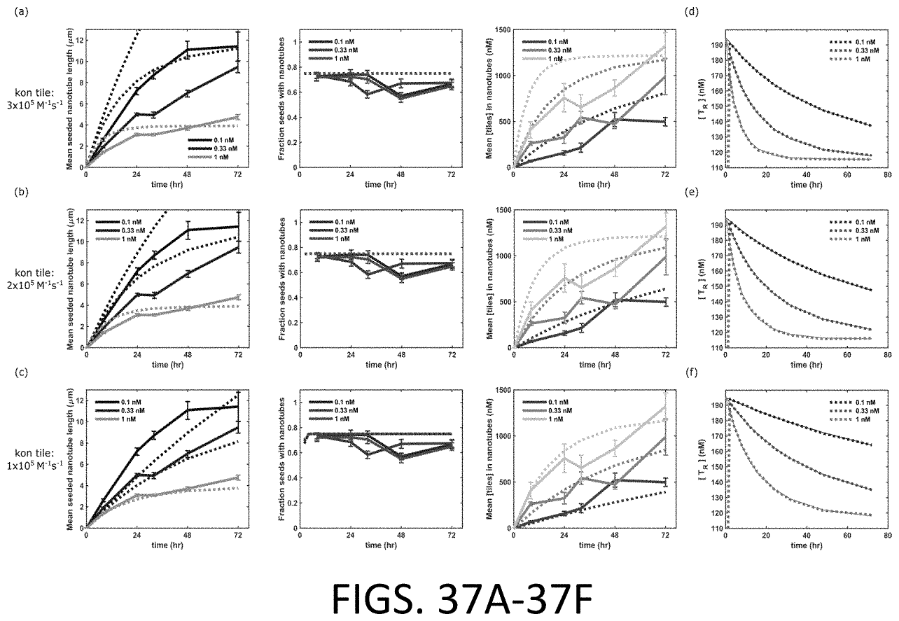

With these parameters nanotube growth was simulated with buffering using three different values for the rate of tile attachment (k_{ON}). With high k_{ON} values the simulations predicted rapid growth rates that began to slow down before 24 hours for the higher seed concentrations. The simulated growth kinetics were different than the experimental growth kinetics which were much more linear over the 72 hours growth period (FIGS. 37A-37B). The lowest k_{ON} value used in the simulations resulted in agreement with the experimental growth kinetics for the high seed concentrations but predicted much slower growth than observed for the lowest seed concentration (FIG. 37C).

The rate constants assumed for the forward and reverse buffering reactions were large enough so that the buffer should be able to keep up with nanotube growth (e.g., the buffer should be able to replace free tiles in the reaction faster than nanotube growth can deplete them) and thus the free tile concentration in the reaction should always be at its equilibrium value. To evaluate whether this was the case, the simulated concentrations of the tile buffering components (Sᵢ, Iᵢ, Tᵢ, and Nᵢ) were used and conducted an equilibrium analysis at each timepoint in the simulation. The concentration of the free tiles was plotted from both the stochastic simulations and the equilibrium analysis and it was found that these two concentrations were the same over the whole simulation time (FIG. 37), indicating that tile buffering was faster than nanotube growth for this set of buffering rate constants.

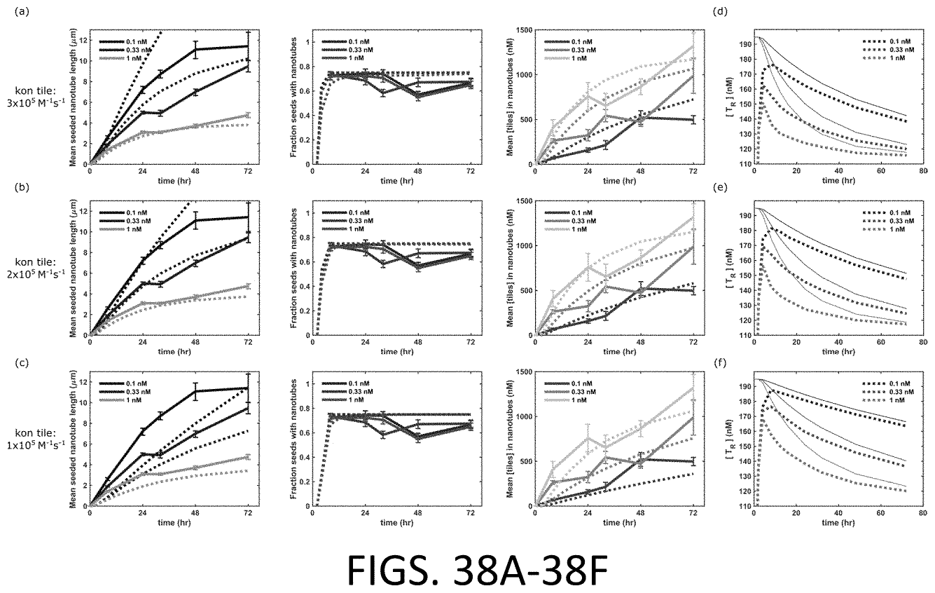

Since the simulations seemed to be overpredicting the initial nanotube growth rate compared to the experiments, it was thought that the assumed tile buffering reaction rate constants (k_{f} and kᵣ) were too high. Thus, both of the tile buffering reaction rate constants were lowered two orders of magnitude (k_{f}=1×10⁰ M⁻¹s⁻¹ and kᵣ=1×10² M⁻¹s⁻¹) and the simulations were conducted again. This set of tile buffering reaction rates produced fairly linear nanotube growth over the course of 72 hrs and for a k_{ON} of 2×10⁵ M⁻¹s⁻¹ (the value used for the simulated growth of seeded nanotubes with 150 nM tiles) agreement was established with the experimental results (FIG. 38). Conducting an equilibrium analysis on the simulated concentrations of the tile buffering components (Sᵢ, Iᵢ, Tᵢ, and Nᵢ) during these simulations it was found that, for the new tile buffering k_{f} and kᵣ values used, the buffer cannot keep up with nanotube growth (FIG. 38). Additionally, the higher the seed concentration, the further the simulated free tile concentrations are from their equilibrium values, resulting in the different initial growth rates seem across the different seed concentrations.

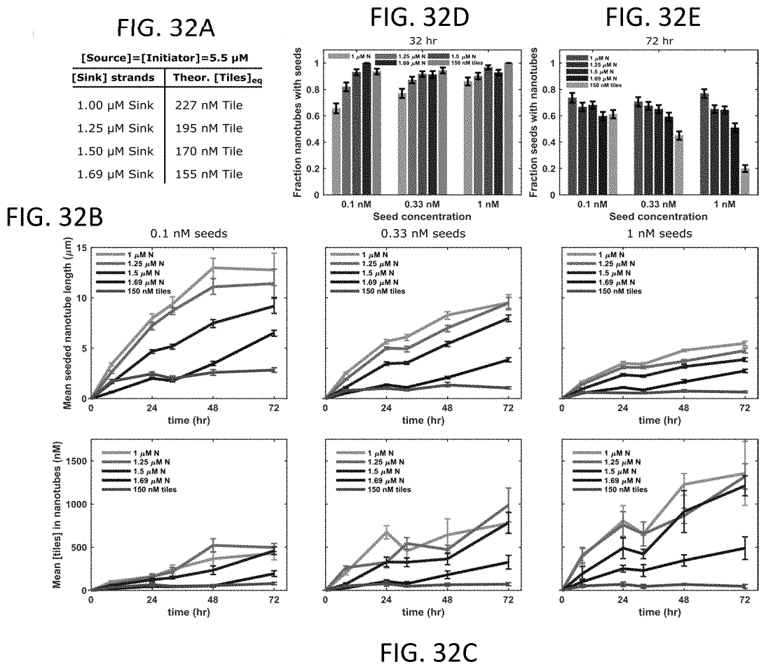

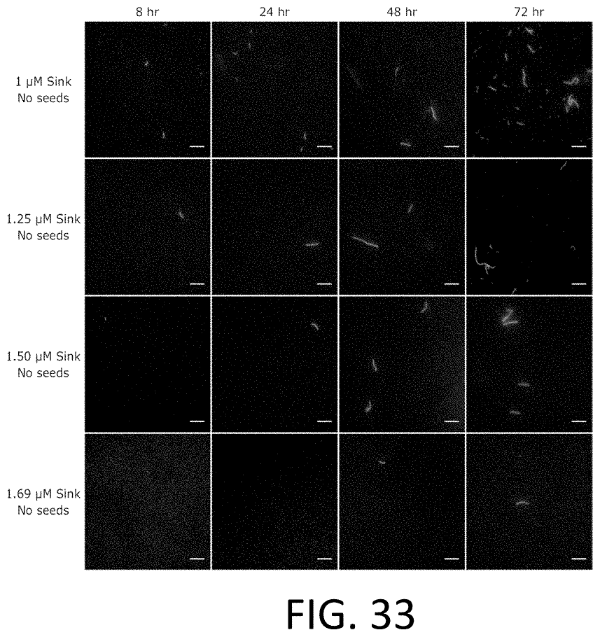

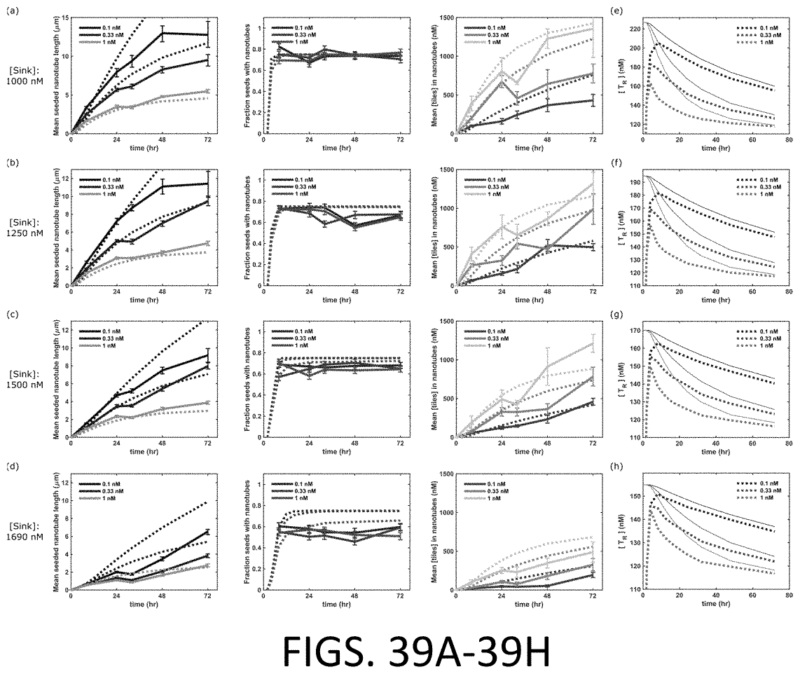

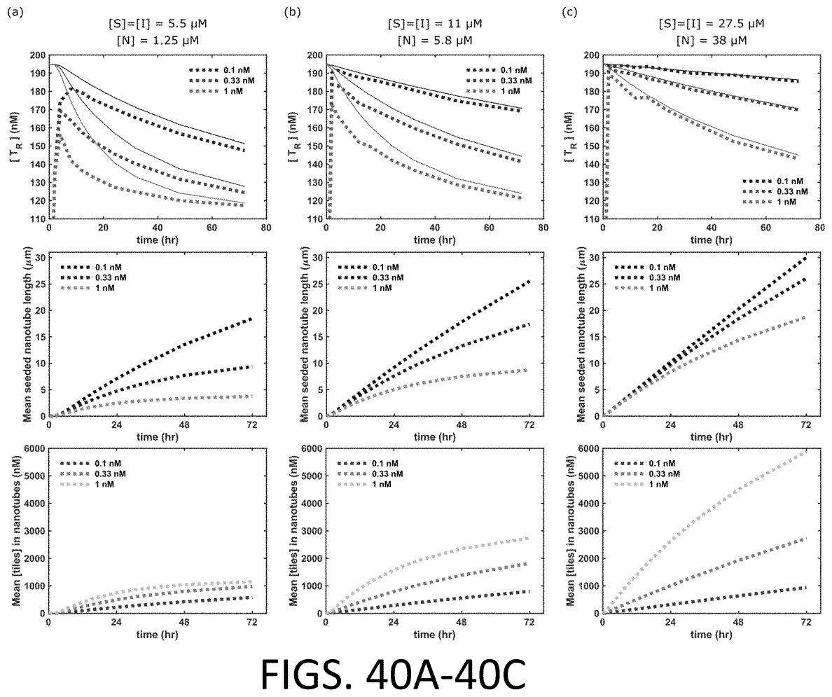

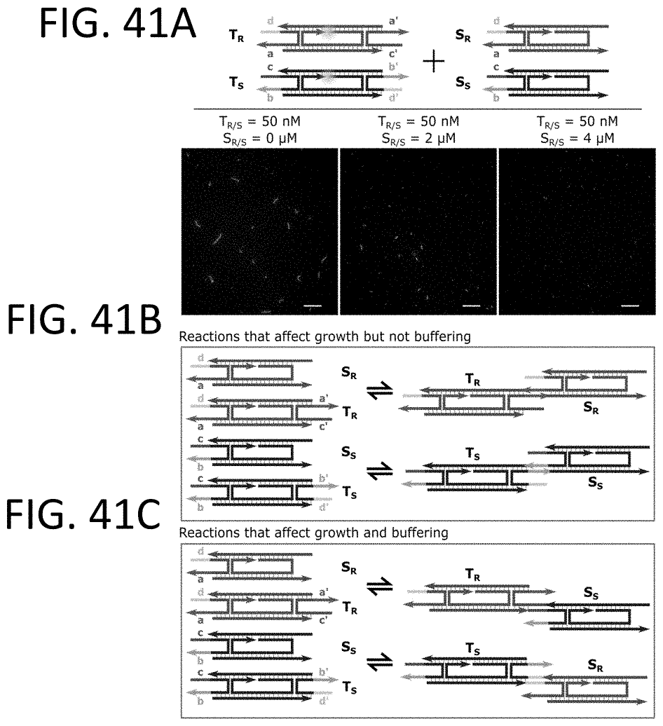

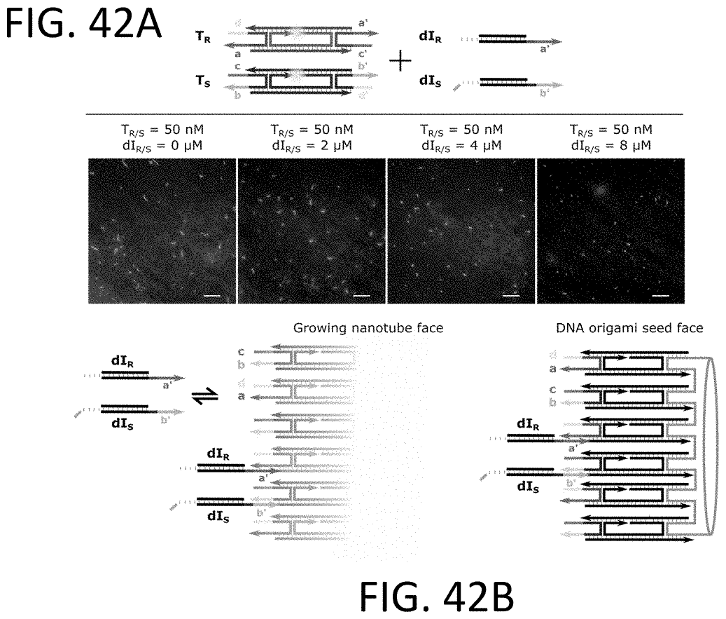

Using the slower tile buffering reaction rates (k_{f}=1×10⁰ M⁻¹s⁻¹ and kᵣ=1×10² M⁻¹s⁻¹), nanotube growth was also simulated with buffering for the different Sink strand concentrations used in the experiments in FIG. 32. Agreement was established with the experimental results for all but the highest Sink strand concentration where the simulations overpredicted the observed growth (FIG. 39).

### Example 21

Buffer Reactions of Orders 0 Through 2:

In principle, a reversible reaction of any order can act as a buffer, provided (1) that one of the products is the molecule X, whose concentration is to be regulated or buffered, (2) all of the other species besides X are present at high concentration relative to the equilibrium concentration of X, (3) there is sufficient control over the forward and reverse rate constants (e.g., for a pH buffer a weak acid with a low dissociation constant was used) to tune the buffer. It was decided to implement a buffer using two reactants and two products because this reaction form provides the ability to finely tune both the forward and reverse reaction rates by adjusting the appropriate reactant concentrations, and because the buffering reaction occurs when the reactants are mixed, making it straightforward to characterize buffering kinetics. Below is a table describing the generalized buffer reactions up to the second order, with a brief summary of the benefits of each form.

TABLE 7
Buffer reaction orders:

| Reaction Order | | | | | |
|---|---|---|---|---|---|
| Left | Right | Buffering | Equilibrium | Concentration | |
| side | side | Reaction | [X]_{eq} | Requirements | Notes |
| 0^{th} | 1^{st} | ${⇌}_{{k}_{r}}^{{k}_{f}} X$ | $\frac{{k}_{f}}{{k}_{r}}$ | — | $\begin{matrix}Infinite capacity . \\ Equivalent to a \\ proportional controller \\ ( gain = \frac{1}{{k}_{r}} , reference = \frac{{k}_{f}}{{k}_{r}} ) .\end{matrix}$ |
| 1^{st} | 1^{st} | $A {⇌}_{{k}_{r}}^{{k}_{f}} X$ | $\frac{{k}_{f} [ A ]}{{k}_{r}}$ | [A]₀ >> [X]_{eq} | Allows the forward rate to be tuned by changing [A]₀ |
| 2^{nd} | 1^{st} | $A + B {⇌}_{{k}_{r}}^{{k}_{f}} X$ | $\frac{{k}_{f} [ A ] [ B ]}{{k}_{r}}$ | [A]₀, [B]₀ >> [X]_{eq} | Prevents the reaction from occurring until A and B are mixed. |
| 0^{th} | 2^{nd} | ${⇌}_{{k}_{r}}^{{k}_{f}} X + C$ | $\frac{{k}_{f}}{{k}_{r} [ C ]}$ | [C]₀ >> [X]_{eq} | Allows the reverse rate to be tuned by changing [C]₀ |
| 1^{st} | 2^{nd} | $A {⇌}_{{k}_{r}}^{{k}_{f}} X + C$ | $\frac{{k}_{f} [ A ]}{{k}_{r} [ C ]}$ | [A]₀, [C]₀ >> [X]_{eq} | Standard form for an acid-base pH buffer |
| 2^{nd} | 2^{nd} | $A + B {⇌}_{{k}_{r}}^{{k}_{f}} X + C$ | $\frac{{k}_{f} [ A ] [ B ]}{{k}_{r} [ C ]}$ | [A]₀, [B]₀, [C]₀ >> [X]_{eq} | Bimolecular form |

Derivations of Setpoint Concentration, Relaxation Time, and Capacity:

The governing equation for the bimolecular buffer reaction is:

$$S + I {{⇁}^{↼}}_{{k}_{N}}^{ {k}_{S} } N + X \qquad ( Eqn 1 )$$

where:
X is the buffered molecule,
S is the source that contains X in an inactive state,
I is the initiator that releases X from S, and
N is the sink that recaptures X.
The mass action ordinary differential equation that governs the kinetics of this reaction is

$$\frac{d [ X ]}{d t} = {k}_{S} [ S ] [ I ] - {k}_{N} [ N ] [ X ] . \qquad ( Eqn 2 )$$

The reaction in Eqn 1 approaches an equilibrium concentration of

$${[ X ]}_{e q} = \frac{{k}_{S}}{{k}_{N}} \frac{{{[ S ]}_{e q} [ I ]}_{e q}}{{[ N ]}_{e q}} = {K}_{e q} \frac{{{[ S ]}_{e q} [ I ]}_{e q}}{{[ N ]}_{e q}} \qquad ( Eqn 3 )$$

where the equilibrium constant K_{eq} is defined as:

$${K}_{e q} ≡ \frac{{k}_{S}}{{k}_{N}} = \frac{{{[ N ]}_{e q} [ X ]}_{e q}}{{{[ S ]}_{e q} [ I ]}_{e q}} . \qquad ( Eqn 4 )$$

Setpoint Concentration: By stoichiometry, the following conservation equations were used:

[S]_{eq}=[S]₀−([X]_{eq}−[X]₀) (Eqn 5)

[I]_{eq}=[I]₀−([X]_{eq}−[X]₀) (Eqn 6)

[N]_{eq}=[N]₀+([X]_{eq}−[X]₀) (Eqn 7)

For very large initial reactant concentrations ([S]₀, [I]₀, [N]₀>>|[X]_{eq}−[X]₀|). In this case the concentrations of S, I and N can be approximated as roughly constant (i.e. [S]_{eq}≈[S]₀, [I]_{eq}≈[I]₀, [N]_{eq}≈[N]₀). This reduces Eqn. Eqn 3 to

$${[ X ]}_{e q} \approx {K}_{e q} \frac{{{[ S ]}_{0} [ I ]}_{0}}{{[ N ]}_{0}} \qquad ( Eqn 8 )$$

Relaxation Time:

If it is assumed that the initial reactant concentrations are sufficiently high, such that they do not change significantly as the system approaches equilibrium ([S]₀, [I]₀, [N]₀>>[X]_{eq}), then treat [S], [I] and [N] can be treated as approximately constant, which reduces Eqn. Eqn 2 to

$$\frac{d [ X ]}{d t} = {k}_{p} - {k}_{d} [ X ] \qquad ( Eqn 9 )$$

where

kₚ≡Production Rate≡k_{S}[S][I] (Eqn 10)

k_{d}≡Degradation Rate≡k_{N}[N] (Eqn 11)

The solution to Eqn 9 is

$$\begin{matrix}[ X ] ( t ) = {[ X \}}_{set} ( 1 + ɛ {e}^{- \frac{t}{τ}} ) \\ where & ( Eqn 12 ) \\ {[ X \}}_{set} ≡ equilibrium setpoint ≡ \frac{{k}_{p}}{{k}_{d}} & ( Eqn 13 ) \\ ɛ ≡ relative offset from setpoint ≡ \frac{{[ X ]}_{0} - {[ X ]}_{set}}{{[ X ]}_{set}} & ( Eqn 14 ) \\ τ ≡ time constant ≡ \frac{1}{{k}_{d}} & ( Eqn 15 )\end{matrix}$$

Eqn 12 can be used to solve for the time it takes to relax to an arbitrary factor α<1 of the initial offset:

$${( 1 + a ɛ ) [ X ]}_{set} = {[ X ]}_{set} ( 1 + ɛ {e}^{- \frac{t}{τ}} ) \qquad ( Eqn 16 )$$

which simplifies to

$$α = {e}^{- \frac{t}{τ}} \qquad ( Eqn 17 )$$

which can be solved to find

t_{relax,α}−τln(α) (Eqn 18)

Alternatively, t=τ can be set and Eqn 18 can be solved to find that each time constant τ is the amount of time to relax to a factor of

$${α}_{t = τ} = \frac{1}{e} \qquad ( Eqn 19 )$$

Capacity:

To calculate the capacity of the buffer, Eqn 3 is first rendered dimensionless

[X]_{eq}≡[X*]_{eq}[ ]_{c} (Eqn20)

which gives

$${[ {X}^{*} ]}_{e q} = {K}_{e q} \frac{{{[ S ]}_{e q} [ I ]}_{e q}}{{{[ N ]}_{e q} [ ]}_{c}} \qquad ( Eqn 21 )$$

where [ ]_{c} is a concentration scale. The following useful quantities can then be defined, analogous to pH and pKa in an acid base buffer. Note that this operation is not permissible without rendering X dimensionless, as logarithms can only operate on dimensionless numbers.

pX≡−log₁₀([X*]_{eq}) (Eqn 22)

pK_{eq}≡−log₁₀(K_{eq}) (Eqn 23)

Taking the −log₁₀ of both sides of Eqn 21, and rearranging, it is found

$$p X = p {K}_{e q} + {log}_{10} ( \frac{{{[ N ]}_{e q} [ ]}_{c}}{{{[ S ]}_{e q} [ I ]}_{e q}} ) \qquad ( Eqn 24 )$$

which is analogous to the Henderson-Hasselbalch equation for acid-base buffers. The capacity 3 is defined as the concentration of X that must be added to the system to change the pX by a given offset γ. Any γ could be selected to define capacity, depending on the magnitude of offset that is relevant to an experiment. In acid base buffers, γ=±1 pH units is often used to define the buffer capacity, but here generally a smaller range is desired. There are two directions to change pX, and therefore two capacities, specifically:

Positive capacity β⁺≡[X] added to decrease pX_{final} to pXᵢₙᵢₜᵢₐₗ+γ, for γ<0 (Eqn 26)

Negative capacity β⁻≡[X] added to decrease pX_{final} to pXᵢₙᵢₜᵢₐₗ+γ, for γ>0 (Eqn 26)

Note that the only way to change pX is through the second term on the right hand side of Eqn 24, which is defined as:

$${δ}_{e q} ≡ {log}_{10} ( \frac{{{[ ]}_{c} [ N ]}_{e q}}{{{[ S ]}_{e q} [ I ]}_{e q}} ) \qquad ( Eqn 27 )$$

Therefore, the positive and negative capacity definitions from Eqn's 25-26 can be restated as the concentrations of X that must be added or removed to change δ_{f}=δ_{eq}+γ. For clarity, the following subscripts are used:
0: initial state of buffer before reaching equilibration,
eq: at initial equilibrium,
f: post-disturbance equilibrium Due to the high concentrations of S, I and N relative to the equilibrium concentration of X, it is assumed that the concentrations of S, I and N do not change significantly during the initial equilibration, i.e. [S, I, N]₀≈[S, I, N]_{eq}. Furthermore, it will be assumed that if a disturbance of ∝≡[X]_{added}=−[Competitor]_{added} is applied to the buffer, then the concentrations of S, I and N change linearly as follows: [S,I]_{f}=[S,I]₀+∝, and [N]_{f}=[N]₀−∝. With these assumptions:

$${δ}_{f} = {δ}_{e q} + γ = {log}_{10} ( \frac{{[ ]}_{c} ( {[ N ]}_{0} - ∝ )}{( {[ S ]}_{0} + ∝ ) ( {[ I ]}_{0} + ∝ )} ) \qquad ( Eqn 28 )$$

Exponentiating both sides of Eqn 28 with base 10, and rearranging into quadratic form, it is found:

a∝²+b∝+c=0 (Eqn 29)

where:

$$a = 1 \\ b = {[ P ]}_{0} + \frac{{[ ]}_{c}}{1 {0}^{{δ}_{e q} + γ}} \\ {[ P ]}_{0} ≡ {[ S ]}_{0} + {[ I ]}_{0} \\ c = {{[ S ]}_{0} [ I ]}_{0} - \frac{{{[ ]}_{c} [ N ]}_{0}}{1 {0}^{{δ}_{e q} + γ}} \qquad ( Eqn 30 )$$

With α=1, the quadratic formula gives the solution to Eqn. 29 as:

$$∝ = \frac{- b \pm \sqrt{{b}^{2} - 4 c}}{2} \qquad ( Eqn 31 )$$

the values in Eqn. 30 can then be substituted back in to find:

$$\begin{matrix}∝ = \frac{- {[ P ]}_{0} - \frac{{[ ]}_{c}}{1 {0}^{{δ}_{e q} + γ}} \pm \sqrt{\begin{matrix} \\ \begin{matrix}\begin{matrix}{[ P ]}_{0}^{2} + {2 [ P ]}_{0} \frac{{[ ]}_{c}}{1 {0}^{{δ}_{e q} + γ}} + \\ \begin{matrix}\frac{{[ ]}_{c}^{2}}{{( {10}^{{δ}_{e q} + γ} )}^{2}} - \\ {{4 [ S ]}_{0} [ I ]}_{0} +\end{matrix}\end{matrix} \\ 4 \frac{{{[ ]}_{c} [ N ]}_{0}}{1 {0}^{{δ}_{e q} + γ}}\end{matrix}\end{matrix}}}{2} \\ notice \\ {10}^{{δ}_{e q} + γ} = 1 {0}^{γ} \cdot \frac{{{[ ]}_{c} [ N ]}_{0}}{{{[ S ]}_{0} [ I ]}_{0}} therefore & ( Eqn 32 ) \\ {( {10}^{{δ}_{e q} + γ} )}^{2} = {10}^{2 γ} \cdot \frac{{{[ ]}_{c}^{2} [ N ]}_{0}^{2}}{{{[ S ]}_{0}^{2} [ I ]}_{0}^{2}} & ( Eqn 33 )\end{matrix}$$

which can then be substituted back in to find:

$$∝ = - \frac{{[ P ]}_{0}}{2} - \frac{{{[ S ]}_{0} [ I ]}_{0}}{2 \cdot {10}^{γ} \cdot {[ N ]}_{0}} \pm \sqrt{\begin{matrix}\frac{{[ P ]}_{0}^{2}}{4} + ( \frac{{[ P ]}_{0} + {2 [ N ]}_{0}}{2 \cdot {10}^{γ} \cdot {[ N ]}_{0}} - 1 ) \\ {{[ S ]}_{0} [ I ]}_{0} + \frac{{{[ S ]}_{0}^{2} [ I ]}_{0}^{2}}{4 \cdot {10}^{2 γ} \cdot {[ N ]}_{0}^{2}}\end{matrix}}$$

Applying the following definitions:

$${[ Total ]}_{0} = {[ S ]}_{0} + {[ I ]}_{0} + {[ N ]}_{0}$$

$${ϕ}_{P} ≡ \frac{{[ P ]}_{0}}{{[ P ]}_{0} + {[ N ]}_{0}} = \frac{{[ P ]}_{0}}{{[ Total ]}_{0}}$$

$${ϕ}_{S} ≡ {\frac{{[ S ]}_{0}}{{[ S ]}_{0} + {[ I ]}_{0}} \\ [ P ]}_{0} = {{{ϕ}_{P} [ Total ]}_{0} \\ [ N ]}_{0} = {{( 1 - {ϕ}_{P} ) [ Total ]}_{0} \\ [ S ]}_{0} = {{ϕ}_{S} [ P ]}_{0} = {ϕ}_{S} {{{ϕ}_{P} [ Total ]}_{0} \\ [ I ]}_{0} = {( 1 - {ϕ}_{S} ) [ P ]}_{0} = ( 1 - {ϕ}_{S} ) {{ϕ}_{P} [ Total ]}_{0}$$

it is found:

$$\frac{∝}{{{ϕ}_{P} [ Total ]}_{0}} = - \frac{1}{2} - \frac{{ϕ}_{S} {ϕ}_{P} - {ϕ}_{s}^{2} {ϕ}_{P}}{2 \cdot {10}^{γ} \cdot ( 1 - {ϕ}_{P} )} \pm \sqrt{\begin{matrix}\frac{1}{4} + ( \frac{{ϕ}_{P} + 2 ( 1 - {ϕ}_{P} )}{2 \cdot {10}^{γ} \cdot ( 1 - {ϕ}_{P} )} - 1 ) ( {ϕ}_{S} - {ϕ}_{S}^{2} ) + \\ \frac{{ϕ}_{S}^{2} {{ϕ}_{P}^{2} ( 1 - {ϕ}_{S} )}^{2}}{4 \cdot {10}^{2 γ} \cdot {( 1 - {ϕ}_{P} )}^{2}}\end{matrix}}$$

which is a general form of the capacity equation, normalized by the initial concentration of S+N. The case where ϕ_{S}=0.5, i.e. [S]₀=[I]₀ was primarily explored, which can be simplified to:

$$\frac{2 ∝}{{{ϕ}_{P} [ Total ]}_{0}} = - 1 - \frac{0.5 {ϕ}_{P}}{2 \cdot {10}^{γ} \cdot ( 1 - {ϕ}_{P} )} \pm \sqrt{\begin{matrix}\begin{matrix}\frac{1}{{10}^{γ} \cdot ( 1 - {ϕ}_{P} )} - \\ \frac{{ϕ}_{P}}{2 \cdot {10}^{γ} \cdot ( 1 - {ϕ}_{P} )} +\end{matrix} \\ \frac{{ϕ}_{P}^{2}}{16 \cdot {10}^{2 γ} \cdot {( 1 - {ϕ}_{P} )}^{2}}\end{matrix}}$$

If select γ±1 was selected, i.e. the capacity to change the equilibrium by a factor of ten from the setpoint concentration, this gives a negative capacity (γ=+1,β⁻≡−∝)) of

$${β}^{-} = \frac{{ϕ}_{P}}{2} {( 1 + \frac{{ϕ}_{P}}{40 \cdot ( 1 - {ϕ}_{P} )} \pm \sqrt{\begin{matrix}\begin{matrix}\frac{1}{10 \cdot ( 1 - {ϕ}_{P} )} - \\ \frac{{ϕ}_{P}}{20 \cdot ( 1 - {ϕ}_{P} )} +\end{matrix} \\ \frac{{ϕ}_{P}^{2}}{1600 \cdot {( 1 - {ϕ}_{P} )}^{2}}\end{matrix}} ) [ Total ]}_{0}$$

and a positive capacity (γ=−1,β⁺≡∝) of:

$${β}^{+} = \frac{{ϕ}_{P}}{2} {( - 1 - \frac{{ϕ}_{P}}{0.4 \cdot ( 1 - {ϕ}_{P} )} \pm \sqrt{\begin{matrix}\frac{10}{( 1 - {ϕ}_{P} )} - \\ \frac{5 {ϕ}_{P}}{( 1 - {ϕ}_{P} )} + \frac{6.25 {ϕ}_{P}^{2}}{{( 1 - {ϕ}_{P} )}^{2}}\end{matrix}} ) [ Total ]}_{0}$$

If interested in the capacity for smaller changes to the equilibrium concentration, a different γ value can be selected. For instance for the negative capacity to shift the equilbrium by a factor of 0.1 in either direction (i.e. [X]eq=0.9[X]ₛₑₜ, and ⋅ [X]_{eq}=1.1[X]ₛₑₜ) a negative capacity is found

$$( γ = {log}_{10} ( \frac{1}{0.9} ) ,$$

$$\frac{- 2 {β}^{-}}{{{ϕ}_{P} [ Total ]}_{0}} = - 1 - \frac{0.5 {ϕ}_{P}}{2 \cdot ( 1 \text{/} 0.9 ) \cdot ( 1 - {ϕ}_{P} )} \pm \sqrt{\begin{matrix}\begin{matrix}\frac{1}{( \frac{1}{0.9} ) \cdot ( 1 - {ϕ}_{P} )} - \\ \frac{{ϕ}_{P}}{2 \cdot ( \frac{1}{0.9} ) \cdot ( 1 - {ϕ}_{P} )} +\end{matrix} \\ \frac{{ϕ}_{P}^{2}}{16 \cdot {10}^{2 log ( \frac{1}{0.9} )} \cdot {( 1 - {ϕ}_{P} )}^{2}}\end{matrix}}$$

and a positive capacity

$$( γ = {log}_{10} ( \frac{1}{1.1} ) ,$$

β⁺≡∝) of:

$$\frac{2 {β}^{+}}{{{ϕ}_{P} [ Total ]}_{0}} = - 1 - \frac{0.5 {ϕ}_{P}}{2 \cdot ( 1 \text{/} 1.1 ) \cdot ( 1 - {ϕ}_{P} )} \pm \sqrt{\begin{matrix}\begin{matrix}\frac{1}{( \frac{1}{1.1} ) \cdot ( 1 - {ϕ}_{P} )} - \\ \frac{{ϕ}_{P}}{2 \cdot ( \frac{1}{1.1} ) \cdot ( 1 - {ϕ}_{P} )} +\end{matrix} \\ \frac{{ϕ}_{P}^{2}}{16 \cdot {10}^{2 log ( \frac{1}{1.1} )} \cdot {( 1 - {ϕ}_{P} )}^{2}}\end{matrix}}$$

These capacities can be rewritten as:

β⁻=c⁻·[Total]₀

β⁺=c⁺·[Total]₀

where for large 100% offsets (γ=±1):

$${c}^{-} ≡ \frac{{ϕ}_{P}}{2} ( 1 + \frac{{ϕ}_{P}}{40 \cdot ( 1 - {ϕ}_{P} )} \pm \sqrt{\begin{matrix}\frac{1}{10 \cdot ( 1 - {ϕ}_{P} )} - \\ \frac{{ϕ}_{P}}{20 \cdot ( 1 - {ϕ}_{P} )} + \frac{{ϕ}_{P}^{2}}{1600 \cdot {( 1 - {ϕ}_{P} )}^{2}}\end{matrix}} )$$

$${c}^{+} ≡ \frac{{ϕ}_{P}}{2} ( - 1 - \frac{{ϕ}_{P}}{0.4 \cdot ( 1 - {ϕ}_{P} )} \pm \sqrt{\begin{matrix}\frac{10}{( 1 - {ϕ}_{P} )} - \\ \frac{5 {ϕ}_{P}}{( 1 - {ϕ}_{P} )} + \frac{6.25 {ϕ}_{P}^{2}}{{( 1 - {ϕ}_{P} )}^{2}}\end{matrix}} )$$

The ±term in the expression for c⁺ must be +, to ensure that β⁺>0. Similarly, the ±term in the expression for c⁻ must be −, to ensure that β⁻<min([S]₀, [I]₀). For smaller 10% offsets

$$( γ = {log}_{10} ( \frac{1}{0.9} ) , or {log}_{10} ( \frac{1}{1.1} ) )$$

$${c}^{-} ≡ \frac{{ϕ}_{P}}{2} ( 1 + \frac{0.5 {ϕ}_{P}}{2 \cdot ( \frac{1}{0.9} ) \cdot ( 1 - {ϕ}_{P} )} \pm \sqrt{\begin{matrix}\begin{matrix}\frac{1}{( \frac{1}{0.9} ) \cdot ( 1 - {ϕ}_{P} )} - \\ \frac{{ϕ}_{P}}{2 \cdot ( \frac{1}{0.9} ) \cdot ( 1 - {ϕ}_{P} )} +\end{matrix} \\ \frac{{ϕ}_{P}^{2}}{16 \cdot {10}^{2 log ( \frac{1}{0.9} )} \cdot {( 1 - {ϕ}_{P} )}^{2}}\end{matrix}} )$$

$${c}^{+} ≡ \frac{{ϕ}_{P}}{2} ( - 1 - \frac{0.5 {ϕ}_{P}}{2 \cdot ( \frac{1}{1.1} ) \cdot ( 1 - {ϕ}_{P} )} \pm \sqrt{\begin{matrix}\begin{matrix}\frac{1}{( \frac{1}{1.1} ) \cdot ( 1 - {ϕ}_{P} )} - \\ \frac{{ϕ}_{P}}{2 \cdot ( \frac{1}{1.1} ) \cdot ( 1 - {ϕ}_{P} )} +\end{matrix} \\ \frac{{ϕ}_{P}^{2}}{16 \cdot {10}^{2 log ( \frac{1}{1.1} )} \cdot {( 1 - {ϕ}_{P} )}^{2}}\end{matrix}} )$$

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

Sequences referenced herein are provided in an accompanying sequence listing.

What is claimed is:

1. A composition for modulating concentration of a polynucleotide, the composition comprising:
a source complex comprising a single-stranded target polynucleotide; and
a single-stranded initiator polynucleotide capable of associating with the source complex to displace the target polynucleotide from the source complex;
wherein the concentration of the target polynucleotide is modulated by altering the concentrations of at least one of the source complex or the initiator polynucleotide.

2. The composition according to claim 1, further comprising a sink complex, wherein the sink complex comprises a double-stranded polynucleotide comprising the single-stranded initiator polynucleotide and a complementary single-stranded polynucleotide, wherein the complementary single-stranded polynucleotide is at least partially complementary to both the target polynucleotide and the initiator polynucleotide.

3. The composition according to claim 1 or claim 2, wherein the source complex comprises a double-stranded polynucleotide comprising the single-stranded target polynucleotide and a complementary single-stranded polynucleotide, wherein the complementary single-stranded polynucleotide is at least partially complementary to both the target polynucleotide and the initiator polynucleotide.

4. The composition according to any of claims 1 to 3, wherein the initiator polynucleotide is at a concentration ranging from about 100 nM to about 1 mM.

5. The composition according to claim 2, wherein the double-stranded polynucleotide comprising the single-stranded initiator polynucleotide and a complementary single-stranded polynucleotide is at a concentration ranging from about 100 nM to about 1 mM.

6. The composition according to claim 3, wherein the double-stranded polynucleotide comprising the single-stranded target polynucleotide and a complementary single-stranded polynucleotide is at a concentration ranging from about 100 nM to about 1 mM.

7. The composition according to any of claim 1 to 6, wherein the concentration of the initiator polynucleotide, the concentration of the double-stranded polynucleotide comprising the single-stranded initiator polynucleotide and a complementary single-stranded polynucleotide, and the concentration of the double-stranded polynucleotide comprising the single-stranded target polynucleotide and a complementary single-stranded polynucleotide are higher than the concentration of the single-stranded target polynucleotide.

8. The composition according to any of claims 1 to 7, wherein the target polynucleotide comprises from about 10 to about 100 nucleotides.

9. The composition according to any of claims 1 to 8, wherein the initiator polynucleotide comprises from about 10 to about 100 nucleotides.

10. The composition according to any of claims 1 to 9, wherein the target polynucleotide and the initiator polynucleotide comprise at least one toehold domain.

11. The composition according to claims 1 to 9, wherein the toehold domain comprises from about 0 to about 10 nucleotides.

12. The composition according to any of claims 1 to 11, further comprising a reporter complex comprising a reporter molecule.

13. The composition according to claim 12, wherein the reporter complex comprises a double-stranded polynucleotide comprising a single-stranded reporter polynucleotide and a complementary single-stranded quencher polynucleotide, wherein the reporter polynucleotide is at least partially complementary to both the quencher polynucleotide and the target polynucleotide.

14. The composition according to claim 12, wherein the reporter molecule is selected from the group consisting of a bioluminescent agent, a chemiluminescent agent, a chromogenic agent, a fluorogenic agent, an enzymatic agent and combinations or derivatives thereof.

15. The composition according to any of claims 12 to 14, wherein the reporter polynucleotide comprises from about 10 to about 100 nucleotides.

16. The composition according to any of claims 1 to 11, further comprising a competitor complex.

17. The composition according to claim 16, wherein the competitor complex comprises a double-stranded polynucleotide comprising a first single-stranded competitor polynucleotide and a second complementary single-stranded competitor polynucleotide, wherein the first competitor polynucleotide is at least partially complementary to both the second competitor polynucleotide and the target polynucleotide.

18. The composition according to claim 16 or claim 17, wherein the competitor polynucleotide comprises from about 10 to about 100 nucleotides.

19. The composition according to any of claims 1 to 18, wherein the target polynucleotide and the initiator polynucleotide comprise at least one of a DNA molecule, an RNA molecule, a modified nucleic acid, or a combination thereof.

20. A method of modulating concentration of a polynucleotide, the method comprising:
formulating a composition comprising a source complex comprising a single-stranded target polynucleotide and a single-stranded initiator polynucleotide capable of associating with the source complex to displace the target polynucleotide from the source complex; and
increasing or decreasing the concentration of the initiator polynucleotide in the composition to modulate the concentration of the target polynucleotide.

21. The method according to claim 20, further comprising a sink complex, wherein the sink complex comprises a double-stranded polynucleotide comprising the single-stranded initiator polynucleotide and a complementary single-stranded polynucleotide, wherein the complementary single-stranded polynucleotide is at least partially complementary to both the target polynucleotide and the initiator polynucleotide.

22. The method according to claim 20 or claim 21, wherein the source complex comprises a double-stranded polynucleotide comprising the single-stranded target polynucleotide and a complementary single-stranded polynucleotide, wherein the complementary single-stranded polynucleotide is at least partially complementary to both the target polynucleotide and the initiator polynucleotide.

23. The method according to any of claims 20 to 22, further comprising a reporter complex comprising a reporter molecule, wherein the reporter complex comprises a double-stranded polynucleotide comprising a single-stranded reporter polynucleotide and a complementary single-stranded quencher polynucleotide, wherein the reporter polynucleotide is at least partially complementary to both the quencher polynucleotide and the target polynucleotide.

24. The method according to any of claims 20 to 23, further comprising a competitor complex, wherein the competitor complex comprises a double-stranded polynucleotide comprising a first single-stranded competitor polynucleotide and a second complementary single-stranded competitor polynucleotide, wherein the first competitor polynucleotide is at least partially complementary to both the second competitor polynucleotide and the target polynucleotide.

25. The method according to any of claims 20 to 24, wherein the modulation of the target polynucleotide comprises increasing the concentration of the target polynucleotide, wherein the target polynucleotide displaces a small molecule target bound to an aptamer by binding to at least a portion of the aptamer.

26. The method according to any of claims 20 to 25, wherein the modulation of the target polynucleotide comprises increasing the concentration of the target polynucleotide, wherein the target polynucleotide alters one or more conformation properties of a nucleic acid-based hydrogel, or a nucleic acid within a hydrogel or conjugated to a hydrogel or solid support.

27. A system for modulating concentration of two or more polynucleotides, the system comprising:
a first composition comprising a first source complex comprising a first single-stranded target polynucleotide and a first single-stranded initiator polynucleotide capable of associating with the first source complex to displace the first target polynucleotide from the first source complex; and
at least a second composition comprising a second source complex comprising a second single-stranded target polynucleotide and a second single-stranded initiator polynucleotide capable of associating with the second source complex to displace the second target polynucleotide from the second source complex;
wherein the concentrations of the first and second target polynucleotides are modulated independently within the system by altering the concentrations of at least one of the first and second source complexes or the first and second initiator polynucleotides.

28. A composition for modulating nanostructure growth, the composition comprising:
a first source complex comprising two or more single-stranded polynucleotides;
a second source complex comprising two or more single-stranded polynucleotides; and
an initiator complex comprising two or more single-stranded polynucleotides capable of associating with the first and/or second source complex to displace a polynucleotide from the first or the second source complex;
wherein the growth of the nanostructure is modulated by altering a concentration of a polynucleotide of at least one of the first and/or second source complex or the initiator complex.

29. A system for modulating nanostructure growth, the system comprising:
a first composition comprising a first source complex that is a first inactive nanostructure monomer comprising two or more single-stranded polynucleotides, a first sink complex comprising one or more single-stranded polynucleotides, and a first initiator complex comprising two or more single-stranded polynucleotides that is capable of associating with the first source complex to produce a first active nanostructure monomer; and
at least a second composition comprising a second source complex that is a second inactive nanostructure monomer comprising two or more single-stranded polynucleotides, a second sink complex comprising one or more single-stranded polynucleotides, and a second initiator complex comprising two or more single-stranded polynucleotides that is capable of associating with the second source complex to produce a second active nanostructure monomer;
wherein the growth of the nanostructure is modulated by independently altering the concentration the first and second active monomers by changing the concentrations of at least one of the first and second source complexes the first and second initiator complexes or the first and second sink complexes.
